(19) United States
(12) Patent Application Publication
(10) Pub. No.: US 2005/0107381 A1
(43) Pub. Date: May 19, 2005

(54) 6-membered heteroaryl compounds for the treatment of neurodegenerative disorders

(76) Inventor: Yuhpyng L. Chen, Waterford, CT (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/909,474

(22) Filed: Aug. 2, 2004

Related U.S. Application Data
(60) Provisional application No. 60/492,088, filed on Aug. 1, 2003.

(51) Int. Cl. A61K 31/52; A61K 31/501; A61K 31/506; A61K 31/4965

(52) U.S. Cl. 514/241; 514/247; 514/255.06; 514/275; 514/352; 544/209; 544/329; 544/406; 546/309

(57) ABSTRACT

The present invention relates to compounds of the Formula 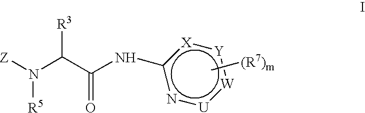
wherein R¹, R^{1a}, R^{1b}, R², R³, R⁴, R⁵, R⁶, R⁷, R⁸, R⁹, R¹⁰, R¹¹, R¹², X, Y, W, U, Z, m and n are as defined. Compounds of the Formula I have activity inhibiting production of Aβ-peptide. This invention also relates to pharmaceutical compositions and methods of treating diseases, for example, neurodegenerative diseases, e.g., Alzheimer's disease, in a mammal comprising compounds of the Formula I.

### CROSS REFERENCE TO RELATED APPLICATION

The present application claims benefit of U.S. Ser. No. 60/492,088 filed on Aug. 1, 2003.

### FIELD OF THE INVENTION

The present invention relates to the treatment of Alzheimer's disease and other neurodegenerative and/or neurological disorders in mammals, including humans. This invention also relates to inhibiting, in mammals, including humans, the production of Aβ-peptides that can contribute to the formation of neurological deposits of amyloid protein. More particularly, this invention relates to 6-membered heteroaryl compounds useful for the treatment of neurodegenerative and/or neurological disorders, such as Alzheimer's disease and Down's Syndrome, related to Aβ-peptide production.

### BACKGROUND OF THE INVENTION

Dementia results from a wide variety of distinctive pathological processes. The most common pathological processes causing dementia are Alzheimer's disease (AD), cerebral amyloid angiopathy (CAA) and prion-mediated diseases (see, e.g., Haan et al. Clin. Neurol. Neurosurg. 1990, 92(4):305-310; Glenner et al. J Neurol. Sci. 1989, 94:1-28). AD affects nearly half of all people past the age of 85, the most rapidly growing portion of the United States population. As such, the number of AD patients in the United States is expected to increase from about 4 million to about 14 million by the middle of the next century.

Treatment of AD typically is the support provided by a family member in attendance. Stimulated memory exercises on a regular basis have been shown to slow, but not stop, memory loss. A few drugs, for example, Aricep™, provide treatment of AD.

A hallmark of AD is the accumulation in the brain of extracellular insoluble deposits called amyloid plaques and abnormal lesions within neuronal cells called neurofibrillary tangles. Increased plaque formation is associated with an increased risk of AD. Indeed, the presence of amyloid plaques, together with neurofibrillary tangles, are the basis for definitive pathological diagnosis of AD.

The major components of amyloid plaques are the amyloid Aβ-peptides, also called Aβ-peptides, which consist of three proteins having 40, 42 or 43 amino acids, designated as the Aβ₁₋₄₀, Aβ₁₋₄₂, and Aβ₁₋₄₃ peptides, respectively. The Aβ-peptides are thought to cause nerve cell destruction, in part, because they are toxic to neurons in vitro and in vivo.

The Aβ peptides are derived from larger amyloid precursor proteins (APP proteins), which consist of four proteins containing 695, 714, 751 or 771 amino acids, designated as the APP₆₉₅, APP₇₁₄, APP₇₅₁, and APP₇₇₁, respectively. Proteases are believed to produce the Aβ peptides by cleaving specific amino acid sequences within the various APP proteins. The proteases are named “secretases” because the Aβ-peptides they produce are secreted by cells into the extracellular environment. These secretases are each named according to the cleavage(s) they make to produce the Aβ-peptides. The secretase that forms the amino terminal end of the Aβ-peptides is called the beta-secretase. The secretase that forms the carboxyl terminal end of the Aβ-peptides is called the gamma-secretase (Haass, C. and Selkoe, D. J. 1993 Cell 75:1039-1042).

This invention relates to novel compounds that inhibit Aβ-peptide production, to pharmaceutical compositions comprising such compounds, and to methods of using such compounds to treat neurodegenerative and/or neurological disorders.

### SUMMARY OF THE INVENTION

The present invention relates to compounds of the formula 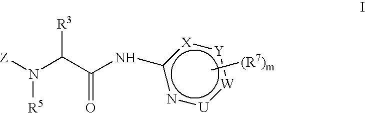
wherein Z is selected from —C(═O)CHR¹R², —C(═S)CHR¹R², —(C═NR⁸)CHR¹R², —C(═O)C(═O)R¹, —SO₂—R¹ and R¹;
m is an integer independently selected from zero, 1, 2, and 3;
R¹ is selected from —C₁-C₂₀ alkyl, —C₂-C₂₀ alkenyl, —C₂-C₂₀ alkynyl, —C₁-C₂₀ alkoxy, —C₂-C₂₀ alkenoxy, —C₂-C₂₀ alkynoxy, —C₃-C₂₀ cycloalkyl, —C₄-C₂₀ cycloalkenyl, —(C₁₀-C₂₀)bi- or tricycloalkyl, —(C₁₀-C₂₀)bi- or tricycloalkenyl, -(4-20 membered)heterocycloalkyl, —C₆-C₂₀ aryl and -(5-20 membered)heteroaryl;
wherein R¹ is optionally independently substituted with from one to six fluorine atoms or with from one to three substituents independently selected from the group R^{1a};
R^{1a} is in each instance independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —Cl, —Br, —I, —CN, —NO₂, —NR⁹R¹⁰, —C(═O)NR⁹R¹⁰, —SO₂—NR⁹R¹⁰, —C(═O)R¹¹, —SO₂—R¹¹, —C(═O)OR¹², —C₃-C₁₅ cycloalkyl, —C₄-C₁₅ cycloalkenyl, —(C₅-C₁₁)bi- or tricycloalkyl, —(C₇-C₁₁)bi- or tricycloalkenyl, -(4-20 membered) heterocycloalkyl, —C₆-C₁₅ aryl, -(5-15 membered)heteroaryl, —C₆-C₁₅ aryloxy and -(5-15 membered)heteroaryloxy, wherein said cycloalkyl, cycloalkenyl, bi- or tricycloalkyl, bi- or tricycloalkenyl, heterocycloalkyl, aryl, heteroaryl, aryloxy and heteroaryloxy of R^{1a} are each optionally independently substituted with from one to three substituents independently selected from the group R^{1b};
R^{1b} is in each instance independently selected from —OH, —C₁-C₆ alkyl, —C₂-C₆ alkenyl, —C₂-C₆ alkynyl, —C₁-C₆ alkoxy, —C₂-C₆ alkenoxy, —C₂-C₆ alkynoxy, —C₁-C₆ hydroxyalkyl, —F, —Cl, —Br, —I, —CN, —NO₂, —NR⁹R¹⁰, —C(═O)NR⁹R¹⁰, —C(═O)R¹¹, —SO₂—R¹¹, —C₆-C₁₅ aryloxy and -(5-15 membered)heteroaryloxy, wherein said alkyl, alkenyl and alkynyl of R^{1b} are each optionally independently substituted with from one to six fluorine atoms or with from one to two substituents independently selected from —C₁-C₄ alkoxy, or with a hydroxy group;
R⁹ and R¹⁰ are in each instance each independently selected from —H, —C₁-C₁₂ alkyl, —C₂-C₁₂ alkenyl, —C₂-C₁₂ alkynyl, CF₃, —C(═O)R¹¹, —SO₂—R¹¹, —C(═O)OR¹², —C(═O)NR¹¹R¹², —SO₂—NR¹¹R², —(C_{zero}-C₄ alkylene)-(C₃-C₂₀ cycloalkyl), —(C_{zero}-C₄ alkylene)-(C₄-C₈ cycloalkenyl), —(C_{zero}-C₄ alkylene)-((5-10 membered)heterocycloalkyl), (C_{zero}-C₄ alkylene)-(C₆-C₁₀ aryl) and —(C_{zero}-C₄ alkylene)-((5-10 membered)heteroaryl), wherein said alkyl, alkenyl and alkynyl of R⁹ and R¹⁰ are each optionally independently substituted with from one to six fluorine atoms or with from one to two substituents independently selected from —C₁-C₄ alkoxy, with a hydroxy group, or C(═O)OR¹², and wherein said cycloalkyl, cycloalkenyl, bi- or tricycloalkyl, bi- or tricycloalkenyl, heterocycloalkyl, aryl and heteroaryl are each optionally independently substituted with from one to three substituents independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —C₁-C₆ hydroxyalkyl, —F, —Cl, —Br, —I, —CN, —NO₂, —CF₃, —NH₂, —C(═O)NH₂, —SO₂—NH₂, —C(═O)H and —C(═O)OH, wherein said alkyl, alkenyl and alkynyl substituents are each optionally independently further substituted with from one to six fluorine atoms or with from one to two substituents independently selected from —C₁-C₄ alkoxy, or with a hydroxy group or aryl group;
or NR⁹R¹⁰ may in each instance independently optionally form a heterocycloalkyl moiety of from four to ten ring members, said heterocycloalkyl moiety optionally containing one to two further heteroatoms independently selected from N, O and S, and optionally containing from one to three double bonds, wherein the carbon atoms of the heterocycloalkyl moiety of NR⁹R¹⁰ are optionally independently substituted with from one to three substituents independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —F, —Cl, —Br, —I, —CF₃, —NH₂, —C(═O)NH₂, —SO₂—NH₂, —C(═O)R¹¹, SO₂—R¹¹, (C_{zero}-C₄ alkylene)-(C₆-C₁₀ aryl), (C_{zero}-C₄ alkylene)-((5-10 membered heteroaryl), (C_{zero}-C₄ alkylene)-(C₆-C₁₀ cycloalkyl) and (C_{zero}-C₄ alkylene)-((5-10 membered)heterocycloalkyl), and wherein the (C_{zero}-C₄ alkylene)-((5-10 membered)heterocycloalkyl) substituent and the nitrogen atoms of said heterocycloalkyl moiety of NR⁹R¹⁰ are each optionally independently substituted with one substituent independently selected from —C₁-C₁₂ alkyl, —C₂-C₁₂ alkenyl, —C₂-C₁₂ alkynyl, —C(═O)NH₂, —SO₂—NH₂, C(═O)R¹¹, SO₂—R¹¹, (C_{zero}-C₄ alkylene)-(C₆-C₁₀ aryl), (C_{zero}-C₄ alkylene)-((5-10 membered)heteroaryl), (C_{zero}-C₄ alkylene)-(C₆-C₁₀ cycloalkyl) and (C_{zero}-C₄ alkylene)-((5-10 membered)heterocycloalkyl), and wherein said alkyl, alkenyl and alkynyl substituents are each optionally independently further substituted with from one to six fluorine atoms, or with from one to two substituents independently selected from —C₁-C₄ alkoxy, or with a hydroxy group;
R¹¹ and R¹² are in each instance each independently selected from hydrogen, —C_{zero}-C₁₋₅ alkyl, —C₂-C₆ alkenyl, —C₂-C₆ alkynyl, —(C_{zero}-C₄ alkylene)-(C₃-C₁₅ cycloalkyl), —(C_{zero}-C₄ alkylene)-(C₄-C₈ cycloalkenyl), —(C_{zero}-C₄ alkylene)-(C₆-C₁₅ aryl), —(C_{zero}-C₄ alkylene)-((5-15 membered) heterocycloalkyl) and C_{zero}-C₄ alkylene)-((5-15 membered)heteroaryl);
wherein R¹¹ and R¹² are each optionally independently substituted with from one to three substituents independently selected from the group R^{1b};
R² is selected from —H, —OH, —NH₂, —CH₂OH, —OC(═O)CH₃, —C(CH₃)₂OH, —C(CH₃)(CH₂CH₃)(OH), —C(OH)(C_{zero}-C₄ alkyl)(C_{zero}-C₄ alkyl), —OC(═O)R⁴ and —OC(═O)OR⁴, wherein said —OC(═O)R⁴ and —OC(═O)OR⁴ may optionally be a prodrug of the corresponding OH of R²;
R⁴ is selected from —C₁-C₄ alkyl, —CH(OH)(C₁-C₄ alkyl), —CH(OH)(C₅-C₆ aryl), —CH(OH)((5-6 membered)heteroaryl), —CH(OH)(C₅-C₆ cycloalkyl) and —CH(OH)((5-6 membered) heterocycloalkyl);
R³ is selected from —C₁-C₆ alkyl, —C₂-C₆ alkenyl, —C₂-C₆ alkynyl and C_{zero}-C₄ alkylene)-(C₃-C₆ cycloalkyl), wherein when R³ is alkyl, alkenyl or alkynyl, R³ is optionally independently substituted with a substituent independently selected from —C₁-C₄ alkoxy, —F, —OH and —S(C₁-C₄ alkyl);
R⁵ is selected from —H, —C₁-C₄ alkyl, —C₂-C₄ alkenyl, —C₂-C₄ alkynyl, —C(═O)(C₁-C₄ alkyl), —C₆-C₁₀ aryl, -((5-20 membered)heteroaryl), —SO₂—(C₆-C₁₀ aryl), —SO₂-((5-20 membered) heteroaryl), —SO₂—CH₂—(C₆-C₂₀ aryl) and —SO₂—CH₂-((5-20 membered)heteroaryl);
R⁷ is selected from —H, —C₁-C₂₀ alkyl, —C₂-C₂₀ alkenyl, —C₂-C₂₀ alkynyl, —C₁-C₂₀ alkoxy, —C₂-C₂₀ alkenoxy, —C₂-C₂₀ alkynoxy, —F, —Cl, —Br, —I, —CN, —NO₂, —OH, —CF₃, —NR⁹R¹⁰, (C₆-C₁₈)aryloxy, —(C₁-C₁₁ alkylene)-NR⁹R¹⁰, —C(═O)NR⁹R¹⁰, —C(═O)R¹¹, —CHO, —SO₂—R¹¹, —C(═O)OR¹², COOH, C_{zero}-C₄ alkylene)-(C₃-C₂₀ cycloalkyl), C_{zero}-C₄ alkylene)-(C₄-C₂₀ cycloalkenyl), —(C_{zero}-C₄ alkylene)-((C₁₀-C₂₀)bi- or tricycloalkyl), —(C_{zero}-C₄ alkylene)-((C₁₀-C₂₀)bi- or tricycloalkenyl), —(C_{zero}-C₄ alkylene)-((3-20 membered)heterocycloalkyl), C_{zero}-C₄ alkylene)-(C₆-C₁₅ aryl) and —(C_{zero}-C₄ alkylene)-((5-15 membered)heteroaryl), wherein said heterocycloalkyl optionally contains from one to four ring double or triple bonds;
wherein R⁷ is optionally substituted with from one to six fluorine atoms or with from one to three substituents independently selected from the group R^{1a};
or two independently selected R⁷ groups may, together with the carbon atoms to which they are respectively attached, optionally form a five to fourteen membered cycloalkyl ring, a five to fourteen membered heterocycloalkyl ring, wherein from one to three members of said heterocycloalkyl ring are selected from N, O and S, and wherein said cycloalkyl, heterocycloalkyl, bicycloalkyl or bicylcoheteroalkyl ring optionally contains from one to three double bonds;
X, Y, W and U are each independently selected from carbon and nitrogen, wherein the heteroaryl ring of Formula I that contains X, Y, W and U or the heteroaryl ring containing two R⁷ groups taken together with the carbon atoms on the heteroaryl ring of the Formula containing X, Y, W and U may be, but is not limited to, pyridyl, pyrimidyl, pyrazinyl, pyridazinyl, quinolinyl, quinazolinyl, quinoxalinyl, cyclopenta-pyrimidine and dihydropyrrolo-pyrimidine with the proviso that the 6-membered heteroaryl ring of Formula I that contains X, Y, W and U many not contain more than three nitrogen atoms in the ring, and with the further proviso that no more than two nitrogen atoms in the 6-membered heteroaryl ring of Formula I that contains X, Y, W and U may be directly adjacent to each other in the ring, and with the further proviso when R⁷ is —OH and R⁷ is attached to a carbon atom of the 6-membered heteroaryl ring Formula I that contains X, Y, W and U, and m is 1, 2 or 3, then the —OH group of R⁷ that is attached to a carbon atom of the 6-membered heteroaryl ring of Formula I may be tautomerizable to a C═O group;
R⁸ is selected from —H and —C₁-C₆ alkyl;
or, when Z is —C(═NR⁸)CHR¹R², R⁸ and R¹ may together with the nitrogen and carbon atoms to which they are respectively attached optionally form a five to fourteen membered heteroaryl ring or a five to eight membered heterocycloalkyl ring, wherein said heteroaryl or heterocycloalkyl ring optionally contains from one to two further heteroatoms selected from N, O and S, and wherein said heterocycloalkyl ring optionally contains from one to three double bonds, and wherein said heteroaryl or heterocycloalkyl ring is optionally substituted with from one to three substituents independently selected from the group R^{1b};
and the pharmaceutically acceptable salts of such compounds.

Compounds of the Formula I may have optical centers and therefore may occur in different enantiomeric, diastereomeric, meso configurations and in geometric isomers Z or E. The present invention includes all enantiomers, diastereomers, and other stereoisomers of such compounds of the Formula I, as well as racemic and other mixtures thereof.

However, a preferred embodiment of Formula I has the formula 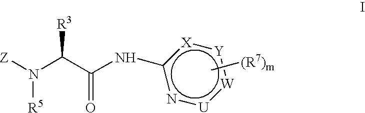
wherein Z, R⁵, R³, X, Y, W, U, R⁷ and m are as defined hereinabove.

Insofar as the compounds of Formula I of this invention contain basic groups, they can form acid addition salts with various inorganic and organic acids. The present invention also relates to the pharmaceutically acceptable acid addition salts of compounds of the Formula I. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent, and thereafter, convert the free base to a pharmaceutically acceptable acid addition salt. The acid addition salts of the base compounds of this invention are readily prepared by treating the base compound with a substantially equivalent amount of the chosen mineral or organic acid in an appropriate solvent such as chloroform, methylene chloride, isopropyl ether, diethyl ether, tetrahydrofuran, toluene, acetonitrile, dioxane, methanol, isopropanol, ethyl acetate, propanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as chloride, bromide, iodide, trifluoroacetate, formic acid, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts. Other examples of pharmaceutically acceptable salts of the compounds of this invention are the salts of salicylic acid, oxalic acid, di-p-toluoyl tartaric acid, mandelic acid, sodium, potassium, magnesium, calcium and lithium.

The present invention also includes isotopically-labeled compounds that are identical to those recited in Formula I, but for the fact that one or more atoms are replaced by an atom having an atomic mass or mass number different from the atomic mass or mass number usually found in nature. Examples of isotopes that can be incorporated into the compounds of the present invention include isotopes of hydrogen, carbon, nitrogen, oxygen, phosphorous, fluorine, chlorine and iodine, such as ²H, ³H, ¹³C, ¹¹C, ¹⁴C, ¹⁵N, ¹⁸O, ¹⁷O, ¹⁸F, ¹²³1 and ¹²⁵I, respectively. The compounds of Formula I of the present invention, prodrugs thereof, pharmaceutically acceptable salts of such compounds or of such prodrugs, and compounds and derivatives of such compounds that contain the aforementioned isotopes and/or other isotopes are within the scope of this invention. Such compounds may be useful as research and diagnostic tools in metabolism pharmacokinetic studies and in binding assays. Certain isotopically-labeled compounds of the Formula I of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically-labeled compounds of the Formula I of the present invention and prodrugs and derivatives thereof may generally be prepared by carrying out the procedures disclosed in the schemes and discussion of the schemes and/or in the examples and preparations described herein, by substituting a readily available isotopically-labeled reagent for a nonisotopically-labeled reagent in the preparation of said compounds.

Unless otherwise indicated, as used herein, the terms “halogen” and “halo” include F, Cl, Br, and 1.

Unless otherwise indicated, as used herein, the term “alkyl” includes saturated monovalent hydrocarbon radicals having straight or branched moieties. Examples of alkyl groups include, but are not limited to, methyl, ethyl, n-propyl, isopropyl, cyclopropylmethylene (—CH₂-cyclopropyl) and t-butyl, and the like.

Unless otherwise indicated, as used herein, the term “alkenyl” includes alkyl moieties having at least one carbon-carbon double bond wherein alkyl is as defined above. Examples of alkenyl include, but are not limited to, ethenyl and propenyl, and the like.

Unless otherwise indicated, as used herein, the term “alkynyl” includes alkyl moieties having at least one carbon-carbon triple bond wherein alkyl is as defined above. Examples of alkynyl groups include, but are not limited to, ethynyl and 2-propynyl, and the like.

Unless otherwise indicated, as used herein, the term “alkoxy”, means “alkyl-O-”, wherein “alkyl” is as defined above. Examples of “alkoxy” groups include, but are not limited to, methoxy, ethoxy, propoxy, butoxy, pentoxy and allyloxy.

Unless otherwise indicated, as used herein, the term “alkenoxy”, means “alkenyl-O-”, wherein “alkenyl” is as defined above.

Unless otherwise indicated, as used herein, the term “alkynoxy”, means “alkynyl-O-”, wherein “alkynyl” is as defined above.

In all of the above defined “C₁-Cₓ alkyl,” “C₁-Cₓ alkenyl,” “C₁-Cₓ alkynyl,” “C₁-Cₓ alkoxy,” “C₁-Cₓ alkenoxy,” and “C₁-Cₓ alkynoxy,” groups, when x is an integer greater than 2, such “C₁-Cₓ alkyl,” “C₁-Cₓ alkenyl,” “C₁-Cₓ alkynyl,” “C₁-Cₓ alkoxy,” “C₁-Cₓ alkenoxy,” and “C₁-Cₓ alkynoxy,” groups, may optionally be replaced with a “polyfluoro C₁-Cₓ alkyl,” a polyfluoro C₁-Cₓ alkenyl,” a “polyfluoro C₁-Cₓ alkynyl,” a “polyfluoro C₁-Cₓ alkoxy,” a “polyfluoro C₁-Cₓ alkenoxy,” or a “polyfluoro C₁-Cₓ alkynoxy,” group. As used herein, the expression “polyfluoro C₁-Cₓ alkyl refers to alkyl groups, as defined above, that comprise at least one —CF₂ and/or CF₃ group.

As defined herein C₀-Cₓ alkylene refers to a covalent bond (C₀) or alkylene bridge (Cₓ), wherein “alkylene” refers to a bridging hydrocarbyl group, that is a group containing carbon and hydrogen, which may be optionally substituted as described herein. It contains 1 less hydrogen atom than the corresponding alkyl group. It may be straight chain or branched. Examples include 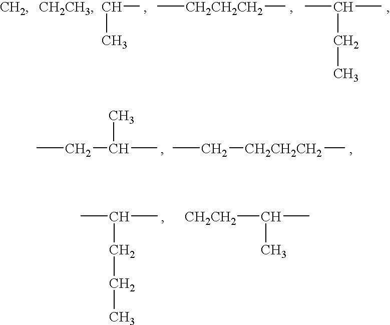
and the like.

Similarly C₀-C₄ alkyl refers to a covalent bond (C₀) or alkyl group as defined herein.

Unless otherwise indicated, as used herein, the term “cycloalkyl” includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl. “Bicycloalkyl” and “tricycloalkyl” groups are non-aromatic saturated cyclic alkyl moieties consisting of two or three rings respectively, wherein said rings share at least one carbon atom. For purposes of the present invention, and unless otherwise indicated, a bicycloalkyl group is a cycloalkyl group that contains two rings and containing 10 to 14 ring carbon atoms; it includes spiro groups and fused ring groups. Examples of bicycloalkyl groups include, but are not limited to, bicyclo-[3.1.0]-hexyl, bicyclo-2.2.1]-hept-1-yl, norbornyl, spiro[4.5]decyl, spiro[4.4]nonyl, spiro[4.3]octyl, and spiro[4.2]heptyl, and as defined herein, tricyclocylcoalkyl groups is a cycloalkyl group containing three rings and containing 10 to 14 ring carbon atoms. They may contain all fused rings, all spiro rings or a combination thereof. An example of a tricycloalkyl group is adamantanyl. Other cycloalkyl, bicycloalkyl, and tricycloalkyl groups are known in the art, and such groups are encompassed by the definitions “cycloalkyl”, “bicycloalkyl” and “tricycloalkyl” herein.

“Cycloalkenyl”, “bicycloalkenyl”, and “tricycloalkenyl” refer to non-aromatic each cycloalkyl, bicycloalkyl, and tricycloalkyl moieties as defined above, except that they each include one or more carbon-carbon double bonds connecting carbon ring members (an “endocyclic” double bond) and/or one or more carbon-carbon double bonds connecting a carbon ring member and an adjacent non-ring carbon (an “exocyclic” double bond). Examples of cycloalkenyl groups include, but are not limited to, cyclopentenyl, cyclobutenyl, and cyclohexenyl. A non-limiting example of a bicycloalkenyl group is norbornenyl. Cycloalkyl, cycloalkenyl, bicycloalkyl, and bicycloalkenyl groups also include groups that are substituted with one or more oxo moieties. Examples of such groups with oxo moieties are oxocyclopentyl, oxocyclobutyl, oxocyclopentenyl and norcamphoryl. Other cycloalkenyl, bicycloalkenyl, and tricycloalkenyl groups are known in the art, and such groups are included within the definitions “cycloalkenyl”, “bicycloalkenyl” and “tricycloalkenyl” herein.

Unless otherwise indicated, as used herein, the term ‘aryl’ includes an organic radical containing only carbon ring atoms derived from an aromatic hydrocarbon, such as phenyl (Ph), naphthyl, indenyl, indanyl, 1,2,3,4-tetrahydro-naphthalenyl, (6,7,8,9-tetrahydro-5H-benzocyclohepten-yl, 2-[(2,3-dihydrobenzofuran-6-ylmethyl), and fluorenyl, except that one hydrogen is removed. “Aryl” encompasses fused ring groups wherein at least one ring is aromatic and the other ring attacahed to can be either in the aromatic or non-aromatic ring of the “Aryl” group. However, the term “aryl” excludes “heteroaryl”, as defined herein. The most preferred aryl is phenyl.

Unless otherwise indicated, as used herein, the terms “heterocyclic” and “heterocycloalkyl” are synonymous and are used interchangeably. They both refer to non-aromatic cyclic groups containing one or more ring heteroatoms, preferably from one to four heteroatoms, each selected from O, S and N and more preferably containing 1, 2, or 3 ring heteroatoms, and more preferably containing 1 or 2 ring heteroatoms. It includes structures containing 1 ring or more than 1 ring. A “heterobicycloalkyl” group is a type of heterocyclic alkyl group; it is a non-aromatic two-ringed cyclic groups, containing 10 to 14 ring atoms wherein said rings share one or two atoms, and wherein at least one of the ring atoms is a heteroatom (O, S, or N). Unless otherwise indicated, for purposes of the present invention, heterobicycloalkyl groups include spiro groups and fused ring groups. In one embodiment, each ring in the heterobicycloalkyl contains up to four heteroatoms (i.e. from zero to four heteroatoms, provided that at least one ring contains at least one heteroatom). The heterocyclic groups of this invention can also include ring systems substituted with one or more oxo moieties. Examples of non-aromatic heterocyclic groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

Unless otherwise indicated, as used herein, “heteroaryl” refers to aromatic groups containing one or more ring heteroatoms, preferably from one to four heteroatoms, and more preferably 1-3 ring heteroatoms and most preferably 1 or 2 ring heteroatoms selected from O, S and N. The term also includes a multicyclic group containing one or more ring heteroatoms wherein at least one ring of the group is heteroaromatic. The heteroaryl groups of this invention can also include ring systems substituted with one or more oxo moieties, wherein the tautomers of such oxo substituted rings are heteroaryl, as defined hereinabove. Examples of heteroaryl groups are pyridinyl, pyridazinyl, imidazolyl, pyrimidinyl, pyrazolyl, triazolyl, pyrazinyl, quinolyl, isoquinolyl, 1,2,3,4-tetrahydroquinolyl, tetrazolyl, furyl, thienyl, isoxazolyl, thiazolyl, oxazolyl, isothiazolyl, pyrrolyl, indolyl, benzimidazolyl, benzofuranyl, cinnolinyl, indazolyl, indolizinyl, phthalazinyl, triazinyl, 1,2,4-trizainyl, 1,3,5-triazinyl, isoindolyl, 1-oxoisoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, and azaindolyl.

Unless otherwise indicated, as used herein, the term “cycloalkoxy”, means “cycloalkyl-O-”, wherein “cycloalkyl” is as defined above.

Unless otherwise indicated, as used herein, the term “aryloxy”, means “aryl-O-”, wherein “aryl” is as defined above.

Unless otherwise indicated, as used herein, the term “heterocycloalkoxy”, means “heterocycloalkyl-O-”, wherein “heterocycloalkyl” is as defined above.

Unless otherwise indicated, as used herein, the term “heteroaryloxy”, means “heteroaryl-O-”, wherein “heteroaryl” is as defined above.

Unless otherwise indicated, as used herein, the term “prodrug” has its ordinary meaning. More specifically, it refers to a chemical compound converted into the active curative form by metabolic processes within the patient, e.g., mammal, especially human. For example, R² of Formula I may be inter alia OC(═O)R⁴ or OC(═O)OR⁴, in a prodrug embodiment; OC(═O)R⁴ or OC(═O)OR⁴ may be a prodrug of the corresponding OH of R² meaning that R² of OC(═O)R⁴ or OC(═O)OR⁴ may hydrolyze in vivo to form a more active component of the parent drug wherein R is OH.

The foregoing groups, as derived from the compounds listed above, may be C-attached or N-attached where such is possible. For instance, a group derived from pyrrole may be pyrrol-1-yl (N-attached) or pyrrol-3-yl (C-attached). The terms referring to the groups also encompass all possible tautomers. An embodiment of the present invention relates to compounds of Formula I wherein Z is (C═O) CHR¹R², wherein R¹ and R² are as defined herein.

In another aspect, the present invention relates to compounds of Formula I wherein Z is —C(═O)CHR¹R² and R is —H, —OH or —OC(═O)CH₃.

In another aspect, Z is —C(═O)C(═O)R¹.

In another embodiment of the invention Z is R¹, as defined hereinabove.

In another aspect, the present invention relates to compounds of the Formula I wherein
R¹ is selected from —C₁-C₂₀ alkyl, —C₂-C₂₀ alkenyl, —C₂-C₂₀ alkynyl, —C₃-C₂₀ cycloalkyl, -(4-20 membered)heterocycloalkyl, —C₅-C₂₀ bicycloalkyl, —C₆-C₂₀ aryl and -(5-20 membered)heteroaryl.

In an embodiment thereof, R¹ is C₃-C₁₀ cycloalkyl, wherein R¹ optioanlly contains one to two double or triple bonds.

In another embodiment, R¹ is C₁-C₁₂ alkyl, C₃-C₁₁ cycloalkyl, (3-11) membered heterocycloalkyl, C₆-C₁₀ aryl, or (5-10 membered)heteroaryl. It is preferred that R¹ is C₁-C₄ alkyl. In this embodiment, R¹, especially when it is C₁-C₄ alkyl, is substituted with one to two substituents independently selected from F, OH, O(C═O)Me, (C₆-C₁₀)aryl and (5-10 membered) heteroaryl. In a further aspect, the preferred cyclalkyl and cyclalkenyl moieties are C₃-C₈ monocycloalkyl, C₅-C₁₁ bi or tri cycloalkyl, C₅-C₈ cyclomonoalkenyl and C₇-C₁₁ bi or tricycloalkenyl. In another embodiment, R¹ is C₅-C₁₁ bicycloalkyl, C₅-C₁₁ tricycloalkyl (5-11 membered)heterobicycloalkyl, aryl or heteroaryl. As defined herein, aryl and heteroaryl includes moieties containing two or three rings wherein one ring is completely aromatic or heteroaromatic, respectively and the other ring(s) is partially unsaturated and a third ring, if present is partially or fully saturated or completely aromatic or heteroaromatic. A preferred embodiment of R¹ is 1,2,3,4-tetrahydronapthalen-2-yl, indan-2-yl, 2-(6,7,8,9-tetrahydro)₅H-benzocyclo-hepten-6-yl, 2-(decahydro-naphthalene-2-yl, or 2-(2,3-dihydrobenzofuran-6-yl methyl.

In another aspect, R¹ is —C₃-C₇ cycloalkyl, e.g., [2.2.1]-heptanyl.

In another aspect, R¹ is selected from —C₁-C₁₀ alkyl, —C₂-C₁₀ alkenyl, —C₃-C₁₀ cycloalkyl, phenyl, thienyl and pyridyl, wherein R¹ is optionally independently substituted with from one to two substituents independently selected from —C₁-C₄ alkyl, —C₁-C₄ alkoxy, —F, —Cl, —Br, —CF₃, phenyl and phenoxy.

In another aspect, R¹ is selected from phenyl, thienyl, and pyridyl, wherein R¹ is optionally independently substituted with from one to two substituents independently selected from —F, —Cl, —CH₃, —CF₃, phenyl and phenoxy.

In another aspect, R¹ is —C₁-C₁₀ alkyl, wherein R¹ optionally contains one to two double or triple bonds.

In another aspect, R¹ is aryl especially phenyl or heteroaryl or alkyl wherein the R¹ group is unsubstituted or substituted with halo or R^{1a} groups. It is preferred that when R¹ is alkyl, aryl or heteroaryl, the R¹ groups are unsubstitued or substituted with one or more hydroxy, halo, aryloxy, heteroaryloxy, aryl, heteroaryl, or heterocyclic, which aryloxy, heteroaryloxy, aryl, heteroaryl or heterocyclic group may be unsubstituted or substituted with one to three R^{1b} s, especially halo, hydroxy and alkyl. In an aspect of the present invention R¹ is substituted by one or more halo groups, especially fluorine.

In another aspect, R¹ is C₃-C₁₀ cycloalkyl, wherein R¹ optionally contains one to two double or triple bonds.

In another aspect, R¹ is C₅-C₁₅ bicycloalkyl, wherein R¹ optionally contains one to two double or triple bonds.

In another aspect, the present invention relates to compounds of the Formula I wherein R² is selected from —H, —OH and —OC(═O)CH₃.

In another aspect, R² is selected from —H and —OH.

In another aspect, the present invention relates to compounds of the Formula I wherein R³ is selected from —C₁-C₄ alkyl, allyl and —CH₂CH₂SCH₃. It is preferred that R³ is C₁-C₄ alkyl.

In another aspect, R³ is selected from Me, Et, n-Pr, i-Pr, n-Bu, i-Bu, s-Bu, t-Bu allyl, and —CH₂CH₂SCH₃.

In another aspect, the present invention relates to compounds of the Formula I wherein R⁵ is —H.

In an aspect of the present invention, it is preferred that U, W, Y and X are all CH or that at most 2 of U, W, Y and X are N. In a more preferred embodiment, at most only one of U, W, Y and X are N, the remainder being all CH; that is the heteroaryl group containing X, Y, W and U contains at most only 1 or 2 ring nitrogen atoms. If the ring contains more than two nitrogen atoms, no more than two of the nitrogen ring atoms are adjacent. Examples include, but are not limited to pyridyl, pyrimidyl, pyrazoyl, pyridizinyl, quinolinyl, quinazolinyl, quinoxalinyl, cyclopenta pyrimidine and dihydropyrrolo pyrimidine, and the like.

In another aspect, the present invention relates to compounds of the Formula I wherein R⁷ is selected from —H, —C₁-C₁₂ alkyl, —C₂-C₁₂ alkenyl, —C₁-C₂₀ alkoxy, —F, —Cl, —Br, —I, —CN, —NO₂, —C₃-C₁₅ cycloalkyl, -(3-15 membered)heterocycloalkyl, —C₆-C₁₅ aryl, -(5-15 membered) heteroaryl, —CHO, —C(═O)(C₁-C₁₅ alkyl), —C(═O)((5-15 membered)heterocycloalkyl), —C(═O)(C₅-C₁₅ aryl), —C(═O)((5-15 membered)heteroaryl), —C(═O)(C₅-C₁₅ cycloalkyl), —C(═O)O(C₁-C₈ alkyl), —C(═O)N(C₁-C₁₀ alkyl)(C₁-C₁₀ alkyl), —C(═O)N(C_{zero}-C₁₀ alkyl)(C₆-C₁₀ aryl), —C(═O)N(C_{zero}-C₁₀ alkyl)((5-10 membered)heteroaryl), —C(═O)N(C_{zero}-C₁₀ alkyl)((5-10 membered) heterocycloalkyl), —C(═O)N(C_{zero}-C₁₀ alkyl)(C₅-C₁₀ cycloalkyl), —SO₂—(C₁-C₆ alkyl), —SO₂—(C₃-C₈ cycloalkyl), —SO₂—(C₆-C₁₀ aryl), —SO₂-((5-10 membered)heteroaryl), wherein said alkyl, cycloalkyl, heterocycloalkyl, aryl and heteroaryl are each optionally independently substituted with from one to three substituents independently selected from —F, —Cl, —Br, —I, —OH, —C₁-C₆ alkoxy, —C₂-C₆ alkenoxy, —C₂-C₆ alkynoxy, —NR⁹R¹⁰, —(C₁-C₁₁ alkyl)-NR⁹R¹⁰, —C(═O)R¹¹, —SO₂—R¹¹, —C(═O)OR¹², —C(═O)NR⁹R¹⁰, —SO₂—NR⁹R¹⁰—C₃-C₁₅ cycloalkyl, -(4-15 membered) heterocycloalkyl, —C₆-C₁₅ aryl, -(5-15 membered)heteroaryl, -(4-12 membered) heterocycloalkoxy, —C₆-C₁₂ aryloxy and -(6-12 membered)heteroaryloxy.

In another aspect, R⁷ is selected from —C₁-C₁₂ alkyl, —C₂-C₁₂ alkenyl, —C₃-C₁₅ cycloalkyl and -(4-15 membered)heterocycloalkyl, wherein said alkyl, alkenyl, cycloalkyl and heterocycloalkyl are each optionally independently substituted with from one to three substituents independently selected from —OH, —C₁-C₆ alkoxy, —C₂-C₆ alkenoxy, —C₂-C₆ alkynoxy, —NR⁹R¹⁰ and —(C₁-C₇ alkyl)-NR⁹R¹⁰.

In another aspect, R⁷ is selected from —C₁-C₁₂ alkyl, —C₂-C₁₂ alkenyl, —C₃-C₁₅ cycloalkyl and 4-15 membered)heterocycloalkyl, wherein said alkyl, alkenyl, cycloalkyl and heterocycloalkyl are each optionally independently substituted with from one to three substituents independently selected from —OH, —C₁-C₆ alkoxy, —C₂-C₆ alkenoxy and —C₂-C₆ alkynoxy.

In another aspect, R⁷ is selected from —C₁-C₁₂ alkyl, —C₂-C₁₂-alkenyl and —C₃-C₁₅ cycloalkyl, wherein said alkyl, alkenyl and cycloalkyl are each optionally independently substituted with from one to three substituents NR⁹R¹⁰.

In another aspect, R⁷ is a -(4-15 membered)heterocycloalkyl, wherein said heterocycloalkyl is optionally substituted with from one to three substituents independently selected from —OH, —C₁-C₆ alkyl, —C₂-C₆ alkenyl, —C₂-C₆ alkynyl, —C₁-C₆ alkoxy, —C₂-C₆ alkenoxy, —C₂-C₆ alkynoxy, —C₆-C₁₀ aryl and -(5-15 membered)heteroaryl.

If R⁷ is OH, and attached to a carbon atom, it is to be understood that the compounds of the present invention include tautomers wherein the OH group is tautomerized to the corresponding C═O groups. It is to be noted that when m is O, then the ring containing X, Y, W and U has only one substituent, i.e.,

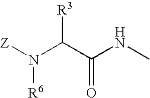

It is preferred that R⁷ is:
(a) hydrogen;
(b) aryloxy wherein the aryl group is unsubstituted or substituted with R^{1a} or halo, especially F;
(c) alkyl, which is unsubstituted or substituted with R^{1a} or halo, especially hydroxyalkyl or one or more fluoro, (C═O)R⁷, (C═O)OR¹², heterocyclic groups such as morpholinyl, piperidinyl, and the like, NR⁹R¹⁰, when R⁹ and R¹⁰ are independently hydrogen alkyl groups, or cycloalkyl, cycloalkyl alkyl groups, aryl, arylalkyl, heterocyclic or heterocyclic alkyl, especially wherein heterocyclic is tetrahydropyrrole, hydroxyalkyl, mono, di- or trihalo fluoro, which R⁹ and R¹⁰ groups themselves may be unsubstituted or substituted with R^{1a};
(d) aryl alkyl whrefein the aryl alkyl group is substituted with R^{1a};
(e) heterocyclic group selected from piperazinyl which is optionally substituted with R^{1a} or (C═O)OR¹²;
(f) alkenyl groups, substituted with R^{1a}, especially (C═O)R¹¹; and
(g) NR⁹R⁰.

In another aspect, NR⁹R¹⁰ is selected from —N(C_{zero}-C₆ alkyl)(C_{zero}-C₁₂ alkyl), —N(C_{zero}-C₆ alkyl)(C₃-C₁₂ cycloalkyl), —N(C₃-C₆ cycloalkyl)(C₃-C₁₂ cycloalkyl) and —N(C_{zero}-C₆ alkyl)((3-12 membered)heterocycloalkyl), wherein said NR⁹R¹⁰ may optionally be substituted with from one to six fluorine atoms or with from one to three substituents independently selected from —OH, —NH₂, —NH(C₁-C₄ alkyl), —C₁-C₆ alkoxy, —C₂-C₆ alkenoxy and —C₂-C₆ alkynoxy, and wherein said NR⁹R¹⁰ may optionally contain one to three double or triple bonds.

A preferred embodiment of the present invention is directed to compounds of the formula I wherein Z is (C═O)CHR¹R², R² is H or OH, R¹ is C₁-C₂₀ alkyl, aryl, heteroaryl, which R¹ group is unsubstittued or substituted with one or more R^{1a} groups as defined herein. Preferably, R^{1a} is OH, alkyl, alkoxy, halo, NR⁹R¹⁰, C(O)NR⁹R¹⁰, (C═O)OR¹¹, cycloalkyl, heterocyclic, heteroaryl, aryloxy or heteroaryloxy, wherein the cycloalkyl, heterocycloalkyl, aryl, heteroaryl, aryloxy and heteraryloxy are unsubstituted or substituted with one to three R^{1b} groups wherein R^{1b} preferably is OH, alkyl, alkoxy, halo, NR⁹R¹⁰, (C═O)NR⁹R¹⁰, (C═O)R¹¹ aryloxy or heteroaryloxy, wherein said alkyl, aryloxy, or heteroaryloxy group is unsubsttitued or substituted from 1-6 fluorine atoms or one to two substituents, selected from alkoxy or hydroxy;
R⁹ and R¹⁰ are independently H, alkyl, CF³, (C═O)R¹¹, —C═(O)OR², —C═(O)NR¹¹R¹², (C₀-C₄ alkylene), C₃-C₂₀ cycloalkyl, (C₀-C₄ alkylene)(5-10 membered heterocycloalkyl), (C₀-C₄ alkylene) C₆-C₁₀ aryl and (C₀-C₄ alkyl)(5-10 membered heteroaryl);
R¹¹ and R¹² are independently hydrogen, C₁-C₅ alkyl, (C₀-C₄ alkylene)C₃-C₁₅Cycloalkyl, (C₀-C₄ alkylene C₆-C₁₅ aryl), C₀-C₄ alkylene((5-15 membered heterocyclic and C₀-C₄ alkylene ((5-membered)heteroaryl wherein R¹¹ and R¹² are unsubstituted or substituted from one to three substituents independenetly selected from R^{1b}.
R³ is selected from C₁-C₆ alkyl, and C₀-C₄ alkylene C₃-C₆ cycloalkyl wherein said R³ group is unsubstituted or substituted with C₁-C₄ alkoxy or OH.

R⁵ is H or C₁-C₄ alkyl;
R⁷ is selected from —H, —C₁-C₂₀ alkyl, —C₂-C₂₀ alkenyl, —C₂-C₂₀ alkynyl, —C₁-C₂₀ alkoxy, —C₂-C₂₀ alkenoxy, —C₂-C₂₀ alkynoxy, —F, —Cl, —Br, —I, —CN, —NO₂, —OH, —CF₃, —NR⁹R¹⁰, (C₆-C₈)aryloxy, —(C₁-C₁, alkylene)-NR⁹R¹⁰, —C(═O)NR⁹R¹⁰, —C(═O)R¹¹, —CHO, —SO₂—R¹¹, —C(═O)OR¹², —(C_{zero}-C₄ alkylene)-(C₃-C₂₀ cycloalkyl), C_{zero}-C₄ alkylene)-(C₄-C₂₀ cycloalkenyl), —(C_{zero}-C₄ alkylene)-((3-20 membered)heterocycloalkyl), C_{zero}-C₄ alkylene)-(C₆-C₁₅ aryl) and —(C_{zero}-C₄ alkylene)-((5-15 membered)heteroaryl), wherein said heterocycloalkyl optionally contains from one to four ring double or triple bonds;
wherein R⁷ is optionally substituted with from one to six fluorine atoms or with from one to three substituents independently selected from the group R^{1a};
R⁸ is H or C₁-C₆ alkyl;
or a pharmaceutically acceptable salt thereof.

Another aspect of the present invention is directed to compounds of Formula I hereinabove, wherein
Z is C(═O)CHR¹R²;
   R¹ is C₁-C₂₀ alkyl, C₃-C₂₀ cycloalkyl, C₆-C₂₀ aryl, C₂-C₂₀ alkenyl, C₂-C₂₀ alkynyl, 4-20 membered heterocycloalkyl or 5-20 membered heteroaryl; said R¹ being unsubstituted or substituted by one to six fluorine atoms or with from one to three substituent independently selected from the group R^{1a};
   R^{1a} in each instance is independently selected from —Cl, —Br, —I, —CN, —NO₂, —NR⁹R¹⁰, —C(═O)NR⁹R¹⁰, —SO₂—NR⁹R¹⁰, —C(═O)R¹¹, —SO₂—R¹¹, —C(═O)OR¹², —C₃-C₁₅ cycloalkyl, —C₄-C₁₅ cycloalkenyl, -(4-20 membered)heterocycloalkyl, —C₆-C₁₅ aryl, -(5-15 membered)heteroaryl, —C₆-C₁₅ aryloxy and -(5-15 membered)heteroaryloxy, wherein said cycloalkyl, cycloalkenyl, heterocycloalkyl, aryl, heteroaryl, aryloxy and heteroaryloxy of R^{1a} are each optionally independently substituted with from one to three substituents independently selected from the group R^{1b};
   R^{1b} is in each instance independently selected from —OH, —C₁-C₆ alkyl, —C₂-C₆ alkenyl, —C₂-C₆ alkynyl, —C₁-C₆ alkoxy, —C₂-C₆ alkenoxy, —C₂-C₆ alkynoxy, —C₁-C₆ hydroxyalkyl, —F, —Cl, —Br, —I, —CN, —NO₂, —NR⁹R¹⁰, —C(═O)NR⁹R¹⁰, —C(═O)R¹¹, —SO₂—R¹¹, —C₆-C₁₅ aryloxy and -(5-15 membered)heteroaryloxy, wherein said alkyl, alkenyl and alkynyl of R^{1b} are each optionally independently substituted with from one to six fluorine atoms or with from one to two substituents independently selected from —C₁-C₄ alkoxy, or with a hydroxy group;
   R⁹ and R¹⁰ are in each instance each independently selected from —H, —C₁-C₁₂ alkyl, —C₂-C₁₂ alkenyl, —C₂-C₁₂ alkynyl, CF₃, —C(═O)R¹¹, —SO₂—R¹¹, —C(═O)OR², —C(═O)NR¹¹R¹², —SO₂—NR¹R¹², —(C_{zero}-C₄ alkylene)-(C₃-C₂₀ cycloalkyl), —(C_{zero}-C₄ alkylene)-((5-10 membered) heterocycloalkyl), —(C_{zero}-C₄ alkylene)-(C₆-C₁₀ aryl) and —(C_{zero}-C₄ alkylene)-((5-10 membered) heteroaryl), wherein said alkyl, alkenyl and alkynyl of R⁹ and R¹⁰ are each optionally independently substituted with from one to six fluorine atoms or with from one to two substituents independently selected from —C₁-C₄ alkoxy, with a hydroxy group, or C(═O)OR¹², and wherein said cycloalkyl, cycloalkenyl, bi- or tricycloalkyl, bi- or tricycloalkenyl, heterocycloalkyl, aryl and heteroaryl are each optionally independently substituted with from one to three substituents independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —C₁-C₆ hydroxyalkyl, —F, —Cl, —Br, —I, —CN, —NO₂, —CF₃, —NH₂, —C(═O)NH₂, —SO₂—NH₂, —C(═O)H and —C(═O)OH, wherein said alkyl, alkenyl and alkynyl substituents are each optionally independently further substituted with from one to six fluorine atoms or with from one to two substituents independently selected from —C₁-C₄ alkoxy, or with a hydroxy group or aryl group;
   R¹¹ and R¹² are in each instance each independently selected from hydrogen, —C₁-C₁₅ alkyl, —C₂-C₆ alkenyl, —C₂-C₆ alkynyl, —(C_{zero}-C₄ alkylene)-(C₃-C₁₅ cycloalkyl), —(C_{zero}-C₄ alkylene)-(C₄-C₈ cycloalkenyl), —(C_{zero}-C₄ alkylene)-(C₆-C₁₅ aryl), —(C_{zero}-C₄ alkylene)-((5-15 membered) heterocycloalkyl) and C_{zero}-C₄ alkylene)-((5-15 membered)heteroaryl);
   wherein R¹¹ and R¹² are each optionally independently substituted with from one to three substituents independently selected from the group R^{1b};
   R² is selected from —H, —OH, —NH₂, —CH₂OH, —C(CH₃)₂OH, —C(CH₃)(CH₂CH₃)(OH), —C(OH)(C_{zero}-C₄ alkyl)(C_{zero}-C₄ alkyl);
   R³ is selected from —C₁-C₆ alkyl, —C₂-C₆ alkenyl, —C₂-C₆ alkynyl and Ce —C₄ alkylene)-(C₃-C₆ cycloalkyl), wherein when R³ is alkyl, alkenyl or alkynyl, R³ is optionally independently substituted with a substituent independently selected from —C₁-C₄ alkoxy, —OH and —S(C₁-C₄ alkyl);
   R⁵ is selected from —H, —C₁-C₄ alkyl, —C₂-C₄ alkenyl, —C₂-C₄ alkynyl, —C(═O)(C₁-C₄ alkyl), —C₆-C₁₀ aryl, -((5-20 membered)heteroaryl), —SO₂—(C₆-C₁₀ aryl), —SO₂-((5-20 membered) heteroaryl), —SO₂—CH₂—(C₆-C₂₀ aryl) and —SO₂—CH₂-((5-20 membered)heteroaryl); and

R⁷ is selected from —H, —C₁-C₂₀ alkyl, —C₂-C₂₀ alkenyl, —C₂-C₂₀ alkynyl, —C₁-C₂₀ alkoxy, —C₂-C₂₀ alkenoxy, —C₂-C₂₀ alkynoxy, —F, —Cl, —Br, —I, —CN, —NO₂, —OH, —CF₃, —NR⁹R¹⁰, —(C₁-C₁₁ alkylene)-NR⁹R¹⁰, —C(═O)NR⁹R¹⁰, —C(═O)R¹¹, —CHO, —SO₂—R¹¹, —C(═O)OR¹², COOH, —(C_{zero}-C₄ alkylene)-(C₃-C₂₀ cycloalkyl), C_{zero}-C₄ alkylene)-(C₄-C₂₀ cycloalkenyl), —(C_{zero}-C₄ alkylene)-((3-20 membered)heterocycloalkyl), C_{zero}-C₄ alkylene)-(C₆-C₁₅ aryl) and C_{zero}-C₄ alkylene)-((5-15 membered)heteroaryl), wherein said heterocycloalkyl optionally contains from one to four ring double or triple bonds;
wherein R⁷ is optionally substituted with from one to six fluorine atoms or with from one to three substituents independently selected from the group R^{1a};
X, Y, W and U are each independently selected from carbon and nitrogen, wherein the heteroaryl ring of Formula I that contains X, Y, W and U or the heteroaryl ring containing two R⁷ groups taken together with the carbon atoms on the heteroaryl ring of the Formula containing X, Y, W and U are pyridyl, pyrimidyl, pyrazinyl, pyridazinyl, quinolinyl, quinazolinyl, quinoxalinyl, cyclopentapyrimidine, or dihydropyrrole pyrimidine with the proviso that the heteroaryl ring of Formula I that contains X, Y, W and U may not contain more than three nitrogen atoms in the ring, and with the further proviso that no more than two nitrogen atoms in the heteroaryl ring of Formula I that contains X, Y, W and U may be directly adjacent to each other in the ring, and with the further proviso when R⁷ is —OH and R⁷ is attached to a carbon atom of the heteroaryl ring Formula I that contains X, Y, W and U, and m is 1, 2 or 3, then the —OH group of R⁷ that is attached to a carbon atom of the heteroaryl ring of Formula I may be tautomerizable to a C═O group;
or the pharmaceutically acceptable salts of such compounds.

It is more preferred that R¹ is C₆-C₂₀ aryl and preferably phenyl which may be substituted by 1-3 fluoro groups;
R^{1a} is, in each instance independently OH, C₁-C₁₂alkyl, C₁-C₆ alkoxy, C₂-C₁₂ alkenyl, C₂-C₆ alkenoxy, SO₂NR⁹R¹⁰, C(═O)R¹¹, —(C═O)OR¹², C₃-C₁₅ cycloalkyl, C₄-C₁₅ cycloalkyenyl, (4-20membered)heterocycloalkyl, C₆-C₁₅ aryl, (5-15membered)heteroaryl, or C₆-C₁₅ aryloxy;
R⁹ and R¹⁰ are in each instance independently selected from H, C₁-C₁₂alkyl, C₂-C₁₂ alkenyl, C₂-C₁₂ alkynyl, CF₃, C(═O)R¹, SO₂R¹¹, C(═O)R¹², C(═O)NR¹¹R¹², SO₂NR¹¹R¹², (C₀-C₄ alkylene)cycloalkyl, (C₀-C₄ alkylene) (5 to 10 membered heterocycloalkyl, or (C₀-C₄ alkylene)(C₆-C₁₀ aryl);
R¹¹ and R¹² are in each instance each independently C₁-C₁₅alkyl, C₂-C₆ alkenyl, C₂-C₆ alkynyl, (C₀-C₄ alkylene), C₃-C₁₅ cycloalkyl, (C₀-C₆ alkylene)C₆-C₁₅ aryl, (C₀-C₄ alkylene) (5-10 membered)heterocyclic and C₀-C₄ alkylene) (5-10 membered)heteroaryl;
R³ is hydrogen or alkyl;
R⁵ is H or alklyl;
R⁷ is H, C₁-C₂₀ alkyl, C₂-C₂₀ alkenyl, C₂-C₂₀ alkynyl, C₁-C₂₀ alkoxy, C₂-C₂₀ alkenyloxy, C₂-C₂₀ alkynoxy, halo, CN, NO₂, OH, CF₃; C₆-C₁₈ aryloxy, C₁-C₁₁ alkylene, NR⁹R¹⁰, C(═O)NR⁹R¹⁰, C(═O)R¹¹, CHO, SO₂R¹¹, C(═O)OR¹², C₀-C₄ alkylene, cycloalkyl, C₀-C₄ alkylene, C₄-C₂₀ (cycloalkenyl), (C₀-C₄ alkylene) C₃-C₂₀ membered heterocyclic alkyl, (C₀-C₄ alkylene)(C₆-C₁₅ aryl) and (C₀-C₄ alkylene) (5-10 membered)heteroaryl where R² is optioanlly substuted with form one to six fluorine atoms or 1-3 R^{1a} groups and m is 0-3.

In another aspect of the present invention is directed to the above-identified compounds wherein Z is (C═O)CHR¹R², m is 0-3, R¹ is aryl or aralkyl, which mya be unsubstituted or substituted with one to six fluorine atoms or from one to three subsituents of R^{1a}; R⁵ is H, R³ is H or OH, X, Y, W and U are independently CH or N; and R⁷ is as defined hereinabove.

It is preferred that R¹ is alkyl substituted by phenyl, which ring is unsubstituted or substituted with halo; R⁵ is H; R³ is H; X, Y, W, and U are independently CH or N, and R⁷ is as defined hereinabove.

In another embodiment the preferred ring containing X, Y, W and U are pipridyl, pyrazinyl, pyridazinyl, quinolinyl, quinazolinyl, quinoxalinyl, cyclopentapyrimidine or dihydropyrrolopyrimidine.

In another embodiment R⁷ is H, alkyl, NR⁹R¹⁰ or alkenyl, which alkyl and alkenyl groups are unsubstituted or substituted by 1-3, R^{1a} groups and more preferably one R^{1a} group and R^{1a} is OH, aryl, heterocyclicalkyl, C(═O)R¹¹, NR⁹R¹⁰, or C═(O)OR¹²; R¹¹ is H or C₁-C₆ alkyl and R¹² is C₁-C₆ alkyl;
R⁹ is hydrogen or C₁-C₆ alkyl and R¹⁰ is C₁-C₆ alkyl, (C₀-C₄ alkylene)(C₆-C₁₀ aryl), or (C₀-C₄ alkylene) 5 or 6 membered heterocycloalkyl, wherein said alkyl or heterocycloalkyl group is unsubstituted or substituted with one to six fluorine atoms or up to 1 to 3 substituents selected from alkyl, aryl, C₀-C₄ alkylene (C₆-C₁₀ aryl), (C₀-C₄ alkylene) (C₃-C₆ cycloalkyl), C═(O)R¹², OH, and said aryl group is substituted with 1 to 3 substituents selected from halo, OH, alkyl, aryloxy, hydroxyalkyl or alkoxy or CF₃ It is preferred that aryl is phenyl. The preferred alkyl group in any of the definitions herein is 1-4 carbon atoms.

Specific embodiments of the present invention include the following compounds of Formula I, all pharmaceutically acceptable salts thereof, complexes thereof, and derivatives thereof that convert into a pharmaceutically active compound upon administration:
2-[2-(3,5-Difluoro-phenyl)-acetylamino]-N-pyridin-2-yl-butyramide
N-(5-Bromo-pyridin-2-yl)-2-[2-(3,5-difluoro-phenyl)-acetylamino]-butyramide
2-[2-(3,5-Difluoro-phenyl)-acetylamino]-N-(5-iodo-pyridin-2-yl)-butyramide
2-[2-(3-phenoxy-phenyl)-acetylamino]-pentanoic acid pyrazin-2-ylamide
2-[2-(3-phenoxy-phenyl)-acetylamino]-pentanoic acid pyrimidin-2-ylamide
2-[2-(3-phenoxy-phenyl)-acetylamino]-pentanoic acid (6-chloro-pyridazin-3-yl)-amide
2-[2-(3-phenoxy-phenyl)-acetylamino]-pentanoic acid pyrimidin-4-ylamide
2-[2-(3-phenoxy-phenyl)-acetylamino]-pentanoic acid (4-methyl-pyrimidin-2-yl)-amide
2-[2-(5-Bromo-pyridin-3-yl)-acetylamino]-pentanoic acid pyrazin-2-ylamide
2-(2-Hydroxy-3-methyl-butyrylamino)-pentanoic acid pyrazin-2-ylamide
2-[2-(3-phenoxy-phenyl)-acetylamino]-pentanoic acid (4-chloro-6-methyl-pyrimidin-2-yl)-amide
2-[2-(3-phenoxy-phenyl)-acetylamino]-pentanoic acid (6-chloro-pyrazin-2-yl)-amide
2-[2-(3,5-Difluoro-phenyl)-acetylamino]-N-pyrazin-2-yl-butyramide
2-[2-(3,5-Difluoro-phenyl)-acetylamino]-pentanoic acid pyrazin-2-ylamide
2-(2-Hydroxy-3,3-dimethyl-butyrylamino)-pentanoic acid pyrazin-2-ylamide
2-[2-(3-phenoxy-phenyl)-acetylamino]-N-pyrazin-2-yl-propionamide
2-[2-(3,5-Difluoro-phenyl)-2-hydroxy-acetylamino]-pentanoic acid pyrazin-2-ylamide
2-[2-(3,5-Difluoro-phenyl)-2-hydroxy-acetylamino]-pentanoic acid pyrazin-2-ylamide
2-[2-(3-phenoxy-phenyl)-acetylamino]-pentanoic acid (6-dibutylamino-pyrazin-2-yl)-amide
2-[2-(3-phenoxy-phenyl)-acetylamino]-pentanoic acid [6-(1-ethyl-propylamino)-pyrazin-2-yl]-amide
2-[2-(3-phenoxy-phenyl)-acetylamino]-pentanoic acid (6-isopropylamino-pyrazin-2-yl)-amide
2-[2-(3-phenoxy-phenyl)-acetylamino]-pentanoic acid (6-ethylamino-pyrazin-2-yl)-amide
2-[2-(3-phenoxy-phenyl)-acetylamino]-pentanoic acid (6-butylamino-pyrazin-2-yl)-amide
2-[2-(3,5-Difluoro-phenyl)-acetylamino]-pentanoic acid (6-chloro-pyridazin-3-yl)-amide
2-[2-(3,5-Difluoro-phenyl)-acetylamino]-pentanoic acid pyrimidin-4-ylamide
2-[2-(3-phenoxy-phenyl)-acetylamino]-pentanoic acid [6-(butyl-methyl-amino)-pyrazin-2-yl]-amide
2-[2-(3,5-Difluoro-phenyl)-acetylamino]-pentanoic acid (5-trifluoromethyl-pyridin-2-yl)-amide
6-{2-[2-(3,5-Difluoro-phenyl)-acetylamino]-pentanoylamino}-nicotinamide
2-[2-(3,5-Difluoro-phenyl)-acetylamino]-pentanoic acid [5-(4-chloro-phenoxy)-pyrimidin-2-yl]-amide
2-[2-(3,5-Difluoro-phenyl)-acetylamino]-pentanoic acid (5-o-tolyloxy-pyrimidin-2-yl)-amide
2-[2-(3,5-Difluoro-phenyl)-acetylamino]-pentanoic acid (5-m-tolyloxy-pyrimidin-2-yl)-amide
2-[2-(3,5-Difluoro-phenyl)-acetylamino]-pentanoic acid [5-(3-tert-butyl-phenoxy)-pyrimidin-2-yl]-amide
2-[2-(3,5-Difluoro-phenyl)-acetylamino]-pentanoic acid (5-pentyloxy-pyrimidin-2-yl)-amide
2-[2-(3,5-Difluoro-phenyl)-acetylamino]-pentanoic acid [5-(3-trifluoromethyl-phenoxy)-pyrimidin-2-yl]-amide
2-[2-(3,5-Difluoro-phenyl)-acetylamino]-pentanoic acid [5-(naphthalen-2-yloxy)-pyrimidin-2-yl]-amide
2-[2-(3,5-Difluoro-phenyl)-acetylamino]-pentanoic acid [5-(4-methoxy-phenoxy)-pyrimidin-2-yl]-amide
2-[2-(3,5-Difluoro-phenyl)-acetylamino]-pentanoic acid (5-p-tolyloxy-pyrimidin-2-yl)-amide
2-[2-(3,5-Difluoro-phenyl)-acetylamino]-pentanoic acid (5-methyl-pyrimidin-2-yl)-amide
2-[2-(3,5-Difluoro-phenyl)-acetylamino]-pentanoic acid (5-bromo-pyrimidin-2-yl)-amide
2-[2-(3,5-Difluoro-phenyl)-acetylamino]-pentanoic acid (5-chloro-pyrimidin-2-yl)-amide
6-{2-[2-(3,5-Difluoro-phenyl)-acetylamino]-pentanoylamino}-nicotinic acid ethyl ester
3-(6-{2-[2-(3,5-Difluoro-phenyl)-acetylamino]-pentanoylamino}-pyridin-3-yl)-but-2-enoic acid methyl ester
2-[2-(3,5-Difluoro-phenyl)-acetylamino]-pentanoic acid (5-benzyl-pyridin-2-yl)-amide
4-(6-{2-[2-(3,5-Difluoro-phenyl)-acetylamino]-pentanoylamino}-pyridin-3-yl)-piperazine-1-carboxylic acid tert-butyl ester
2-[2-(3,5-Difluoro-phenyl)-acetylamino]-pentanoic acid (5-cyano-pyridin-2-yl)-amide
3-(6-{2-[2-(3,5-Difluoro-phenyl)-acetylamino]-pentanoylamino}-pyridin-3-yl)-butyric acid methyl ester
2-[2-(3,5-Difluoro-phenyl)-acetylamino]-pentanoic acid (6-chloro-pyridazin-3-yl)-amide
2-[2-(3,5-Difluoro-phenyl)-acetylamino]-pentanoic acid [5-(1-hydroxy-1-methyl-ethyl)-pyridin-2-yl]-amide
2-[2-(3,5-Difluoro-phenyl)-acetylamino]-pentanoic acid (5-bromo-pyrazin-2-yl)-amide
2-[2-(3,5-Difluoro-phenyl)-acetylamino]-pentanoic acid [5-(1-hydroxy-1-methyl-pentyl)-pyridin-2-yl]-amide
2-[2-(3,5-Difluoro-phenyl)-acetylamino]-pentanoic acid [5-(1-butyl-vinyl)-pyridin-2-yl]-amide
2-[2-(3,5-Difluoro-phenyl)-acetylamino]-pentanoic acid {5-[methyl-(3-methyl-butyl)-amino]-pyrazin-2-yl}-amide
2-[2-(3,5-Difluoro-phenyl)-acetylamino]-pentanoic acid [5-(butyl-methyl-amino)-pyrazin-2-yl]-amide
2-[2-(3,5-Difluoro-phenyl)-acetylamino]-pentanoic acid [5-(butyl-ethyl-amino)-pyrazin-2-yl]-amide
2-[2-(3,5-Difluoro-phenyl)-acetylamino]-pentanoic acid [5-(cyclopropylmethyl-propyl-amino)-pyrazin-2-yl]-amide
2-[2-(3,5-Difluoro-phenyl)-acetylamino]-pentanoic acid [5-(hexyl-methyl-amino)-pyrazin-2-yl]-amide
2-[2-(3,5-Difluoro-phenyl)-acetylamino]-pentanoic acid {6-[methyl-(3-methyl-butyl)-amino]-pyridazin-3-yl}-amide
2-[2-(3,5-Difluoro-phenyl)-acetylamino]-pentanoic acid [6-(butyl-methyl-amino)-pyridazin-3-yl]-amide
2-[2-(3,5-Difluoro-phenyl)-acetylamino]-pentanoic acid [6-(1-ethyl-propylamino)-pyridazin-3-yl]-amide
2-[2-(3,5-Difluoro-phenyl)-acetylamino]-pentanoic acid [6-(butyl-ethyl-amino)-pyridazin-3-yl]-amide
2-[2-(3,5-Difluoro-phenyl)-acetylamino]-pentanoic acid [6-(hexyl-methyl-amino)-pyridazin-3-yl]-amide
3-(2-{2-[2-(3,5-Difluoro-phenyl)-acetylamino]-pentanoylamino}-pyrimidin-5-yl)-but-2-enoic acid methyl ester
2-[2-(3,5-Difluoro-phenyl)-acetylamino]-pentanoic acid (5-bromo-pyridin-2-yl)-amide
2-[2-(3,5-Difluoro-phenyl)-acetylamino]-pentanoic acid (5-morpholin-4-yl-pyrazin-2-yl)-amide
2-[2-(3,5-Difluoro-phenyl)-acetylamino]-pentanoic acid (6-methyl-pyridin-2-yl)-amide
2-[2-(3,5-Difluoro-phenyl)-acetylamino]-pentanoic acid (5-phenethylamino-pyrazin-2-yl)-amide
2-[2-(3,5-Difluoro-phenyl)-acetylamino]-pentanoic acid [5-(benzyl-methyl-amino)-pyrazin-2-yl]-amide
2-[2-(3,5-Difluoro-phenyl)-acetylamino]-pentanoic acid (5-dibenzylamino-pyrazin-2-yl)-amide
2-[2-(3,5-Difluoro-phenyl)-acetylamino]-pentanoic acid [5-(1-methoxymethyl-propylamino)-pyrazin-2-yl]-amide
2-[2-(3,5-Difluoro-phenyl)-acetylamino]-pentanoic acid (5-isopropylamino-pyrazin-2-yl)-amide
2-[2-(3,5-Difluoro-phenyl)-acetylamino]-pentanoic acid [5-(2-hydroxy-ethylamino)-pyrazin-2-yl]-amide
2-[2-(3,5-Difluoro-phenyl)-acetylamino]-pentanoic acid (5-benzylamino-pyrazin-2-yl)-amide
2-[2-(3,5-Difluoro-phenyl)-acetylamino]-pentanoic acid [5-(benzyl-ethyl-amino)-pyrazin-2-yl]-amide
2-[2-(3,5-Difluoro-phenyl)-acetylamino]-pentanoic acid (5-pyrrolidin-1-yl-pyrazin-2-yl)-amide
2-[2-(3-Fluoro-5-pyrrolidin-1-yl-phenyl)-acetylamino]-pentanoic acid (5-pyrrolidin-1-yl-pyrazin-2-yl)-amide
2-[2-(3,5-Difluoro-phenyl)-acetylamino]-pentanoic acid pyrazin-2-ylamide
3-(6-{2-[2-(3,5-Difluoro-phenyl)-acetylamino]-pentanoylamino}-pyridin-3-yl)-acrylic acid methyl ester
2-[2-(3,5-Difluoro-phenyl)-acetylamino]-pentanoic acid [5-(1-methyl-3-oxo-but-1-enyl)-pyridin-2-yl]-amide
2-[2-(3,5-Difluoro-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid [5-(pyrazin-2-ylamino)-pyrazin-2-yl]-amide
2-[2-(3,5-Difluoro-phenyl)-acetylamino]-pentanoic acid [5-(1-hydroxy-1-methyl-ethyl)-pyrazin-2-yl]-amide
2-[2-(3,5-Difluoro-phenyl)-acetylamino]-pentanoic acid [5-(3-amino-1-methyl-butyl)-pyridin-2-yl]-amide
2-[2-(3,5-Difluoro-phenyl)-acetylamino]-pentanoic acid [5-(1-methyl-3-methylamino-butyl)-pyridin-2-yl]-amide
2-[2-(3,5-Difluoro-phenyl)-acetylamino]-pentanoic acid {5-[3-(2-hydroxy-ethylamino)-1-methyl-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid [5-(3-hydroxy-1-methyl-butyl)-pyridin-2-yl]-amide
2-[2-(3,5-Difluoro-phenyl)-acetylamino]-pentanoic acid {5-[3-(2,2,2-trifluoro-ethylamino)-butyl]-pyridin-2-yl}-amide
3-(5-{2-[2-(3,5-Difluoro-phenyl)-acetylamino]-pentanoylamino}-pyrazin-2-ylamino)-propionic acid methyl ester
2-[2-(3,5-Difluoro-phenyl)-acetylamino]-pentanoic acid (5-acetyl-pyridin-2-yl)-amide
2-[2-(3,5-Difluoro-phenyl)-acetylamino]-pentanoic acid [5-(3-oxo-but-1-enyl)-pyridin-2-yl]-amide
3-(6-{2-[2-(3,5-Difluoro-phenyl)-acetylamino]-pentanoylamino}-pyridin-3-yl)-propionic acid methyl ester
6-{2-[2-(3,5-Difluoro-phenyl)-acetylamino]-pentanoylamino}-nicotinic acid methyl ester
2-[2-(3,5-Difluoro-phenyl)-acetylamino]-pentanoic acid [5-(3-oxo-butyl)-pyridin-2-yl]-amide
2-[2-(3,5-Difluoro-phenyl)-acetylamino]-pentanoic acid [5-(1-hydroxy-ethyl)-pyridin-2-yl]-amide
2-[2-(3,5-Difluoro-phenyl)-acetylamino]-pentanoic acid [5-(3-methylamino-butyl)-pyridin-2-yl]-amide
2-[2-(3,5-Difluoro-phenyl)-acetylamino]-pentanoic acid [5-(3-ethylamino-butyl)-pyridin-2-yl]-amide
2-[2-(3,5-Difluoro-phenyl)-acetylamino]-pentanoic acid [5-(3-propylamino-butyl)-pyridin-2-yl]-amide
2-[2-(3,5-Difluoro-phenyl)-acetylamino]-pentanoic acid [5-(3-isopropylamino-butyl)-pyridin-2-yl]-amide
2-[2-(3,5-Difluoro-phenyl)-acetylamino]-pentanoic acid [5-(3-butylamino-butyl)-pyridin-2-yl]-amide
2-[2-(3,5-Difluoro-phenyl)-acetylamino]-pentanoic acid [5-(3-isobutylamino-butyl)-pyridin-2-yl]-amide
2-[2-(3,5-Difluoro-phenyl)-acetylamino]-pentanoic acid {5-[3-(3-methyl-butylamino)-butyl]-pyridin-2-yl}-amide
2-[2-(3,5-Difluoro-phenyl)-acetylamino]-pentanoic acid {5-[1-(3-methyl-butylamino)-ethyl]-pyridin-2-yl}-amide
6-{2-[2-(3,5-Difluoro-phenyl)-acetylamino]-pentanoylamino}-nicotinic acid
2-[2-(3,5-Difluoro-phenyl)-acetylamino]-pentanoic acid [5-(1-isobutylamino-ethyl)-pyridin-2-yl]-amide
2-[2-(3,5-Difluoro-phenyl)-acetylamino]-pentanoic acid {5-[1-(3,3-dimethyl-butylamino)-ethyl]-pyridin-2-yl}-amide
2-[2-(3,5-Difluoro-phenyl)-acetylamino]-pentanoic acid {5-[1-(1-ethyl-propylamino)-ethyl]-pyridin-2-yl}-amide
2-[2-(3,5-Difluoro-phenyl)-acetylamino]-pentanoic acid {5-[1-(2,2,2-trifluoro-ethylamino)-ethyl]-pyridin-2-yl}-amide
2-[2-(3,5-Difluoro-phenyl)-acetylamino]-pentanoic acid [5-(1-butylamino-ethyl)-pyridin-2-yl]-amide
2-[2-(3,5-Difluoro-phenyl)-acetylamino]-pentanoic acid [5-(1-morpholin-4-yl-ethyl)-pyridin-2-yl]-amide
2-[2-(3,5-Difluoro-phenyl)-acetylamino]-pentanoic acid [5-(1-benzylamino-ethyl)-pyridin-2-yl]-amide
2-[2-(3,5-Difluoro-phenyl)-acetylamino]-pentanoic acid [5-(1-cyclopropylamino-ethyl)-pyridin-2-yl]-amide
2-[2-(3,5-Difluoro-phenyl)-acetylamino]-pentanoic acid [5-(1-cyclopropylamino-ethyl)-pyridin-2-yl]-amide
2-[2-(3,5-Difluoro-phenyl)-acetylamino]-pentanoic acid {5-[1-(1-benzyl-pyrrolidin-3-ylamino)-ethyl]-pyridin-2-yl}-amide
2-[2-(3,5-Difluoro-phenyl)-acetylamino]-pentanoic acid {5-[1-(1-benzyl-pyrrolidin-3-ylamino)-ethyl]-pyridin-2-yl}-amide
2-[2-(3,5-Difluoro-phenyl)-acetylamino]-pentanoic acid [5-(1-cyclobutylamino-ethyl)-pyridin-2-yl]-amide
2-[2-(3,5-Difluoro-phenyl)-acetylamino]-pentanoic acid [5-(3-oxo-butyl)-pyrazin-2-yl]-amide
2-[2-(3,5-Difluoro-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid [5-(3-morpholin-4-yl-butyl)-pyridin-2-yl]-amide
2-[2-(3,5-Difluoro-phenyl)-acetylamino]-pentanoic acid [5-(3-pyrrolidin-1-yl-butyl)-pyridin-2-yl]-amide
2-[2-(3,5-Difluoro-phenyl)-acetylamino]-pentanoic acid [5-(3-benzylamino-butyl)-pyridin-2-yl]-amide
2-[2-(3,5-Difluoro-phenyl)-acetylamino]-pentanoic acid [5-(3-cyclopropylamino-butyl)-pyridin-2-yl]-amide
2-[2-(3,5-Difluoro-phenyl)-acetylamino]-pentanoic acid [5-(3-cyclobutylamino-butyl)-pyridin-2-yl]-amide
2-[2-(3,5-Difluoro-phenyl)-acetylamino]-pentanoic acid [5-(1-pyrrolidin-1-yl-ethyl)-pyridin-2-yl]-amide
2-[2-(3,5-Difluoro-phenyl)-acetylamino]-pentanoic acid {5-[1-(4-methyl-piperazin-1-yl)-ethyl]-pyridin-2-yl}-amide
2-[2-(3,5-Difluoro-phenyl)-acetylamino]-pentanoic acid {5-[1-(4-methyl-piperazin-1-yl)-ethyl]-pyridin-2-yl}-amide
2-[2-(3,5-Difluoro-phenyl)-acetylamino]-pentanoic acid {5-[1-(2-hydroxy-ethylamino)-ethyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid [5-(3-cyclopropylamino-butyl)-pyridin-2-yl]-amide
2-[2-(3,5-Difluoro-phenyl)-acetylamino]-pentanoic acid [5-(3-cyclopropylamino-butyl)-pyridin-2-yl]-amide
2-[2-(3,5-Difluoro-phenyl)-acetylamino]-pentanoic acid [5-(3-oxo-propenyl)-pyridin-2-yl]-amide
2-[2-(3,5-Difluoro-phenyl)-acetylamino]-pentanoic acid [5-(3-hydroxy-3-methyl-butyl)-pyridin-2-yl]-amide
2-[2-(3,5-Difluoro-phenyl)-acetylamino]-pentanoic acid (5-hydroxymethyl-pyridin-2-yl)-amide
2-[2-(3,5-Difluoro-phenyl)-acetylamino]-pentanoic acid [5-(3-cyclobutylamino-butyl)-pyridin-2-yl]-amide
2-[2-(3,5-Difluoro-phenyl)-acetylamino]-pentanoic acid [5-(3-cyclobutylamino-butyl)-pyridin-2-yl]-amide
2-[2-(3,5-Difluoro-phenyl)-acetylamino]-pentanoic acid [5-(3-benzylamino-1-methyl-butyl)-pyridin-2-yl]-amide
2-[2-(3,5-Difluoro-phenyl)-acetylamino]-pentanoic acid [5-(3-benzylamino-1-methyl-butyl)-pyridin-2-yl]-amide
2-[2-(3,5-Difluoro-phenyl)-acetylamino]-pentanoic acid {5-[3-(1-phenyl-ethylamino)-butyl]-pyridin-2-yl}-amide
2-[2-(3,5-Difluoro-phenyl)-acetylamino]-pentanoic acid {5-[3-(1-phenyl-ethylamino)-butyl]-pyridin-2-yl}-amide
2-[2-(3,5-Difluoro-phenyl)-acetylamino]-pentanoic acid {5-[3-(2-hydroxy-1-phenyl-ethylamino)-butyl]-pyridin-2-yl}-amide
2-[2-(3,5-Difluoro-phenyl)-acetylamino]-pentanoic acid {5-[3-(2-hydroxy-1-phenyl-ethylamino)-butyl]-pyridin-2-yl}-amide
2-[2-(3,5-Difluoro-phenyl)-acetylamino]-pentanoic acid {5-[3-(2-trifluoromethyl-benzylamino)-butyl]-pyridin-2-yl}-amide
2-[2-(3,5-Difluoro-phenyl)-acetylamino]-pentanoic acid {5-[3-(3-trifluoromethyl-benzylamino)-butyl]-pyridin-2-yl}-amide
2-[2-(3,5-Difluoro-phenyl)-acetylamino]-pentanoic acid {5-[3-(2,4-difluoro-benzylamino)-butyl]-pyridin-2-yl}-amide
2-[2-(3,5-Difluoro-phenyl)-acetylamino]-pentanoic acid {5-[3-(4-chloro-benzylamino)-butyl]-pyridin-2-yl}-amide
2-[2-(3,5-Difluoro-phenyl)-acetylamino]-pentanoic acid {5-[3-(4-methoxy-benzylamino)-butyl]-pyridin-2-yl}-amide
2-[2-(3,5-Difluoro-phenyl)-acetylamino]-pentanoic acid {5-[3-(2-fluoro-benzylamino)-butyl]-pyridin-2-yl}-amide
2-[2-(3,5-Difluoro-phenyl)-acetylamino]-pentanoic acid {5-[3-(3-chloro-benzylamino)-butyl]-pyridin-2-yl}-amide
2-[2-(3,5-Difluoro-phenyl)-acetylamino]-pentanoic acid {5-[3-(4-fluoro-benzylamino)-butyl]-pyridin-2-yl}-amide
2-[2-(3,5-Difluoro-phenyl)-acetylamino]-pentanoic acid {5-[3-(3-fluoro-benzylamino)-butyl]-pyridin-2-yl}-amide
2-[2-(3,5-Difluoro-phenyl)-acetylamino]-pentanoic acid {5-[3-(4-trifluoromethyl-benzylamino)-butyl]-pyridin-2-yl}-amide
2-[2-(3,5-Difluoro-phenyl)-acetylamino]-pentanoic acid [5-(3-benzylamino-butyl)-pyridin-2-yl]-amide
2-[2-(3,5-Difluoro-phenyl)-acetylamino]-pentanoic acid [5-(3-benzylamino-butyl)-pyridin-2-yl]-amide
2-[2-(3,5-Difluoro-phenyl)-acetylamino]-pentanoic acid (5-formyl-pyridin-2-yl)-amide
2-[2-(3,5-Difluoro-phenyl)-acetylamino]-pentanoic acid (5-[1-(4-methyl-piperidin-1-yl)-ethyl]-pyridin-2-yl}-amide
2-[2-(3,5-Difluoro-phenyl)-acetylamino]-pentanoic acid (5-vinyl-pyrazin-2-yl)-amide
2-[2-(3,5-Difluoro-phenyl)-acetylamino]-pentanoic acid {5-[1-(4-phenyl-piperidin-1-yl)-ethyl]-pyridin-2-yl}-amide
2-[2-(3,5-Difluoro-phenyl)-acetylamino]-pentanoic acid (5-formyl-pyrazin-2-yl)-amide
2-[2-(3-Trifluoromethyl-phenyl)-acetylamino]-pentanoic acid [5-(3-oxo-butyl)-pyridin-2-yl]-amide
2-[2-(3-Trifluoromethyl-phenyl)-acetylamino]-pentanoic acid [5-(1-methyl-3-oxo-butyl)-pyridin-2-yl]-amide
2-[2-(3-Trifluoromethoxy-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid (5-butylaminomethyl-pyrazin-2-yl)-amide
2-[2-(3,5-Difluoro-phenyl)-acetylamino]-pentanoic acid {5-[(3,3-dimethyl-butylamino)-methyl]-pyrazin-2-yl}-amide
2-[2-(3,5-Difluoro-phenyl)-acetylamino]-pentanoic acid {5-[(1-phenyl-propylamino)-methyl]-pyrazin-2-yl}-amide
2-[2-(3,5-Difluoro-phenyl)-acetylamino]-pentanoic acid {5-[(1-benzyl-pyrrolidin-3-ylamino)-methyl]-pyrazin-2-yl}-amide
2-[2-(3,5-Difluoro-phenyl)-acetylamino]-pentanoic acid [5-(1-hydroxy-ethyl)-pyrazin-2-yl]-amide
2-[2-(3,5-Difluoro-phenyl)-acetylamino]-pentanoic acid [5-(1-hydroxy-pentyl)-pyrazin-2-yl]-amide
2-[2-(3,5-Difluoro-phenyl)-acetylamino]-pentanoic acid [5-(4-methyl-piperazin-1-yl methyl)-pyrazin-2-yl]-amide
2-[2-(3,5-Difluoro-phenyl)-acetylamino]-pentanoic acid (5-hydroxymethyl-pyrazin-2-yl)-amide
2-[2-(3,5-Difluoro-phenyl)-acetylamino]-pentanoic acid [5-phenethylamino-methyl)-pyrazin-2-yl]-amide
2-[2-(3,5-Difluoro-phenyl)-acetylamino]-pentanoic acid [5-(isobutylamino-methyl)-pyrazin-2-yl]-amide
2-[2-(3,5-Difluoro-phenyl)-acetylamino]-pentanoic acid [5-(benzylamino-methyl)-pyrazin-2-yl]-amide
2-[2-(3,5-Difluoro-phenyl)-acetylamino]-pentanoic acid {5-[(3-methyl-butylamino)-methyl]-pyrazin-2-yl}-amide
2-[2-(3,5-Difluoro-phenyl)-acetylamino]-pentanoic acid {5-[(4-chloro-phenyl)-hydroxy-methyl]-pyrazin-2-yl}-amide
6-[2-(6,8-difluoro-1,2,3,4-tetrahydro-naphthalen-2-ylamino)-pentanoylamino]-nicotinic acid methyl ester
2-[2-(3,5-difluoro-phenyl)-acetylamino]-pentanoic acid [5-(1-hydroxy-1-methyl-allyl)-pyridin-2-yl]-amide
2-[2-(3,5-difluoro-phenyl)-acetylamino]-pentanoic acid {5-[1-(4-methyl-piperazin-1-yl)-ethyl]-pyridin-2-yl}-amide
2-[2-(3,5-difluoro-phenyl)-acetylamino]-pentanoic acid (5-{[methyl-(3-methyl-butyl)-amino]-methyl}-pyrazin-2-yl)-amide
2-[2-(3,5-Difluoro-phenyl)-acetylamino]-pentanoic acid {5-[(3-hydroxy-butylamino)-methyl]-pyrazin-2-yl}-amide
2-[2-(3,5-difluoro-phenyl)-acetylamino]-pentanoic acid {5-[(1-phenyl-ethylamino)-methyl]-pyrazin-2-yl}-amide
2-[2-(3,5-difluoro-phenyl)-acetylamino]-pentanoic acid (5-acetyl-pyrazin-2-yl)-amide
6-[2-(6,8-Difluoro-1,2,3,4-tetrahydro-naphthalen-2-ylamino)-pentanoylamino]-nicotinic acid methyl ester
2-(6,8-Difluoro-1,2,3,4-tetrahydro-naphthalen-2-ylamino)-pentanoic acid (5-bromo-pyrazin-2-yl)-amide
2-(6,8-Difluoro-1,2,3,4-tetrahydro-naphthalen-2-ylamino)-pentanoic acid pyrazin-2-ylamide
2-(6,8-Difluoro-1,2,3,4-tetrahydro-naphthalen-2-ylamino)-pentanoic acid (5-cyano-pyrazin-2-yl)-amide
2-(6,8-Difluoro-1,2,3,4-tetrahydro-naphthalen-2-ylamino)-pentanoic acid [5-(3-oxo-butyl)-pyrazin-2-yl]-amide
2-(5,7-Difluoro-1,2,3,4-tetrahydro-naphthalen-2-ylamino)-pentanoic acid {5-[3-(1-phenyl-ethylamino)-butyl]-pyridin-2-yl}-amide

Compounds of the Formula I of this invention, and their pharmaceutically acceptable salts, have useful pharmaceutical and medicinal properties. The compounds of Formula I, and their pharmaceutically acceptable salts inhibit the production of Aβ-peptide (thus, gamma-secretase activity) in mammals, including humans. Compounds of the Formula I, and their pharmaceutically acceptable salts, are therefore able to function as therapeutic agents in the treatment of the neurodegenerative and/or neurological disorders and diseases enumerated below, for example Alzheimer's disease, in an afflicted mammal, including a human.

The present invention also relates to a pharmaceutical composition for inhibiting Aβ-peptide production in a mammal, including a human, comprising an amount of a compound of the Formula I, or a pharmaceutically acceptable salt thereof, that is effective in inhibiting Aβ-production, and a pharmaceutically acceptable carrier.

The present invention also relates to a pharmaceutical composition for treating a disease or condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome in a mammal, including a human, comprising an amount of a compound of the Formula I, or a pharmaceutically acceptable salt thereof, that is effective in inhibiting Aβ-peptide production, and a pharmaceutically acceptable carrier.

The present invention also relates to a pharmaceutical composition for treating a disease or condition selected from the group consisting of Alzheimer's disease and Down's Syndrome in a mammal, including a human, comprising an amount of a compound of the Formula I, or a pharmaceutically acceptable salt thereof, that is effective in inhibiting Aβ-peptide production, and a pharmaceutically acceptable carrier.

The present invention also relates to a pharmaceutical composition for treating a disease or a condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome in a mammal, including a human, comprising an amount of a compound of the Formula I, or a pharmaceutically acceptable salt thereof, that is effective in treating such disease or condition, and a pharmaceutically acceptable carrier.

The present invention also relates to a pharmaceutical composition for treating a disease or a condition selected from the group consisting of Alzheimer's disease and Down's Syndrome in a mammal, including a human, comprising an amount of a compound of the Formula I, or a pharmaceutically acceptable salt thereof, that is effective in treating such disease or condition, and a pharmaceutically acceptable carrier.

The present invention also relates to a method of inhibiting Aβ-peptide production in a mammal, including a human, comprising administering to said mammal an amount of a compound of the Formula I, or a pharmaceutically acceptable salt thereof, that is effective in inhibiting Aβ-production.

The present invention also relates to a method of treating a disease or condition selected from Alzheimer's disease, hereditary cerebral hemorrhage with amyloidosis, cerebral amyloid angiopathy, a prion-mediated disease, inclusion body myositis, stroke, multiple sclerosis and Down's Syndrome in a mammal, including a human, comprising administering to said mammal an amount of a compound of the Formula I, or a pharmaceutically acceptable salt thereof, that is effective in inhibiting Aβ-production.

The present invention also relates to a method of treating a disease or condition selected from Alzheimer's disease and Down's Syndrome in a mammal, including a human, comprising administering to said mammal an amount of a compound of the Formula I, or a pharmaceutically acceptable salt thereof, that is effective in inhibiting Aβ-production.

The present invention also relates to a method of treating a disease or condition selected from Alzheimer's disease, hereditary cerebral hemorrhage with amyloidosis, cerebral amyloid angiopathy, a prion-mediated disease, inclusion body myositis, stroke, multiple sclerosis and Down's Syndrome in a mammal, including a human, comprising administering to said mammal an amount of a compound of the Formula I, or a pharmaceutically acceptable salt thereof, that is effective in treating such condition.

The present invention also relates to a method of treating a disease or condition selected from Alzheimer's disease and Down's Syndrome in a mammal, including a human, comprising administering to said mammal an amount of a compound of the Formula I, or a pharmaceutically acceptable salt thereof, that is effective in treating such condition.

The present invention also relates to a pharmaceutical composition for treating a disease or condition associated with Aβ-peptide production in a mammal, including a human, comprising (a) a compound of the Formula I, or a pharmaceutically acceptable salt thereof; (b) a memory enhancement agent, antidepressant, anxiolytic, antipsychotic agent, sleep disorder agent, anti-inflammatory agent, anti-oxidant agent, cholesterol modulating agent or anti-hypertensive agent; and (c) a pharmaceutically acceptable carrier; wherein the active agents “a” and “b” above are present in amounts that render the composition effective in treating such disease or condition.

The present invention also relates to a pharmaceutical composition for treating a disease or condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome, in a mammal, including a human, comprising (a) a compound of the Formula I, or a pharmaceutically acceptable salt thereof; (b) a memory enhancement agent, antidepressant, anxiolytic, antipsychotic agent, sleep disorder agent, anti-inflammatory agent, anti-oxidant agent, cholesterol modulating agent or anti-hypertensive agent; and (c) a pharmaceutically acceptable carrier; wherein the active agents “a” and “b” above are present in amounts that render the composition effective in treating such disease or condition.

The present invention also relates to a pharmaceutical composition for treating a disease or condition selected from the group consisting of Alzheimer's disease and Down's Syndrome, in a mammal, including a human, comprising (a) a compound of the Formula I, or a pharmaceutically acceptable salt thereof; (b) a memory enhancement agent, antidepressant, anxiolytic, antipsychotic agent, sleep disorder agent, anti-inflammatory agent, anti-oxidant agent, cholesterol modulating agent or anti-hypertensive agent; and (c) a pharmaceutically acceptable carrier; wherein the active agents “a” and “b” above are present in amounts that render the composition effective in treating such disease or condition.

The present invention also relates to a method of treating a disease or condition associated with Aβ-peptide production in a mammal, including a human, comprising administering to said mammal (a) a compound of the Formula I, or a pharmaceutically acceptable salt thereof; and (b) a memory enhancement agent, antidepressant, anxiolytic, antipsychotic agent, sleep disorder agent, anti-inflammatory agent, anti-oxidant agent, cholesterol modulating agent or anti-hypertensive agent; wherein the active agents “a” and “b” above are present in amounts that render the composition effective in treating such disease or condition.

The present invention also relates to a method of treating a disease or condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome, in a mammal, including a human, comprising administering to said mammal (a) a compound of the Formula I, or a pharmaceutically acceptable salt thereof; and (b) a memory enhancement agent, antidepressant, anxiolytic, antipsychotic agent, sleep disorder agent, anti-inflammatory agent, anti-oxidant agent, cholesterol modulating agent or anti-hypertensive agent; wherein the active agents “a” and “b” above are present in amounts that render the composition effective in treating such disease or condition.

The present invention also relates to a method of treating a disease or condition selected from the group consisting of Alzheimer's disease and Down's Syndrome, in a mammal, including a human, comprising administering to said mammal (a) a compound of the Formula I, or a pharmaceutically acceptable salt thereof; and (b) a memory enhancement agent, antidepressant, anxiolytic, antipsychotic agent, sleep disorder agent, anti-inflammatory agent, anti-oxidant agent, cholesterol modulating agent or anti-hypertensive agent; wherein the active agents “a” and “b” above are present in amounts that render the composition effective in treating such disease or condition.

Compounds in Formula I may be used alone or used as a combination with any other drug, including, but not limited to, any memory enhancement agent, such as donepezil, i.e., 2,3-dihydro-5,6-dimethyoxy-2[[1-(phenylmethyl)-4-piperidinyl]methyl]-1H-indene-1-one, e.g., Aricept™; antidepressant agent, such as sertraline, e.g., Zoloft™; anxiolytic, antipsychotic agent, such as ziprasidone, e.g., Geodon™; sleep disorder agent, anti-inflammatory agent such as celecoxib, i.e., 4-[5-4-methylphenyl)-3-(trifluoromethyl)-1H-pyrazol-1-yl]benzenesulfonamide, e.g., Celebrex™; valdecoxib, i.e., 4-(5-methyl-3-phenyl-4-isoxazolyl)benzenesulfonamide, e.g., Bextra™, and, anti-oxidant agent, cholesterol modulating agent (for example, an agent that lowers LDL or increases HDL), e.g., atorvastatin, such as Lipitor™, or anti-hypertension agent, and the like. Accordingly, this invention also provides a pharmaceutical composition for treatment of a mammal, including a human, in need thereof comprising an effective amount of a compound of Formula I and an effective amount of another drug, for example a memory enhancement agent, antidepressant agent, anxiolytic, antipsychotic agent, sleep disorder agent, anti-inflammatory agent, anti-oxidant agent, cholesterol modulating agent (for example, an agent that lowers LDL or increases HDL), or anti-hypertension agent, and a pharmaceutically acceptable carrier. This invention also provides a method for treating dementia, for example Alzheimer's disease, in a mammal, including in a human, comprising administering to the mammal an effective amount of a compound of Formula I and an effective amount of another drug, for example a memory enhancement agent, antidepressant agent, anxiolytic, antipsychotic agent, sleep disorder agent, anti-inflammatory agent, anti-oxidant agent, cholesterol modulating agent (for example, an agent that lowers LDL or increases HDL), or anti-hypertension agent.

Compounds of the Formula I, or any of the combinations described in the immediately preceding paragraph, may optionally be used in conjunction with a know P-glycoprotein inhibitor, such as verapamil.

References herein to diseases and conditions “associated with Aβ-peptide production” relate to diseases or conditions that are caused, at least in part, by Aβ-peptide and/or the production thereof. Thus, Aβ-peptide is a contributing factor, but not necessarily the only contributing factor, to “a disease or condition associated with Aβ-peptide production.”

As used herein, the term “treating” refers to reversing, alleviating or inhibiting the progress of a disease, disorder or condition, or one or more symptoms of such disease, disorder or condition, to which such term applies. As used herein, “treating” may also refer to decreasing the probability or incidence of the occurrence of a disease, disorder or condition in a mammal as compared to an untreated control population, or as compared to the same mammal prior to treatment. For example, as used herein, “treating” may refer to preventing a disease, disorder or condition, and may include delaying or preventing the onset of a disease, disorder or condition, or delaying or preventing the symptoms associated with a disease, disorder or condition. As used herein, “treating” may also refer to reducing the severity of a disease, disorder or condition or symptoms associated with such disease, disorder or condition prior to a mammal's affliction with the disease, disorder or condition. Such prevention or reduction of the severity of a disease, disorder or condition prior to affliction relates to the administration of the composition of the present invention, as described herein, to a subject that is not at the time of administration afflicted with the disease, disorder or condition. As used herein “treating” may also refer to preventing the recurrence of a disease, disorder or condition or of one or more symptoms associated with such disease, disorder or condition. The terms “treatment” and “therapeutically,” as used herein, refer to the act of treating, as “treating” is defined above.

### DETAILED DESCRIPTION OF THE INVENTION

Compounds of the Formula I, and their pharmaceutically acceptable salts, may be prepared as described in the following reaction Schemes and discussion. Unless otherwise indicated, as referred to in the reaction schemes and discussion that follow, R¹, R^{1a}, R^{1b}, R², R³, R⁴, R⁵, R⁶, R⁷, R⁸, R⁹, R¹⁰, R¹¹, R¹², X, Y, W. U, Z, m and n are as defined above.

The compounds of Formula I may have asymmetric carbon atoms and may therefore exist as racemic mixtures, diasteroisomers, or as individual optical isomers.

Separation of a mixture of isomers of compounds of Formula I into single isomers may be accomplished according to conventional methods known in the art.

The compounds of the Formula I may be prepared by the methods described below, together with synthetic methods known in the art of organic chemistry, or modifications and derivatisations that are familiar to those of ordinary skill in the art. Preferred methods include, but are not limited to, those described below.

The reactions described below are performed in solvents that are appropriate to the reagents and materials employed and that are suitable for use in the reactions described. In the description of the synthetic methods described below, it is also to be understood that all reaction conditions, whether actual or proposed, including choice of solvent, reaction temperature, reaction duration time, reaction pressure, and other reaction conditions (such as anhydrous conditions, under argon, under nitrogen, etc.), and work up procedures, are those conditions that are standard for that reaction, as would be readily recognized by one of skill in the art. Alternate methods may also be used.

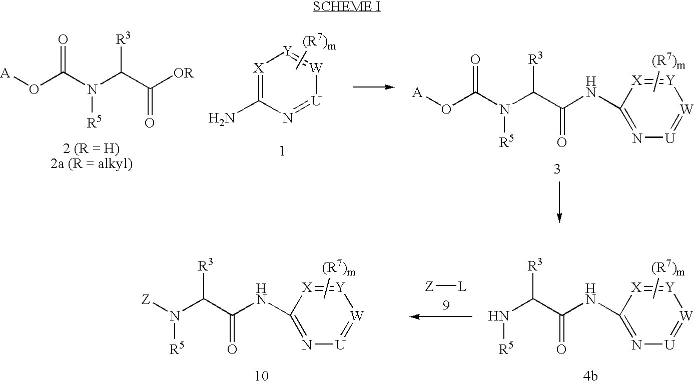

Scheme I refers to the preparation of compounds of the Formula I, 10. An amino-heteroaryl 1 is coupled with a nitrogen-protected amino acid 2. The nitrogen protecting group may be a carbamate-type such as butoxycarbonyl (“BOC”, A=tert-butyl) or benzyloxycarbonyl (“CBZ”, A=benzyl) that are prepared with either di-tert-butyl dicarbonate (Aldrich Chemical Company, Milwaukee Wis.) or with benzyl chloroformate (Aldrich) in the presence of either an inorganic or organic base (e.g., sodium carbonate or triethylamine) at 0 to 30° C. in an organic solvent (e.g., methylene chloride) or in a mixture of water and an organic solvent (e.g., ethyl acetate) (Scheme II) (see, Muller, Methoden Der Organischen Chemie. “Vierte Auglage —Synthesis von Peptiden I”—Houben Weyl—Georg-Thieme Verlag Stuttgart, 1974, Band XV/1).

Numerous reagents, well-known in the art, can be used to couple 1 and 2 to form 3 by standard peptide coupling methods known in art of organic chemistry (Scheme I). Activation of the carboxylic acid 2 with HATU (O-(7-azabenzotriazole-1-yl)-1,1,3,3,-tetramethyluronium hexafluorophosphate) or PyBOP (benzotriazole-1-yl)-oxy-tris-pyrrolidino-phosphonium hexafluorophosphate) or HBTU (O-benzotriazol-1-yl-N,N,N′,N′-tetramethyluronium hexafluorophosphate)/trialkylamine, or 1-hydroxybenzotriazole (HOBT)/1-(3-dimethylaminopropyl)-3-ethyl carbodiimide hydrochloride (EDC)/trialkylamine (e.g., NEt₃, NEt(iPr)₂) in an appropriate solvent such as methylene chloride, THF, DMF or a mixture of two solvents to have reagents mixed well to form a clear solution. Many of these peptide coupling agents or resins for solid phase synthesis such as Fmoc (Fluorenylmethylcarbonyl)-protected hydroxylamine bound to polystylene beads are common and well known in the literature. Deprotection of the Fmoc group may be achieved under standard conditions using 20% piperidine in DMF. References: O-benzotriazol-1-yl-N,N,N′N′-tetramethyluronium hexafluorophosphate (“HBTU”, Aldrich Chemical Company) and O-(7-azabenzotriazol-1-yt)-N,N,N′,N′-tetramethyluronium hexafluorophosphate (“HAT U”, Aldrich) (See, Fieser, Reagents for Organic Synthesis, 1986, Wiley Interscience, New York, Vol. 12, p. 44; Hruby, Biorganic Chemistry: Peptides and Proteins, 1998, Oxford University press, New York, pp. 27-64; Muller, Methoden Der Organischen Chemie, Vierte Auflage—Synthese von Peptiden II—Houben Weyl, George-Thieme Verlag Stuttgart, 1974, Band XVI2). When optically active reagents are employed, reaction conditions, such as temperature, time and the selection of the base, must be carefully controlled to avoid racemization. The protected amino group or carboxylic acid group may be prepared by methods well known in the literature for preparing amino acid protecting groups, as described in organic chemistry journals and textbooks such as “Protective Groups in Organic Synthesis,” by T. W. Green. Alternatively, the coupling may be performed by reacting I with the ester 2b in the presence of trialkylaluminum (e.g., AlMe₃) in an appropriate solvent, e.g., THF, toluene or a mixture of THF/toluene, in an open or sealed tube at a temperature between 0° C.-150° C. until complete conversion to the desired product (3 in Scheme I) is achieved; room temperature to 80° C. is preferred.

Intermediate 3 of Scheme I, is deprotected to afford aminoamide 4, either through treatment with strong acid in the case of t-butoxycarbonyl, or through hydrogenolysis in the case of carbobenzyloxycarbonyl. Specifically, t-BOC-3, on treatment with hydrochloric acid or trifluoroacetic acid in an organic solvent (e.g., dioxane, THF, or methylene chloride), at room temperature to 30° C. for about 1 hour to about 19 hours affords the corresponding salts of 4. Alternatively, CBZ-3 may be deprotected through catalytic hydrogenolysis in the presence of hydrogen (from about 1 to about 10 atmospheres), a heavy metal catalyst (e.g., palladium on carbon or palladium hydroxide on carbon, 1 to 10 percent catalyst loading, present at about 0.01 to about 0.50 times the of substrate), and a solvent (e.g., methanol, ethanol or ethyl acetate) at 20 to 50° C. for about 1 hour to about 19 hours.

The compound of Formula I, 10, in Scheme I may be prepared by reacting 4 with 9, where L is a leaving group (e.g., halide, mesylate, or triflate). The reaction is carried out at about 0° C. to about 30° C. in an inert solvent (e.g., methylene chloride, chloroform, THF, dichloroethane, ethyl acetate, acetonitrile or DMF) in the presence of an organic base (e.g., triethylamine, diisopropylethylamine or N-methylmorpholine) for about 1 minute to about 24 hours.

Alternatively, the compound of Formula I, 10, may be prepared according to the procedure of Scheme II, employing the general conditions described for Scheme I. In Scheme II, R may be alkyl or benzyl. The coupling of 9 and 11 in Scheme II may be performed at a temperature ranging from about 0° C. to about 30° C. in an organic solvent (e.g., methylene chloride, chloroform, dichloroethane ethyl acetate or DMF) in the presence of a base (e.g., triethylamine or diisopropylethylamine). When R is alkyl, either acidic or basic hydrolysis may be used to covert 12 to 13. If R is benzyl, catalytic hydrogenolysis may also be used to prepare 13.

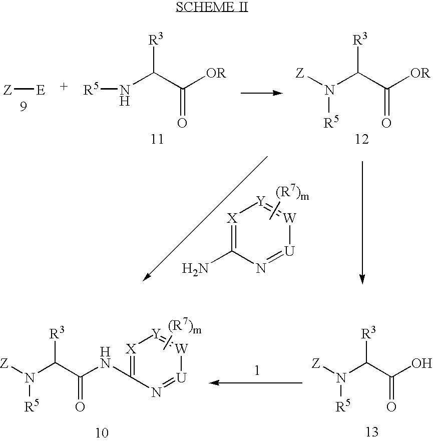

The above amide bond formation may be prepared by coupling the ester (12 in Scheme 11) with I in the presence of trialkylaluminum (e.g., AlMe₃) in an appropriate solvent, e.g., THF, toluene or a mixture of THF/toluene, in an open or sealed tube at temperature ranging from about 0° C. to about 110° C. until complete conversion to the desired product (10 in Scheme II) is achieved; room temperature to 80° C. is preferred.

Compounds in formula I wherein Z is R¹ may be prepared by method described in Scheme 1 or 11 using a reductive amination method in step of converting formula 4b to formula in Scheme I or in the step of converting formula 11 to formula 12 in Scheme II. For examples, reaction of intermediate 4b in Schem 1 or 11 in Scheme II with an appropriate aldehyde or ketone group to generate Z of R¹ of the corresponding formula 10 in Scheme I or formula 12 in Scheme II by a reductive amination method well established in the literature, such as in the presence of an appropriate reducing agents, preferably sodium cyanoborohydride or sodium triacetoxyborohydride in an appropriate solvent preferably methylene chloride, dichloroethane, DMF, THF, methanol, or ethanol or using conventional methods well established in the literature.

R⁷ of halo group may be generated by reacting the starting material, wherein R⁷ is H, with NBS, NCS or SO₂Cl₂, 12 in an appropriate solvent such as methylene chloride or chloroform. A halo group anywhere in the molecule may be replaced with another group by using methods well known in the art of organic chemistry, i.e., halogen-metal exchange, followed by quenching with an electrophile, or using typical Suzuki, Heck, Negishi, Sonogashira, Still coupling conditions employing a catalyst such as a palladium complex, e.g., tetrakis(triphenylphosphine)-palladium, Pd2(dba)₃/P(t-Bu)₃, palladium acetate, with sodium carbonate as a base in a suitable solvent such as THF, DME, ethanol, propionitrile, acetonitrile, toluene, and a boronic acid or tributyl-ylnyl-Tin/Pd₂(dba)₃/P(tol)₃ or related agents known in literature (References: Adam F. Liltke, Lothar Schwarz, and Gregory C. Fu*_{—}Am. Chem. Soc., 124 (22), 6343-6348, 2002; Littke, A. F.; Fu, G. C. Angew. Chem., Int Ed. Engl. 1998, 37, 3387-3388; Liftke, A. F.; Dai, C.; Fu, G. C. J. Am. Chem. Soc. 2000, 122, 4020-4028; Littke, A. F.; Fu, G. C. J. Org. Chem. 1999, 64, 10-11; Littke, A. F.; Fu, G. C. J. Am. Chem. Soc. 2001, 123, 6989-7000; Dai, C.; Fu, G. C. J. Am. Chem. Soc. 2001, 123, 2719-2724; Hundertmark, T.; Littke, A. F.; Buchwald, S. L.; Fu, G. C. Org. Lett. 2000, 2, 1729-1731. For pioneering studies by Koie of Pd/P(t-Bu)₃-catalyzed reactions, see: Nishiyama, M.; Yamamoto, T.; Koie, Y. Tetrahedron Lett. 1998, 39, 617-620; Yamamoto, T.; Nishiyama, M.; Koie, Y. Tetrahedron Left. 1998, 39, 2367-2370; Watanabe, M.; Nishiyama, M.; Koie, Y. Tetrahedron Lett. 1999, 40, 8837-8840. For early studies by others of Pd/P(t-Bu)₃-catalyzed reactions, see: Hartwig, J. F.; Kawatsura, M.; Hauck, S. I.; Shaughnessy, K. H.; Alcazar-Roman, L. M. J. Org. Chem. 1999, 64, 5575-5580; Mann, G.; Incarylto, C.; Rheingold, A. L.; Hartwig, J. F. J. Am. Chem. Soc. 1999, 121, 3224-3225; Kawatsura, M.; Hartwig, J. F. J. Am. Chem. Soc. 1999, 121, 1473-1478; Kim, J. S.; Sen, A. J. Mol. Catal. A 1999, 143, 197-201.) A halo group anywhere in the molecule may be replaced with another group by using Still cross coupling reactions A halo group anywhere in the molecule may be heated with an appropriate amine in an appropriate solvent (e.g., acetonitrile, propionitrile, DMSO, 1-methylpyrrolidin-2-one, DMF, toluene, ethanol), or in the presence of Pd(OAc)₂/(S)-BINAP/NaOt-Bu in an appropriate solvent (e.g., toluene) to provide the corresponding amino derivatives. The ester group may be converted to the corresponding amide using a similar method for amide bond formation, or using trimethylaluminum in an appropriate solvent or mixture of solvents, such as THF/toluene to yield the corresponding amide. The aldehyde or ketone group may undergo reductive amination to generate the corresponding amine derivatives using conventional methods well established in the literature. One or more protecting groups may be necessary during such reaction sequences or functional group changes in order to protect sensitive or reactive functional groups on any of the molecules involved in the reaction(s). Conventional protecting and deprotecting methods may be used according to methods well known in the art, such as those described in the literature or in textbooks, such as “Protective Groups in Organic Synthesis,” by T. W. Green.

The starting materials used in the procedures of the above Schemes, the syntheses of which are not described above, are either commercially available, known in the art or readily obtainable from known compounds using methods that will be apparent to those skilled in the art.

In the compounds described hereinabove, it is preferred that the carbon attached to the R³ is in the S-configuration. This embodiment is prepared from starting material in which the carbon atom attached to the R³ substituent is in the S configuration. The stereochemistry is derived from the utilization of the L-amino acid. Since during the synthesis, no substitution occurs at this asymmetric carbon, the sterochemical configuration is maintained throughout the synthesis to the final product.

The compounds of Formula I, and the intermediates shown in the above reaction schemes, may be isolated and purified by conventional procedures, such as recrystallization or chromatographic separation, such as on silica gel, either with an ethyl acetate/hexane elution gradient, a methylene chloride/methanol elution gradient, or a chloroform/methanol elution gradient. Alternatively, a reverse phase preparative HPLC or chiral HPLC separation technique may be used.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

Pharmaceutically acceptable salts of the compounds of Formula I may be prepared in a conventional manner by treating a solution or suspension of the corresponding free base or acid with one chemical equivalent of a pharmaceutically acceptable acid or base. Conventional concentration or crystallization techniques may be employed to isolate the salts. Suitable acids, include, but are not limited to, acetic, lactic, succinic, maleic, tartaric, citric, gluconic, ascorbic, benzoic, cinnamic, fumaric, sulfuric, phosphoric, hydrochloric, hydrobromic, hydroiodic, sulfamic, sulfonic acids such as methanesulfonic, benzene sulfonic, p-toluenesulfonic and related acids. Suitable bases include, but are not limited to, sodium, potassium and calcium.

A compound of the Formula I of the present invention may be administered to mammals via either the oral, parenteral (such as subcutaneous, intravenous, intramuscular, intrasternal and infusion techniques), rectal, intranasal, topical or transdermal (e.g., through the use of a patch) routes. In general, these compounds are most desirably administered in amounts effective for treating the above-identified diseases. Preferably, it is administered in doses ranging from about 0.1 mg to about 1000 mg per day, in single or divided doses (i.e., from 1 to 4 doses per day), although variations will necessarily occur depending upon the species, weight, age and condition of the subject being treated, as well as the particular route of administration chosen. However, a dosage level that is in the range of about 0.1 mg/kg to about 5 gm/kg body weight per day, preferably from about 0.1 mg/kg to about 100 mg/kg body weight per day, is most desirably employed. Nevertheless, variations may occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects, provided that such higher dosage levels are first divided into several small doses for administration throughout the day.

A compound of the Formula I of the present invention may be administered alone or in combination with pharmaceutically acceptable carriers or diluents by either of the routes previously indicated, and such administration may be carried out in single or multiple doses. Suitable pharmaceutical carriers include solid diluents or fillers, sterile aqueous media and various non-toxic organic solvents, etc. The pharmaceutical compositions formed by combining a compound of the Formula I, or a pharmaceutically acceptable salt thereof, with a pharmaceutically acceptable inert carrier, can then be readily administered in a variety of dosage forms such as tablets, capsules, lozenges, troches, hard candies, powders, sprays, creams, salves, suppositories, jellies, gels, pastes, lotions, ointments, aqueous suspensions, injectable solutions, elixirs, syrups, and the like. Moreover, oral pharmaceutical compositions may be suitably sweetened and/or flavored.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), methylcellulose, alginic acid and certain complex silicates, together with granulation binders such as polyvinylpyrrolidone, sucrose, gelatin and acacia. Additionally, lubricating agents such as magnesium stearate, sodium lauryl sulfate and talc are often useful for tabletting purposes. Solid compositions of a similar type may also be employed as fillers in gelatin capsules. Preferred materials in this connection include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions containing a compound of the Formula I of the present invention in either sesame or peanut oil or in aqueous propylene glycol may be employed. The aqueous solutions should be suitably buffered (preferably pH greater than 8) if necessary and the liquid diluent first rendered isotonic with sufficient saline or glucose. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

The compounds of Formula I of the present invention are useful in inhibiting Aβ-peptide production (thus, gamma-secretase activity) in mammals, and therefore they are able to function as therapeutic agents in the treatment of the aforementioned disorders and diseases in an afflicted mammal.

The ability of compounds of the Formula I of this invention, and their pharmaceutically acceptable salts, to inhibit Aβ-peptide production (thus, gamma-secretase activity) may be determined using biological assays known to those of ordinary skill in the art, for example the assays described below.

The activity of compounds of the Formula I of the present invention in inhibiting gamma-secretase activity is determinable in a solubilized membrane preparation generally according to the description provided in McLendon et al. Cell-free assays for γ-secretase activity, The FASEB Journal (Vol. 14, December 2000, pp. 2383-2386). Compounds of the present invention were determined to have an IC₅₀ activity for inhibiting gamma-secretase activity of less than about 100 micromolar.

The following Examples illustrate the present invention. It is to be understood, however, that the invention, as fully described herein and as recited in the claims, is not intended to be limited by the details of the following Examples.

### EXPERIMENTAL PROCEDURES

### Example 1

### 2-[2-(5-Bromo-pyridin-3-yl)-acetylamino]-N-pyrazin-2-yl-propionamide

A mixture of 5-bromo-3-pyridyl acetic acid (64.7 mg, 0.30 mmol), 2-Amino-N-pyrazin-2-yl-propionamide hydrochloride 55 mg, 0.27 mmol), HOBT (44 mg, 0.33 mmol), EDC. HCl (70.2 mg, 0.41 mmol), and triethylamine (0.11 ml, 1.4 mmol) in methylene chloride was stirred at room temperature over night. The mixture was quenched with water and extracted with methylene chloride. The organic layer was washed with dil HCl to pH 6.5, separated, dried and concentrated to give 79 mg of the title compound as a glass form. The glass form was purified by preparative HPLC to provide the title compound as a white solid. ¹H NMR (DMSO-d₆) d 10.9(s,1H), 8.56(s,1H), 8.43(d,1H), 8.40(s,1H), 8.39(d,2H), 8.35(s,1H), 7.94(s,1H), 4.51(m,1H), 3.56(s,2H), 1.32(d,3H) ppm. M+1=364.2.

The following examples were prepared by the method analogous to that described in Example 1 by reacting an appropriate acid, an amine, HOBT and EDC HCl.

### Example 2

### 2-[2-(5-Bromo-pyridin-3-yl)-acetylamino]-pentanoic Acid pyrazin-2-ylamide

The title compound was prepared by coupling 5-bromo-3-pyridyl acetic acid, 2-amino-pentanoic acid pyrazin-2-ylamide hydrochloride using HOBT/EDC.HCl coupling agent. LC-MS M+1=392.3.

### Example 3

### 2-(2-Hydroxy-3-methyl-butyrylamino)-pentanoic Acid pyrazin-2-ylamide

The title compound was prepared by coupling (S)-2-hydroxy-3-mehtylbutyric acid, 2-Amino-pentanoic acid pyrazin-2-ylamide hydrochloride using HOBT/EDC.HCl coupling agent. LC-MS M+1=295.4.

### Example 4

### 2-[2-(3,5-Difluoro-phenyl)-acetylamino]-N-pyrazin-2-yl-butyramide

The title compound was prepared by coupling 3,5-difluoro-acetic acid, 2-amino-N-pyrazin-2-ylbutyramide hydrochloride using HOBT/EDC.HCl coupling agent. LC-MS M+1=335.3.

### Example 5

### 2-[2-(3,5-Difluoro-phenyl)-acetylamino]-pentanoic Acid pyrazin-2-ylamide

The title compound was prepared by coupling 3,5-difluoro-acetic acid, 2-amino-pentanoic acid pyrazin-2-ylamide hydrochloride using HOBT/EDC.HCl coupling agent. LC-MS M+1=349.3.

### Example 6

### 2-(2-Hydroxy-3,3-dimethyl-butyrylamino)-pentanoic Acid pyrazin-2-ylamide

The title compound was prepared by coupling S-(−)-2-hydroxy-3,3-dimethyl-butyric acid, 2-amino-pentanoic acid pyrazin-2-ylamide hydrochloride using HOBT/EDC.HCl coupling agent. LC-MS M+1=309.3.

### Example 7

### 2-[2-(3-phenoxy-phenyl)-acetylamino]-N-pyrazin-2-yl-propionamide

The title compound was prepared by coupling 3-phenoxyphenyl acetic acid, 2-amino-pyrazin-2-yl-propionamide hydrochloride using HOBT/EDC.HCl coupling agent. LC-MS M+1=377.3.

### Example 8

### 2-[2-(3,5-Difluoro-phenyl)-2-(S)-hydroxy-acetylamino]-pentanoic Acid pyrazin-2-ylamide

The title compound was prepared by coupling racemic 3,5-difluoro-mandelic acid, 2-amino-pentanoic acid pyrazin-2-ylamide hydrochloride using HOBT/EDC.HCl coupling agent. 2-(S)-hydroxy-isomer of the title compound was separated. LC-MS M+1=365.3.

### Example 9

### 2-[2-(3,5-Difluoro-phenyl)-2-(R)-hydroxy-acetylamino]-pentanoic Acid pyrazin-2-ylamide

The title compound was prepared by coupling 3,5-difluoro-mandelic acid, 2-amino-pentanoic acid pyrazin-2-ylamide hydrochloride using HOBT/EDC.HCl coupling agent. 2-(R)-hydroxy-isomer of the title compound was separated. LC-MS M+1=365.3.

### Example 10

### 2-[2-(3,5-Difluoro-phenyl)-acetylamino]-N-pyridin-2-yl-butyramide

A mixture of 2-amino-pyridine (208 mg, 0.369 mmol), 2M trimethylaluminum in toluene (0.37 ml, 0.74 mmol), and (2S)-2-{[3,5-difluorophenyl)acetyl]amino}butanoic acid methyl ester (100 mg, 0.37 mmol) in 3 ml of THF was heated at 90° C. overnight. The mixture was quenched with dilute HCl and extracted with chloroform. The organic layer was separated, dried over anhydrous sodium sulfate, filtered, and concentrated to dryness. The residue was purified by Shimadzu HPLC to give the title compound as a white solid. LC-MS RT=1.9 min, M+1=334.5.

The following examples were prepared by the method analogous to that described in Example 10 starting with an appropriate ester, an amine and trimethylaluminum.

### Example 11

### N-(5-Bromo-pyridin-2-yl)-2-[2-(3,5-difluoro-phenyl)-acetylamino]-butyramide

The title compound was prepared by reacting (2S)-2-{[3,5-difluorophenyl)acetyl]amino}butanoic acid methyl ester, 2-amino-5-bromo-pyridine and trimethylaluminum. LC-MS M+1=414.4.

### Example 12

### 2-[2-(3,5-Difluoro-phenyl)-acetylamino]-N-(5-iodo-pyridin-2-yl)-butyramide

The title compound was prepared by reacting (2S)-2-{[3,5-difluorophenyl)acetyl]amino}butanoic acid methyl ester, 2-amino-5-iodo-pyridine and trimethylaluminum. LC-MS M+1=460.4.

### Example 13

### 2-[2-(3-phenoxy-phenyl)-acetylamino]-pentanoic Acid pyrazin-2-ylamide

The title compound was prepared by reacting (2S)-2-{[3-phenoxyphenyl)acetyl]amino}pentanoic acid methyl ester, 2-amino-pyrazine and trimethylaluminum. LC-MS M+1=405.6.

### Example 14

### 2-[2-(3-phenoxy-phenyl)-acetylamino]-pentanoic Acid pyrimidin-2-ylamide

The title compound was prepared by reacting (2S)-2-{[3-phenoxyphenyl)acetyl]amino}pentanoic acid methyl ester, 2-amino-pyrimidine and trimethylaluminum. LC-MS M+1=405.6.

### Example 15

### 2-[2-(3-phenoxy-phenyl)-acetylamino]-pentanoic Acid (6-chloro-pyridazin-3-yl)-amide

The title compound was prepared by reacting (2S)-2-{[3-phenoxyphenyl)acetyl]amino}pentanoic acid methyl ester, 3-amino-6-chloro-pyridazine and trimethylaluminum. LC-MS M+1=439.6.

### Example 16

### 2-[2-(3-phenoxy-phenyl)-acetylamino]-pentanoic Acid pyrimidin-4-ylamide

The title compound was prepared by reacting (2S)-2-{[3-phenoxyphenyl)acetyl]amino}pentanoic acid methyl ester, 4-amino-pyrimidine and trimethylaluminum. LC-MS M+1=405.6.

### Example 17

### 2-[2-(3-phenoxy-phenyl)-acetylamino]-pentanoic Acid (4-methyl-pyrimidin-2-yl)-amide

The title compound was prepared by reacting (2S)-2-{[3-phenoxyphenyl)acetyl]amino}pentanoic acid methyl ester, 2-amino-4-methyl-pyrimidine and trimethylaluminum. LC-MS M+1=419.6.

### Example 18

### 2-[2-(3-phenoxy-phenyl)-acetylamino]-pentanoic Acid (4-chloro-6-methyl-pyrimidin-2-yl)-amide

The title compound was prepared by reacting (2S)-2-{[3-phenoxyphenyl)acetyl]amino}pentanoic acid methyl ester, 2-amino-4-methyl-6-chloro-pyrimidine and trimethylaluminum. LC-MS M+1=453.4.

### Example 19

### 2-[2-(3-phenoxy-phenyl)-acetylamino]-pentanoic Acid (6-chloro-pyrazin-2-yl)-amide

The title compound was prepared by reacting (2S)-2-{[3-phenoxyphenyl)acetyl]amino}pentanoic acid methyl ester, 2-amino-6-chloro-6-chloro-pyrazine and trimethylaluminum. LC-MS M+1=439.4.

### Example 20

### 2-[2-(3,5-Difluoro-phenyl)-acetylamino]-pentanoic Acid (6-chloro-pyridazin-3-yl)-amide

The title compound was prepared by reacting methyl (2S)-2-{[(3,5-difluoro-phenyl)-acetyl]amino]-pentanoate, 2-amino-6-chloro-pyridazine and trimethylaluminum. LC-MS M+1=383, RT=2.2 min.

### Example 21

### 2-[2-(3,5-Difluoro-phenyl)-acetylamino]-pentanoic Acid pyrimidin-4-ylamide

The title compound was prepared by reacting methyl (2S)-2-{[(3,5-difluoro-phenyl)-acetyl]amino]-pentanoate, 4-amino-pyrimidine and trimethylaluminum. LC-MS M+1=349.2.

### Example 22

### 2-[2-(3,5-Difluoro-phenyl)-acetylamino]-pentanoic Acid (5-trifluoromethyl-pyridin-2-yl)-amide

The title compound was prepared by reacting methyl (2S)-2-{[(3,5-difluoro-phenyl)-acetyl]amino]-pentanoate, 4-amino-5-trifluormethyl-pyridine and trimethylaluminum. LC-MS M+1=416.1.

### Example 23

### 6-[2-[2-(3,5-Difluoro-phenyl)-acetylamino]-pentanoylamino]-nicotinamide

The title compound was prepared by reacting methyl (2S)-2-{[(3,5-difluoro-phenyl)-acetyl]amino]-pentanoate, 6-amino-nicotinamide and trimethylaluminum. LC-MS M+1=391 RT=1.9 min.

### Example 24

### 2-[2-(3-phenoxy-phenyl)-acetylamino]-pentanoic Acid [6-(1-ethyl-propylamino)-pyrazin-2-yl]-amide

A mixture of 2-[2-(3-phenoxy-phenyl)-acetylamino]-pentanoic acid [6-chloro-pyrazin-2-yl]-amide (68 mg, 0.156 mmol) and N-ethyl-N-propyl amine (0.3 ml) in DMSO (0.5 mL) was heated at 115° C. overnight. The mixture was quenched with water and extracted with ethyl acetate. The organic layer was separated, dried, concentrated and purified by preparative HPLC to give the title compound as a brown solid. LC-MS M+1=490, RT=3.0 min.

The following examples were prepared by the method analogous to that described in the example 24.

### Example 25

### 2-[2-(3-phenoxy-phenyl)-acetylamino]-pentanoic Acid (6-ethylamino-pyrazin-2-yl)-amide

The title compound was prepared by heating 2-[2-(3-phenoxy-phenyl)-acetylamino]-pentanoic acid [6-chloro-pyrazin-2-yl]-amide and N-ethylamine. LC-MS M+1=448 at RT=2.6 min.

### Example 26

### 2-[2-(3-phenoxy-phenyl)-acetylamino]-pentanoic Acid [6-(butyl-methyl-amino)-pyrazin-2-yl]-amide

The title compound was prepared by heating 2-[2-(3-phenoxy-phenyl)-acetylamino]-pentanoic acid [6-chloro-pyrazin-2-yl]-amide and N-methyl-N-butylamine. LC-MS M+1=490 at RT=3.1 min.

### Example 27

### 2-[2-(3,5-Difluoro-phenyl)-acetylamino]-pentanoic Acid [5-(4-chloro-phenoxy)-pyrimidin-2-yl]-amide

A mixture of 5-(4-Chloro-phenoxy)-pyrimidin-2-ylamine (45.08 mg, 0.2 mmol) and 2M trimethylaluminum in toluene (0.1 ml, 0.2 mmol) in 1 ml of methylene chloride was stirred at room temperature for 1 hr. A solution of 2-[2-(3,5-Difluoro-phenyl)-acetylamino]-pentanoic acid methyl ester (57 mg, 0.2 mmol) in 1.5 ml of methylene chloride was added to the reaction mixture and the resulting mixture was heated at 41° C. overnight. The mixture was quenched with dilute HCl and extracted with methylene chloride. The organic layer was separated, dried over anhydrous sodium sulfate, filtered, and concentrated to dryness. The residue was purified by Shimadzu HPLC to give the title compound as a white solid. LC-MS, RT=2.6 min, M+1=475.2.

The following examples 28-45 were prepared by the method analogous to that described in the 27 starting form an appropriate substituted pyrimidine-2-ylamine (0.2 mmol) and 2 M trimehtylaluminum in toluene in methylene chloride, followed by addition of 2-[2-(3,5-difluoro-phenyl)-acetylamino]-pentanoic acid methyl ester (0.2 mmol) to give the desired title compound after HPLC purification.

### Example 28

### 2-[2-(3,5-Difluoro-phenyl)-acetylamino]-pentanoic Acid (5-o-tolyloxy-pyrimidin-2-yl)-amide,

white solid, LC-MS, RT=2.6 min, M+1=455.3

### Example 29

### 2-[2-(3,5-Difluoro-phenyl)-acetylamino]-pentanoic Acid (5-m-tolyloxy-pyrimidin-2-yl)-amide,

white solid, LC-MS, RT=2.6 min, M+1=455.3

### Example 30

### 2-[2-(3,5-Difluoro-phenyl)-acetylamino]-pentanoic acid [5-(3-tert-butyl-phenoxy)-pyrimidin-2-yl-amide,

white solid, LC-MS, RT=2.9 min, M+1=497.4.

### Example 31

### 2-[2-(3,5-Difluoro-phenyl)-acetylamino]-pentanoic Acid (5-pentyloxy-pyrimidin-2-yl)-amide,

white solid, LC-MS, RT=2.6 min, M+1=435.3.

### Example 32

### 2-[2-(3,5-Difluoro-phenyl)-acetylamino]-pentanoic Acid [5-(3-trifluoromethyl-phenoxy)-pyrimidin-2-yl]-amide,

white solid, LC-MS RT=2.8 min, M+1=509.2.

### Example 33

### 2-[2-(3,5-Difluoro-phenyl)-acetylamino]-pentanoic Acid [5-(naphthalen-2-yloxy)-pyrimidin-2-yl]-amide,

white solid, LC-MS RT=2.8 min, M+1=491.3.

### Example 34

### 2-[2-(3,5-Difluoro-phenyl)-acetylamino]-pentanoic Acid [5-(4-methoxy-phenoxy)-pyrimidin-2-yl]-amide,

white solid, LC-MS RT=2.5 min, M+1=471.5.

### Example 35

### 2-[2-(3,5-Difluoro-phenyl)-acetylamino]-pentanoic Acid (5-p-tolyloxy-pyrimidin-2-yl)-amide,

white solid, LC-MS RT=2.7 min, M+1=455.3.

### Example 36

### 2-[2-(3,5-Difluoro-phenyl)-acetylamino]-pentanoic Acid (5-methyl-pyrimidin-2-yl)-amide,

white solid, LC-MS RT=1.8 min, M+1=363.2.

### Example 37

### 2-[2-(3,5-Difluoro-phenyl)-acetylamino]-pentanoic Acid (5-bromo-pyrimidin-2-yl)-amide,

white solid, LC-MS RT=2.1 min, M+1=429.1.

### Example 38

### 2-[2-(3,5-Difluoro-phenyl)-acetylamino]-pentanoic Acid (5-chloro-pyrimidin-2-yl)-amide,

white solid, LC-MS RT=2.1 min, M+1=286.1.

### Example 39

### 3-(6-2-[2-(3,5-Difluoro-phenyl)-acetylamino]-pentanoylamino]-pyridin-3-yl)-but-2-enoic Acid methyl ester,

white glass foam, LC-MS RT=2.6 min, M+1=446.5.

### Example 40

### 2-[2-(3,5-Difluoro-phenyl)-acetylamino]-pentanoic Acid (5-benzyl-pyridin-2-yl)-amide,

white solid, LC-MS RT=2.7 min, M+1=438.4

### Example 41

### 4-(6-[2-[2-(3,5-Difluoro-phenyl)-acetylamino]-pentanoylamino]-pyridin-3-yl)-piperazine-1-carboxylic acid tert-butyl ester,

brown solid, LC-MS RT=2.6 min, M+1=532.5.

### Example 42

### 2-[2-(3,5-Difluoro-phenyl)-acetylamino]-pentanoic Acid (5-bromo-pyridin-2-yl)-amide,

white solid, LC-MS, RT=2.6 min, M+1=426.1.

### Example 43

### 3-(6-{2-[2-(3,5-Difluoro-phenyl)-acetylamino]-pentanoylamino}-pyridin-3-yl)-acrylic Acid methyl ester,

white solid, LC-MS, RT=2.49 min, M+1=432.2.

### Example 44

### 3-(6-[2-[2-(3,5-Difluoro-phenyl)-acetylamino]-pentanoylamino]-pyridin-3-yl)-butyric Acid methyl ester

A mixture of 3-(6-{2-[2-(3,5-Difluoro-phenyl)-acetylamino]-pentanoylamino}-pyridin-3-yl)-but-2-enoic acid methyl ester (44 mg, 0.010 mmol) and 10 mg of PtO2 in ethyl acetate was hydrogenated at 45 psi for 6 hr. The reaction mixture was filtered through celite. The filtrate was concentrated to dryness to give 33.6 mg of the title compound as a glass solid. LC-MS RT=2.41 min, M+1=448.2.

### Example 45

### 6-[2-[2-(3,5-Difluoro-phenyl)-acetylamino]-pentanoylamino]-nicotinic Acid ethyl ester

A mixture of (2S)-2-{[(3,5-difluoro-phenyl)-acetyl]amino]-pentanoate (58 mg, 0.2 mmol), 6-amino-nicotinic acid ethyl ester (34.8 mg, 0.2 mmol), HOBT (33 mg, 0.24 mmol), EDC.HCl (59 mg, 0.31 mmol), and triethylamine (82.5 mg, 0.8 mmol) in methylene chloride was stirred at room temperature over weekend. The mixture was quenched with water and extracted with methylene chloride. The organic layer was washed with dil HCl to pH 6.5, separated, dried and concentrated. The residue was purified by shimadzu HPLC to provide the title-compound, LC-MS, M+1=420.5 at RT=2.5 min.

### Example 46

### 2-[2-(3,5-Difluoro-phenyl)-acetylamino]-pentanoic Acid (5-cyano-pyridin-2-yl)-amide

6-Amino-nicotinonitrile (1.0 g, 8.4 mmol) and trimethylaluminum (4.2 mL of 2.0 M solution, 8.4 mmol) were stirred in tetrahydrofuran at room temperature for 2 hours. 2-[2-(3,5-difluoro-phenyl)-acetylamino]-pentanoic acid (1.14 g, 4.2 mmol) was then added to the solution and stirred at 50° C. overnight. The mixture was quenched with Rochelle Salts in water and extracted with ethyl acetate. The organic layer was concentrated and purified by biotage column to give title compound as a yellow solid. APCI-MS [M+1]=373.2 The following examples 47-53 were prepared by the method analogous to that described in Example 46.

### Example 47

### 2-[2-(3,5-Difluoro-phenyl)-acetylamino]-pentanoic Acid (6-chloro-pyridazin-3-yl)-amide

was prepared starting from 2-[2-(3,5-difluoro-phenyl)-acetylamino]-pentanoic acid methyl ester, 6-chloro-pyridazin-3-ylamine and trimethylaluminum to obtain a yellow solid, APCI-MS [M+1]=383.1.

### Example 48

### 2-[2-(3,5-Difluoro-phenyl)-acetylamino]-pentanoic Acid [5-(1-hydroxy-1-methyl-ethyl)-pyridin-2-yl]-amide

was prepared starting from 2-[2-(3,5-difluoro-phenyl)-acetylamino]-pentanoic acid methyl ester, 2-(6-amino-pyridin-3-yl)-propan-2-ol and trimethylaluminum to obtain a yellow glass form, APCI-MS [M+1]=406.2.

### Example 49

### 2-[2-(3,5-Difluoro-phenyl)-acetylamino]-pentanoic Acid (5-bromo-pyrazin-2-yl)-amide

was prepared starting from 2-[2-(3,5-difluoro-phenyl)-acetylamino]-pentanoic acid methyl ester, 5-bromo-pyrazin-2-ylamine and trimethylaluminum to give a white solid, APCI-MS [M+1]=429.1.

### Example 50

### 2-[2-(3,5-Difluoro-phenyl)-acetylamino]-pentanoic Acid [5-(1-hydroxy-1-methyl-pentyl)-pyridin-2-yl]-amide

was prepared by reacting 2-[2-(3,5-difluoro-phenyl)-acetylamino]-pentanoic acid methyl ester, 2-(6-amino-pyridin-3-yl)-hexan-2-ol and trimethylaluminum. The title compound was obtained as a white glass form. APCI-MS [M+1]=448.3.

### Example 51

### 2-[2-(3,5-Difluoro-phenyl)-acetylamino]-pentanoic Acid [5-(1-butyl-ylnyl)-pyridin-2-yl]-amide

was prepared by reacting 2-[2-(3,5-difluoro-phenyl)-acetylamino]-pentanoic acid methyl ester, 2-(6-amino-pyridin-3-yl)-hexan-2-ol and trimethylaluminum. The title compound was obtained as a yellow oil. APCI-MS [M+1]=430.3.

### Example 52

### 2-[2-(3,5-Difluoro-phenyl)-acetylamino]-pentanoic Acid (5-acetyl-pyridin-2-yl)-amide

was prepared by reacting 2-[2-(3,5-difluoro-phenyl)-acetylamino]-pentanoic acid methyl ester, 1-(6-amino-pyridin-3-yl)-ethanone and trimethylaluminum. The title compound was obtained as a white solid. APCI-MS [M+1]=390.1.

### Example 53

### 6-[2-[2-(3,5-Difluoro-phenyl)-acetylamino]-pentanoylamino]-nicotinic Acid Methyl Ester

was prepared by reacting 2-[2-(3,5-difluoro-phenyl)-acetylamino]-pentanoic acid methyl ester, 6-amino-nicotinic acid methyl ester and trimethylaluminum. The title compound was obtained as a clear oil. APCI-MS [M+1]=406.1.

### Example 54

### 2-[2-(3,5-Difluoro-phenyl)-acetylamino]-pentanoic Acid {5-[methyl-(3-methyl-butyl)-amino]-pyrazin-2-yl}-amide

2-[2-(3,5-difluoro-phenyl)-acetylamino]-pentanoic acid (5-bromo-pyrazin-2-yl)-amide (150 mg, 0.35 mmol) and methyl-(3-methyl-butyl)-amine (71 mg, 0.70 mmol) were stirred in dimethyl sulfoxide at 100° C. for 3 hours. The mixture was quenched with water and extracted with ethyl acetate. The organic layer was concentrated and purified by preparative HPLC to give the title compound as a yellow solid. APCI-MS [M+1]=448.3 The following examples 55-75 were prepared by the method analogous to that described in Example 54.

### Example 55

### 2-[2-(3,5-Difluoro-phenyl)-acetylamino]-pentanoic Acid [5-(butyl-methyl-amino)-pyrazin-2-yl]-amide

The title compound was prepared by reacting 2-[2-(3,5-difluoro-phenyl)-acetylamino]-pentanoic acid (5-bromo-pyrazin-2-yl)-amide and butyl-methyl-amine. The title compound was obtained as a light beige solid. APCI-MS [M+1]=434.3.

### Example 56

### 2-[2-(3,5-Difluoro-phenyl)-acetylamino]-pentanoic Acid [5-(butyl-ethyl-amino)-pyrazin-2-yl]-amide

The title compound was prepared by reacting 2-[2-(3,5-difluoro-phenyl)-acetylamino]-pentanoic acid (5-bromo-pyrazin-2-yl)-amide and butyl-ethyl-amine. The title compound was obtained as a yellow oil. APCI-MS [M+1]=448.3.

### Example 57

### 2-[2-(3,5-Difluoro-phenyl)-acetylamino]-pentanoic Acid [5-(cyclopropylmethyl-propyl-amino)-pyrazin-2-yl]-amide

The title compound was prepared by reacting 2-[2-(3,5-difluoro-phenyl)-acetylamino]-pentanoic acid (5-bromo-pyrazin-2-yl)-amide and cyclopropyl-propyl-amine. The title compound was obtained as a white solid. APCI-MS [M+1]=460.3.

### Example 58

### 2-[2-(3,5-Difluoro-phenyl)-acetylamino]-pentanoic Acid [5-(hexyl-methyl-amino)-pyrazin-2-yl]-amide

The title compound was prepared by reacting 2-[2-(3,5-difluoro-phenyl)-acetylamino]-pentanoic acid (5-bromo-pyrazin-2-yl)-amide and hexyl-methyl-amine. The title compound was obtained as a white solid. APCI-MS [M+1]=462.3.

### Example 59

### 2-[2-(3,5-Difluoro-phenyl)-acetylamino]-pentanoic Acid {6-[methyl-(3-methyl-butyl)-amino]-pyridazin-3-yl}-amide

The title compound was prepared by reacting 2-[2-(3,5-difluoro-phenyl)-acetylamino]-pentanoic acid (6-chloro-pyridazin-3-yl)-amide and methyl-(3-methyl-butyl)-amine. The title compound was obtained as a yellow oil. APCI-MS [M+1]=448.3.

### Example 60

### 2-[2-(3,5-Difluoro-phenyl)-acetylamino]-pentanoic Acid [6-(butyl-methyl-amino)-pyridazin-3-yl]-amide

The title compound was prepared by reacting 2-[2-(3,5-difluoro-phenyl)-acetylamino]-pentanoic acid (6-chloro-pyridazin-3-yl)-amide and butyl-methyl-amine. The title compound was obtained as a yellow oil. APCI-MS [M+1]=434.3.

### Example 61

### 2-[2-(3,5-Difluoro-phenyl)-acetylamino]-pentanoic Acid [6-(1-ethyl-Propylamino)-pyridazin-3-yl]-amide

The title compound was prepared by reacting 2-[2-(3,5-difluoro-phenyl)-acetylamino]-pentanoic acid (6-chloro-pyridazin-3-yl)-amide and 1-ethyl-propylamine. The title compound was obtained as a white solid. APCI-MS [M+1]=434.3.

### Example 62

### 2-[2-(3,5-Difluoro-phenyl)-acetylamino]-pentanoic Acid [6-(butyl-ethyl-amino)-pyridazin-3-yl]-amide

The title compound was prepared by reacting 2-[2-(3,5-difluoro-phenyl)-acetylamino]-pentanoic acid (6-chloro-pyridazin-3-yl)-amide and butyl-ethyl-amine. The title compound was obtained as a yellow oil. APCI-MS [M+1]=448.3.

### Example 63

### 2-[2-(3,5-Difluoro-phenyl)-acetylamino]-pentanoic Acid [6-(hexyl-methyl-amino)-pyridazin-3-yl-amide

The title compound was prepared by reacting 2-[2-(3,5-difluoro-phenyl)-acetylamino]-pentanoic acid (6-chloro-pyridazin-3-yl)-amide and hexyl-methyl-amine. The title compound was obtained as a yellow oil. APCI-MS [M+1]=462.3.

### Example 64

### 2-[2-(3,5-Difluoro-phenyl)-acetylamino]-pentanoic Acid (5-morpholin-4-yl-pyrazin-2-yl)-amide

The title compound was prepared by reacting 2-[2-(3,5-difluoro-phenyl)-acetylamino]-pentanoic acid (5-bromo-pyrazin-2-yl)-amide and morpholine. The title compound was obtained as a white solid. APCI-MS [M+1]=434.2.

### Example 65

### 2-[2-(3,5-Difluoro-phenyl)-acetylamino]-pentanoic Acid (5-phenethylamino-pyrazin-2-yl)-amide

The title compound was prepared by reacting 2-[2-(3,5-difluoro-phenyl)-acetylamino]-pentanoic acid (5-bromo-pyrazin-2-yl)-amide and phenethylamine. The title compound was obtained as a yellow solid. APCI-MS [M+1]=468.2.

### Example 66

### 2-[2-(3,5-Difluoro-phenyl)-acetylamino]-pentanoic Acid [5-(benzyl-methyl-amino)-pyrazin-2-yl]-amide

The title compound was prepared by reacting 2-[2-(3,5-difluoro-phenyl)-acetylamino]-pentanoic acid (5-bromo-pyrazin-2-yl)-amide and benzyl-methyl-amine. The title compound was obtained as a white solid. APCI-MS [M+1]=468.2.

### Example 67

### 2-[2-(3,5-Difluoro-phenyl)-acetylamino]-pentanoic Acid (5-dibenzylamino-pyrazin-2-yl)-amide

The title compound was prepared by reacting 2-[2-(3,5-difluoro-phenyl)-acetylamino]-pentanoic acid (5-bromo-pyrazin-2-yl)-amide and dibenzyl-amine. The title compound was obtained as a white solid. APCI-MS [M+1]=544.3.

### Example 68

### 2-[2-(3,5-Difluoro-phenyl)-acetylamino]-pentanoic Acid [5-(1-methoxymethyl-Propylamino)-pyrazin-2-yl]-amide

The title compound was prepared by reacting 2-[2-(3,5-difluoro-phenyl)-acetylamino]-pentanoic acid (5-bromo-pyrazin-2-yl)-amide and 1-methoxymethyl-propylamine. The title compound was obtained as a yellow oil. APCI-MS [M+1]=450.3.

### Example 69

### 2-[2-(3,5-Difluoro-phenyl)-acetylamino]-pentanoic Acid (5-isoprorylamino-pyrazin-2-yl)-amide

The title compound was prepared by reacting 2-[2-(3,5-difluoro-phenyl)-acetylamino]-pentanoic acid (5-bromo-pyrazin-2-yl)-amide and isopropylamine. The title compound was obtained as a beige solid. APCI-MS [M+1]=406.2.

### Example 70

### 2-[2-(3,5-Difluoro-phenyl)-acetylamino]-pentanoic Acid [5-(2-hydroxy-ethylamino)-pyrazin-2-yl]amide

The title compound was prepared by reacting 2-[2-(3,5-difluoro-phenyl)-acetylamino]-pentanoic acid (5-bromo-pyrazin-2-yl)-amide and 2-amino-ethanol. The title compound was obtained as a white solid. APCI-MS [M+1]=408.2.

### Example 71

### 2-[2-(3,5-Difluoro-phenyl)-acetylamino]-pentanoic Acid (5-benzylamino-pyrazin-2-yl)-amide

The title compound was prepared by reacting 2-[2-(3,5-difluoro-phenyl)-acetylamino]-pentanoic acid (5-bromo-pyrazin-2-yl)-amide and benzylamine. The title compound was obtained as a white solid. APCI-MS [M+1]=454.3.

### Example 72

### 2-[2-(3,5-Difluoro-phenyl)-acetylamino]-pentanoic Acid [5-(benzyl-ethyl-amino)-pyrazin-2-yl-amide

The title compound was prepared by benzyl-ethyl-amine. The title compound was obtained as a yellow oil. APCI-MS [M+1]=482.3.

### Example 73

### 2-[2-(3,5-Difluoro-phenyl)-acetylamino]-pentanoic Acid (5-pyrrolidin-1-yl-pyrazin-2-yl)-amide

The title compound was prepared by reacting 2-[2-(3,5-difluoro-phenyl)-acetylamino]-pentanoic acid (5-bromo-pyrazin-2-yl)-amide and pyrrolidine. The title compound was obtained as a white solid. APCI-MS [M+1]=418.2.

### Example 74

### 2-[2-(3-Fluoro-5-pyrrolidin-1-yl-phenyl)-acetylamino]-pentanoic Acid (5-pyrrolidin-1-yl-pyrazin-2-yl)-amide

The title compound was prepared by reacting 2-[2-(3,5-difluoro-phenyl)-acetylamino]-pentanoic acid (5-bromo-pyrazin-2-yl)-amide and pyrrolidine. The title compound was obtained as a white solid. APCI-MS (M+1]=469.3.

### Example 75

### 2-[2-(3,5-Difluoro-phenyl)-acetylamino]-pentanoic Acid pyrazin-2-ylamide

The title compound was prepared by reacting 2-[2-(3,5-difluoro-phenyl)-acetylamino]-pentanoic acid (5-bromo-pyrazin-2-yl)-amide and 1-ethyl-propylamine. The title compound was obtained as a yellow oil. APCI-MS [M+1]=349.2.

### Example 76

### 3-(2-(2-[2-(3,5-Difluoro-phenyl)-acetylamino]-pentanoylamino]-pyrimidin-5-yl)-but-2-enoic Acid methyl ester

A mixture of 2-[2-(3,5-difluoro-phenyl)-acetylamino]-pentanoic acid (5-bromo-pyrimidin-2-yl)-amide (110 mg, 0.26 mmol), methyl crotonate (30.9 mg, 0.30 mmol), triethylamine (0.074 ml, 57.3 mg, 0.30 mmol), palladium acetate (3.5 mg, 0.015 mmol), tri-o-tolylphosphine (0.4 mg, 0.031 mmol) in N-methyl-pyrrolodine (1 ml) was heated at reflux overnight. Additional palladium acetate (3.5 mg) and tri-o-tolylphosphine (9.4 mg) was added and the mixture was heated for an additional 20 hr. The mixture cooled to r.t., quenched with water, extracted with ethyl acetate. The organic layer was separated, dried over MgSO4, filtered, concentrated to dryness. The residue wa purified by Shimadzu HPLC to yield the title compound. LC-MS RT=2.2 min, M+1=447.2.

### Example 77

### 3-(6-(2-[2-(3,5-Difluoro-phenyl)-acetylamino]-pentanoylamino]-pyridin-3-yl)-but-2-enoic Acid methyl ester

A mixture of 2-[2-(3,5-difluoro-phenyl)-acetylamino]-pentanoic acid (5-bromo-pyridin-2-yl)-amide (427 mg, 1.0 mmol), methyl crotonate (150 mg, 1.5 mmol), diisopropylethylamine (0.27 ml, 1.54 mmol), palladium acetate (24.7 mg, 0.11 mmol), tri-o-tolylphosphine (54.8 mg, 0.18 mmol) in porpionitrile (15 ml) was heated at reflux overnight. The mixture cooled to r.t., filtered through celite, washed with 10 ml of proprionitrile. The filtrate wwas concentrated to dryness. The residue was quenched with water, extracted with ethyl acetate. The organic layer was separated, dried over MgSO4, filtered, concentrated to dryness to give a brown foam. The residue was purified by Shimadzu HPLC to yield the title compound as a white solid, LC-MS RT=2.6 min, M+1=446.5.

### Example 78

### 2-[2-(3,5-Difluoro-phenyl)-acetylamino]-pentanoic Acid [5-(1-methyl-3-oxo-but-1-enyl)-pyridin-2-yl]-amide

A mixture of 2-[2-(3,5-difluoro-phenyl)-acetylamino]-pentanoic acid (5-bromo-pyridin-2-yl)-amide (426 mg, 1.0 mmol), 3-pentene-2-one (0.21 ml, 1.5 mmol), diisopropylethylamine (0.27 ml, 1.54 mmol), palladium acetate (24.7 mg, 0.11 mmol), tri-o-tolylphosphine (54.8 mg, 0.18 mmol) in porpionitrile (10 ml) was heated at reflux for 5 hr. The mixture cooled to r.t., filtered through celite, washed with 10 ml of proprionitrile. The filtrate wwas concentrated to dryness. The residue was quenched with water, extracted with ethyl acetate. The organic layer was separated, dried over MgSO4, filtered, concentrated to dryness to give 509 mg of the title compound as a brown oil. The residue was purified by silica gel column chromatography using Hexane:EtOAc=1:1 as eluent to give the title compound as a white solid. LC-MS M+1=430.1.

### Example 79

### 2-[2-(3,5-Difluoro-phenyl)-acetylamino]-pentanoic Acid [5-(1-methyl-3-oxo-butyl)-pyridin-2-yl]-amide

A mixture of 2-[2-(3,5-difluoro-phenyl)-acetylamino]-pentanoic acid [5-(1-methyl-3-oxo-but-1-enyl)-pyridin-2-yl]-amide (1.55 g) and PtO2 (300 mg) in EtOAc (20 ml) was hydrogenated at 45 psi at r.t. for 4 hr. the mixture was filtered through celite. The filtrate was concentrated to dryness, purified by silica gel column chromatography using hexane:EtOAc=1:1 as eluent to give a mixture of two isomers of the title compound as an oil. LC-MS RT=2.5 min, M+1=431.9. The two diastereoisomers were then separated well by chiral HPLC.

### Example 80

### 2-[2-(3,5-Difluoro-phenyl)-acetylamino]-pentanoic Acid [5-(pyrazin-2-ylamino)-pyrazin-2-yl]-amide

2-[2-(3,5-Difluoro-phenyl)-acetylamino]-pentanoic acid (5-bromo-pyrazin-2-yl)-amide (427 mg, 1.0 mmol), pent-3-en-2-one (126 mg, 1.5 mmol), palladium (II) acetate (25 mg, 0.11 mmol), tri-o-tolyl-phosphine (54 mg, 0.18 mmol), and ethyl-diisopropyl-amine (198 mg, 1.5 mmol) were stirred in propionitrile heated to reflux for 4 hours, then stirred at room temperature overnight. The mixture was filtered through Celite, washed with propionitrile, and concentrated. The resulting oil was quenched with water and extracted with ethyl acetate. The organic layer was concentrated and purified by preparative HPLC to give title compound as a yellow solid. APCI-MS [M+1]=442.0.

### Example 81

### 2-[2-(3,5-Difluoro-phenyl)-acetylamino]-pentanoic Acid [5-(3-oxo-butyl)-pyrazin-2-yl]-amide

The title compound was prepared by the method analogous to that described in Example 81 by reacting 2-[2-(3,5-difluoro-phenyl)-acetylamino]-pentanoic acid (5-bromo-pyrazin-2-yl)-amide, but-3-en-2-one, palladium (II) acetate, tri-o-tolyl-phosphine, and ethyl-diisopropyl-amine. The title compound was obtained as a yellow solid. APCI-MS [M+l]=419.1.

### Example 82

### 2-[2-(3,5-Difluoro-phenyl)-acetylamino]-pentanoic Acid [5-(1-hydroxy-1-methyl-ethyl)-pyrazin-2-yl]-amide

2-[2-(3,5-Difluoro-phenyl)-acetylamino]-pentanoic acid (5-bromo-pyrazin-2-yl)-amide (200 mg, 0.47 mmol) was dissolved in tetrahydrofuran and cooled with a dry acetone bath. N-butyllithium (0.75 mL of a 2.5 M solution in hexane, 1.9 mmol) was quickly added to the mixture, followed immediately by acetone (1 mL, 13.6 mmol) and left to stir for 20 minutes. The mixture was then quenched with water and extracted with ethyl acetate. The organic layer was concentrated and purified by preparative HPLC to give the title compound as a yellow oil. APCI-MS [M+1]=407.2.

### Example 83

### 2-[2-(3,5-Difluoro-phenyl)-acetylamino]-pentanoic Acid [5-(3-amino-1-methyl-butyl)-pyridin-2-yl]-amide

A mixture of 2-[2-(3,5-difluoro-phenyl)-acetylamino]-pentanoic acid [5-(1-methyl-3-oxo-butyl)-pyridin-2-yl]-amide (125 mg, 0.29 mmol), 2 M amonium in methanol (1.0 ml, 2.0 mmol), AcOH (0.125 ml), sodium acetate (125 mg), in dichloroethane was stirred at r.t. for 1 hr. NaBH3CN (125 mg, 2.01 mmol) was added and the resulting mixture was heated at 45° C. for 4 hr and LC-MS indicated all starting material consumed. The mixture was cooled to r.t. and quenched with water and extracted with methylene chloride. The organic layer was basified with dilute NaOH, washed with brine, separated, dried over MgSO4, filtered. The filtrate was concentrated and purified by Shimadzu HPLC to give the desired product as a white solid, LC-MS M+1=433.0.0.

### Example 84

### 2-[2-(3,5-Difluoro-phenyl)-acetylamino]-pentanoic Acid [5-(1-methyl-3-methylamino-butyl)-pyridin-2-yl]-amide

A mixture of 2-[2-(3,5-difluoro-phenyl)-acetylamino]-pentanoic acid [5-(1-methyl-3-oxo-butyl)-pyridin-2-yl]-amide (125 mg, 0.29 mmol), 2 M monomethylamine in THF (1.0 ml, 2.0 mmol), AcOH (0.125 ml), sodium acetate (125 mg), in dichloroethane was stirred at r.t. for 1 hr. NaBH3CN (125 mg, 2.01 mmol) was added and the resulting mixture was heated at 45° C. for 4 hr and LC-MS indicated all starting material consumed. The mixture was cooled to r.t. and quenched with water and extracted with methylene chloride. The organic layer was basified with dilute NaOH, washed with brine, separated, dried over MgSO4, filtered. The filtrate was concentrated and purified by Shimadzu HPLC to give the desired product as a white solid, LC-MS M+1=447.0.

### Example 85

### 2-[2-(3,5-Difluoro-phenyl)-acetylamino]-pentanoic Acid {5-[3-(2-hydroxy-ethylamino)-1-methyl-butyl]-pyridin-2-yl}-amide

A mixture of 2-[2-(3,5-difluoro-phenyl)-acetylamino]-pentanoic acid [5-(1-methyl-3-oxo-butyl)-pyridin-2-yl]-amide (125 mg, 0.29 mmol), ethanol amine (0.12 ml, 2.0 mmol), AcOH (0.125 ml), sodium acetate (125 mg), in dichloroethane was stirred at r.t. for 1 hr. NaBH3CN (125 mg, 2.01 mmol) was added and the resulting mixture was heated at 45° C. for 4 hr and LC-MS indicated all starting material consumed. The mixture was cooled to r.t. and quenched with water and extracted with methylene chloride. The organic layer was basified with dilute NaOH, washed with brine, separated, dried over MgSO4, filtered. The filtrate was concentrated and purified by Shimadzu HPLC to give the desired product as a white solid, LC-MS M+1=477.3.

### Example 86

### 2-[2-(3,5-Difluoro-phenyl)-acetylamino]-pentanoic Acid {5-[1-methyl-3-(2,2,2-trifluoro-ethylamino)-butyl]-pyridin-2-yl}-amide

A mixture of 2-[2-(3,5-difluoro-phenyl)-acetylamino]-pentanoic acid [5-(1-methyl-3-oxo-butyl)-pyridin-2-yl]-amide (125 mg, 0.29 mmol), trifluoroethylamine (0.16 ml, 2.0 mmol)), AcOH (0.125 ml), sodium acetate (125 mg), in dichloroethane-was stirred at r.t. for 1 hr. NaBH3CN (125 mg, 2.01 mmol) was added and the resulting mixture was heated at 45° C. for 4 hr and LC-MS indicated all starting material consumed. The mixture was cooled to r.t. and quenched with water and extracted with methylene chloride. The organic layer was basified with dilute NaOH, washed with brine, separated, dried over MgSO4, filtered. The filtrate was concentrated and purified by Shimadzu HPLC to give a mixture of four diasteroeoisomers of the title compound as a white solid, LC-MS M+1=515.2. The four diastereoisomers were separated by chiral HPLC to provide pure of each following four isomers:
2-[2-(3,5-Difluoro-phenyl)-acetylamino]-(S)-pentanoic acid {5-[1-(S)-methyl-3-(S)-(2,2,2-trifluoro-ethylamino)-butyl]-pyridin-2-yl}-amide:
2-[2-(3,5-Difluoro-phenyl)-acetylamino]-(S)-pentanoic acid {s5-[—(S)-methyl-3-(R)-(2,2,2-trifluoro-ethylamino)-butyl]-pyridin-2-yl}-amide;
2-[2-(3,5-Difluoro-phenyl)-acetylamino]-(S)-pentanoic acid {5-[1-(R)-methyl-3-(S)-(2,2,2-trifluoro-ethylamino)-butyl]-pyridin-2-yl}-amide:
2-[2-(3,5-Difluoro-phenyl)-acetylamino]-(S)-pentanoic acid {5-[1-(R)-methyl-3-(R)-(2,2,2-trifluoro-ethylamino)-butyl]-pyridin-2-yl}-amide.

### Example 87

### 2-[2-(3,5-Difluoro-phenyl)-acetylamino]-pentanoic Acid [5-(3-hydroxy-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (500 mg) and NaBH4 (500 mg) in methanol (10 ml) was stirred at r.t. for 30 min. The mixture was quenched with water, dilute HCl, concentrated to remove methanol and extracted with ethyl acetate. The organic layer was separated, dried over MgSO4, filtered. The filtrate was concentrated to dryness. The residue was purified by HPLC to give 407 mg of the title compound as a colorless glass foam, LC-MS RT=2.1 min, M+1=420.0.

### Example 88

### 2-[2-(3,5-Difluoro-phenyl)-acetylamino]-pentanoic Acid [5-(3-hydroxy-1-methyl-butyl)-pyridin-2-yl]-amide

The title compound was prepared by the method analogous to that described in Example 87 as a colorless glass foam, LC-MS RT=2.4 min, M+1=434.0.

### Example 89

### 2-[2-(3,5-Difluoro-phenyl)-acetylamino]-pentanoic Acid {5-[3-(2,2,2-trifluoro-ethylamino)-butyl]-pyridin-2-yl}-amide

A mixture of 2-[2-(3,5-difluoro-phenyl)-acetylamino]-pentanoic acid [5-(3-oxo-butyl)-pyridin-2-yl]-amide (76 mg, 0.18 mmol), trifluoroethylamine (36 mg, 0.36 mmol), acetic acid (0.01 ml, 0.18 mmol), sodium triacetoxyborohydride (76 mg, 0.36 mmol) in dichloroethane was stirred at r.t. overnight. The mixture was quenched with water, basified to pH 9-10, extracted with methylene chloride. The organic layer was separated, dried over MgSO4, and filtered. The filtrate was concentrated to dryness to give a gummy solid. The solid was converted to the corresponding HCl salt by adding 4N HCl in dioxane, then concentrated to dryness to give a white solid after trituration with hexane. LC-MS RT=2.1 min, M+1=500.9.

### Example 90

### 3-(5-[2-[2-(3,5-Difluoro-phenyl)-acetylamino]-pentanoylamino]-pyrazin-2-methylamino)-acetic acid methyl ester

2-[2-(3,5-Difluoro-phenyl)-acetylamino]-pentanoic acid (5-bromo-pyrazin-2-yl)-amide (100 mg, 0.23 mmol), methylamino-acetic acid methyl ester (94 mg, 0.47 mmol), and ethyl-diisopropyl-amine (0.5 mL, 2.9 mmol) were stirred in dimethyl sulfoxide heated to 100° C. for 3 hours. The mixture was quenched with water and extracted with ethyl acetate. The organic layer was concentrated and purified by preparative HPLC to give the title compound as a yellow oil. APCI-MS [M+1]=450.1.

### Example 91

### 2-[2-(3,5-Difluoro-phenyl)-acetylamino]-pentanoic Acid [5-(3-oxo-but-1-enyl)-pyridin-2-yl]-amide

A mixture of 2-[2-(3,5-difluoro-phenyl)-acetylamino]-pentanoic acid (5-bromo-pyridin-2-yl)-amide (426 mg, 1.0 mmol), methylvinyl ketone (0.13 ml, 1.5 mmol), diisopropylethylamine (0.27 ml, 1.54 mmol), palladium acetate (24.7 mg, 0.11 mmol), tri-o-tolylphosphine (54.8 mg, 0.18 mmol) in porpionitrile (10 ml) was heated at reflux for 5 hr. The mixture cooled to r.t., filtered through celite, washed with 10 ml of proprionitrile. The filtrate wwas concentrated to dryness. The residue was quenched with water, extracted with ethyl acetate. The organic layer was separated, dried over MgSO4, filtered, concentrated to dryness to give 509 mg of the title compound as a brown oil. The residue was purified by silica gel column chromatography using hexane:EtOAc=4:1 to hexane:EtOac=1:1 as eluent to give the title compound as a white solid. LC-MS RT=2.4 min, M+1=415.9.

### Example 92

### 3-(6-(2-[2-(3,5-Difluoro-phenyl)-acetylamino]-pentanoylamino]-pyridin-3-yl)-propionic Acid methyl ester

A mixture of 3-(6-{2-[2-(3,5-difluoro-phenyl)-acetylamino]-pentanoylamino}-pyridin-3-yl)-acrylic acid methyl ester (140 mg, 0.325 mmol) and PtO2 (20 mg) in EtOAc (120 ml) was hydrogenated at 50 psi at r.t. for 4 hr. The mixture was filtered through celite. The filtrate was concentrated to dryness, purified by silica gel column chromatography using hexane:EtOAc=1:1 as eluent to gve the title compound as a white solid. LC-MS RT=2.5 min, M+1=433.9.

### Example 93

### 2-[2-(3,5-Difluoro-phenyl)-acetylamino]-pentanoic Acid [5-(3-oxo-butyl)-pyridin-2-yl]-amide

A mixture of 2-[2-(3,5-difluoro-phenyl)-acetylamino]-pentanoic acid [5-(3-oxo-but-1-enyl)-pyridin-2-yl]-amide (175 mg, 0.42 mmol) and PtO2 (35 mg) in EtOAc (10 ml) was hydrogenated at 50 psi at r.t. for 2 hr. the mixture was filtered through celite. The filtrate was concentrated to dryness, purified by silica gel column chromatography using hexane:EtOAc=1:1 as eluent to gve the title compound as a white solid. LC-MS RT=2.4 min, M+1=417.9.

### Example 94

### 2-[2-(3,5-Difluoro-phenyl)-acetylamino]-pentanoic Acid [5-(1-hydroxy-ethyl)-pyridin-2-yl]-amide

A mixture of 2-[2-(3,5-difluoro-phenyl)-acetylamino]-pentanoic acid (5-acetyl-pyridin-2-yl)-amide (22 mg, 0.057 mmol) and sodium borohydride (10 mg, 0.26 mmol) in methanol was stirred at room temperature for 10 minutes. The mixture was quenched with water and extracted with ethyl acetate. The organic layer was concentrated to give the title compound as a white solid. APCI-MS [M+1]=392.1.

### Example 95

### 2-[2-(3,5-Difluoro-phenyl)-acetylamino]-pentanoic Acid (5-hydroxymethyl-pyridin-2-yl)-amide

A mixture of 6-{2-[2-(3,5-difluoro-phenyl)-acetylamino]-pentanoylamino}-nicotinic acid methyl ester and 5 eq of sodium borohydride in methanol was stirred at r.t. for 10 min. The mixture was quenched with water and extracted with ethyl acetate. The organic layer was concentrated to give the title compound as a white solid. LC-MS [M+1]=377.9 RT=1.9 min.

### Example 96

### 2-[2-(3,5-Difluoro-phenyl)-acetylamino]-pentanoic Acid [5-(3-methylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (83 mg, 0.2 mmol), 2.0M methylamine in THF (0.3 ml, 0.6 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give an oil, LC-MS R.T. 1.8 min, M+1=433.0 The corresponding HCl salt was prepared by adding 4N HCl in dioxane, then concentrated to dryness to give a white solid after trituration with hexane.

### Example 97

### 2-[2-(3,5-Difluoro-phenyl)-acetylamino]-pentanoic Acid [5-(3-ethylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (83 mg, 0.2 mmol), 2.0M ethylamine in THF (0.3 ml, 0.6 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give an oil, LC-MS R.T. 1.8 min, M+1=447.0 The corresponding HCl salt was prepared by adding 4N HCl in dioxane, then concentrated to dryness to give a white solid after trituration with hexane.

### Example 98

### 2-[2-(3,5-Difluoro-phenyl)-acetylamino]-pentanoic Acid [5-(3-propylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (83 mg, 0.2 mmol), propylamine (23.6 mg, 0.4 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give 79.4 mg of the title compound as an oil, LC-MS R.T. 2.0 min, M+1=461.0. The corresponding HCl salt was prepared by adding 4N HCl in dioxane, then concentrated to dryness to give 88 mg of a white solid after trituration with hexane.

### Example 99

### 2-[2-(3,5-Difluoro-phenyl)-acetylamino]-pentanoic Acid [5-(3-isopropylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (83 mg, 0.2 mmol), isopropylamine (23.6 mg, 0.4 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give 81 mg of the title compound as an oil, LC-MS R.T. 1.9 min, M+1=461.0. The corresponding HCl salt was prepared as a white solid (85 mg) by adding 4N HCl in dioxane, then concentrated to dryness and triturated with hexane.

### Example 100

### 2-[2-(3,5-Difluoro-phenyl)-acetylamino]-pentanoic Acid [5-(3-butylamino-butyl)-pyridin-2-yl-amide

A mixture of 2-[2-(3,5-Difluoro-phenyl)-acetylamino]-pentanoic acid [5-(3-oxo-butyl)-pyridin-2-yl]-amide (83 mg, 0.2 mmol), n-butylamine (29.3 mg, 0.4 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give 110 mg of the title compound as an oil. The mixture was purified by HPLC to give 50 mg of the title compound as a white solid of the formic acid salt. LC-MS RT=2.0 min, M+1=475.0.

### Example 101

### 2-[2-(3,5-Difluoro-phenyl)-acetylamino]-pentanoic Acid [5-(3-isobutylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (83 mg, 0.2 mmol), n-isobutylamine (29.3 mg, 0.4 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give 111 mg of the title compound as an oil. The mixture was purified by HPLC to give 54 mg of the formic acid salt of the title compound as a white solid. LC-MS RT=2.0 min, M+1=475.0.

### Example 102

### 2-[2-(3,5-Difluoro-phenyl)-acetylamino]-pentanoic Acid {5-[3-(3-methyl-butylamino)-butyl]-pyridin-2-yl}-amide

A mixture of 2-[2-(3,5-difluoro-phenyl)-acetylamino]-pentanoic acid [5-(3-oxo-butyl)-pyridin-2-yl]-amide (83 mg, 0.2 mmol), n-isoamylamine (34.9 mg, 0.4 mmol), acetic acid (0.014 ml, 0.2 mmol), sodium triacetoxyborohydride (83.5 mg, 0.395 mmol) in dichloroethane (1.5 ml) was stirred at r.t. overnight. The mixture was quenched with water, basified to pH 9-10, extracted with methylene chloride. The organic layer was separated, dried over MgSO4, and filtered. The filtrate was concentrated to dryness to give 90 mg of the title compound as an oil. The mixture was purified by HPLC to give 52 mg of the formic acid salt of the title compound as a white solid. LC-MS RT=2.1 min, M+1=489.0.

### Example 103

### 2-[2-(3,5-Difluoro-phenyl)-acetylamino]-pentanoic Acid {5-[1-(3-methyl-butylamino)-ethyl]-pyridin-2-yl}-amide

2-[2-(3,5-Difluoro-phenyl)-acetylamino]-pentanoic acid (5-acetyl-pyridin-2-yl)-amide (47 mg, 0.12 mmol), 3-methyl-butylamine (21 mg, 0.24 mmol), sodium triacetoxyborohydride (47 mg, 0.22 mmol), and acetic acid (7.3 mg, 0.12 mmol) were stirred in 1,2-dichloroethane at room temperature overnight. An additional 21 mg of 3-methyl-butylamine, 47 mg of sodium triacetoxyborohydride, and 10 μL of acetic acid were added to the mixture and stirred overnight at room temperature. The mixture was quenched with dilute sodium hydroxide and extracted with dichloromethane. The organic layer was concentrated and purified by preparative HPLC to give the title compound as a white solid. LC-MS [M+1]=461.0 RT=2.0 min.

### Example 104

### 6-[2-[2-(3,5-Difluoro-phenyl)-acetylamino]-pentanoylamino]-nicotinic Acid

6-{2-[2-(3,5-Difluoro-phenyl)-acetylamino]-pentanoylamino}-nicotinic acid methyl ester (100 mg, 0.24 mmol) and DIBAL-H (0.42 mL of a 1.5 M solution in toluene, 0.64 mmol) were stirred in tetrahydrofuran cooled in a dry acetone bath for 90 minutes. The mixture was quenched with water and extracted with ethyl acetate. The organic layer was filtered through Celite and washed with methanol. The organic mixture was then concentrated and purified by preparative HPLC to give the title compound as a clear oil. LC-MS [M+1]=392.0, RT=2.2 min.

### Example 105

### 2-[2-(3,5-Difluoro-phenyl)-acetylamino]-pentanoic Acid [5-(1-isobutylamino-ethyl)-pyridin-2-yl]-amide

2-[2-(3,5-Difluoro-phenyl)-acetylamino]-pentanoic acid (5-acetyl-pyridin-2-yl)-amide (70 mg, 0.18 mmol), isobutylamine (26 mg, 0.36 mmol), sodium triacetoxyborohydride (70 mg, 0.33 mmol), and acetic acid (11 mg, 0.18 mmol) were stirred in 1,2-dichloroethane at room temperature overnight. An additional 60 μL of isobutylamine, 70 mg of sodium triacetoxyborohydride, and 10 μL of acetic acid were added to the mixture with 0.2 mL of dimethylformamide and left to stir for 3 days at room temperature. The mixture was quenched with dilute sodium hydroxide and extracted with ethyl acetate. The organic layer was concentrated and purified by preparative HPLC to give the title compound as a white glass-form. APCI-MS [M+1]=447.2.

### Example 106

### 2-[2-(3,5-Difluoro-phenyl)-acetylamino]-pentanoic Acid {5-[1-(3,3-dimethyl-butylamino)-ethyl]-pyridin-2-yl}-amide

2-[2-(3,5-Difluoro-phenyl)-acetylamino]-pentanoic acid (5-acetyl-pyridin-2-yl)-amide (70 mg, 0.18 mmol), 3,3-dimethyl-butylamine (0.36 mmol), sodium triacetoxyborohydride (70 mg, 0.33 mmol), and acetic acid (11 mg, 0.18 mmol) were stirred in 1,2-dichloroethane at room temperature overnight. An additional 60 jiL of 3,3-dimethyl-butylamine, 70 mg of sodium triacetoxyborohydride, and 10 μL of acetic acid were added to the mixture with 0.2 mL of dimethylformamide and left to stir for 3 days at room temperature. The mixture was quenched with dilute sodium hydroxide and extracted with ethyl acetate. The organic layer was concentrated and purified by preparative HPLC to give the title compound as a white solid. APCI-MS [M+1]=475.2.

### Example 107

### 2-[2-(3,5-Difluoro-phenyl)-acetylamino]-pentanoic Acid {5-[1-(1-ethyl-propylamino)-ethyl]-pyridin-2-yl}-amide

2-[2-(3,5-Difluoro-phenyl)-acetylamino]-pentanoic acid (5-acetyl-pyridin-2-yl)-amide (48 mg, 0.12 mmol), 1-ethyl-propylamine (21 mg, 0.25 mmol), sodium triacetoxyborohydride (48 mg, 0.23 mmol), and acetic acid (7.5 mg, 0.12 mmol) were stirred in dichloromethane at room temperature for 3 days. An additional 21 mg of 1-ethyl-propylamine, 48 mg of sodium triacetoxyborohydride, and 7.5 mg of acetic acid were added to the mixture and stirred at room temperature for 2 days. Sodium cyanoborohydride (15.5 mg, 0.25 mmol) was added to the mixture and stirred at room temperature overnight. The mixture was then quenched with dilute sodium hydroxide and extracted with dichloromethane. The organic layer was concentrated and purified by preparative HPLC to give the title compound as a clear oil. APCI-MS [M+1]=461.2.

### Example 108

### 2-[2-(3,5-Difluoro-phenyl)-acetylamino]-pentanoic Acid {5-[1-(2,2,2-trifluoro-ethylamino)-ethyl-pyridin-2-yl}-amide

2-[2-(3,5-Difluoro-phenyl)-acetylamino]-pentanoic acid (5-acetyl-pyridin-2-yl)-amide (100 mg, 0.26 mmol), 2,2,2-trifluoro-ethylamine (153 mg, 1.5 mmol), sodium triacetoxyborohydride (100 mg, 0.47 mmol), and acetic acid (53 mg, 0.88 mmol) were stirred in dichloromethane/dimethylformamide at room temperature overnight. Sodium cyanoborohydride (100 mg, 1.6 mmol) was added to the mixture and stirred at room temperature overnight, then at 40° C. for 1 hour, then room temperature overnight. The mixture was quenched with dilute sodium hydroxide and extracted with dichloromethane. The organic layer was concentrated and purified by preparative HPLC to give the title compound as a white glass form. APCI-MS [M+1]=473.1.

The following examples 109-120 were prepared by the method analogous to that described in Example 108 starting from 2-[2-(3,5-difluoro-phenyl)-acetylamino]-pentanoic acid (5-acetyl-pyridin-2-yl)-amide and an appropriate amine in the presence of a reducing agent.

### Example 109

### 2-[2-(3,5-Difluoro-phenyl)-acetylamino]-pentanoic Acid [5-(1-butylamino-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, butylamine, sodium triacetoxyborohydride, sodium cyanoborohydride, and acetic acid. The title compound was obtained as a white glass form. APCI-MS [M+1]=447.1.

### Example 110

### 2-[2-(3,5-Difluoro-phenyl)-acetylamino]-pentanoic Acid [5-(1-morpholin-4-yl-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, morpholine, sodium triacetoxyborohydride, sodium cyanoborohydride, and acetic acid. The title compound was obtained as a white glass form. APCI-MS [M+1]=461.1.

### Example 111

### 2-[2-(3,5-Difluoro-phenyl)-acetylamino]-pentanoic Acid [5-(1-benzylamino-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, benzylamine, sodium triacetoxyborohydride, sodium cyanoborohydride, and acetic acid. The title compound was obtained as a white glass form. APCI-MS [M+1]=481.1.

### Example 112

### 2-[2-(3,5-Difluoro-phenyl)-acetylamino]-pentanoic Acid [5-(1-cyclopropylamino-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, cyclopropylamine, sodium triacetoxyborohydride, sodium cyanoborohydride, and acetic acid. The title compound was obtained as a white glass form. APCI-MS [M+1]=431.1.

### Example 113

### 2-[2-(3,5-Difluoro-phenyl)-acetylamino]-pentanoic Acid [5-(1-cyclopropylamino-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, cyclopropylamine, sodium triacetoxyborohydride, sodium cyanoborohydride, and acetic acid. The title compound was obtained as a white glass form. APCI-MS [M+1]=431.1.

### Example 114

### 2-[2-(3,5-Difluoro-phenyl)-acetylamino]-pentanoic Acid {5-[1-(1-benzyl-pyrrolidin-3-ylamino)-ethyl]-pyridin-2-yl}-amide

The title compound was prepared by reacting 2-[2-(3,5-difluoro-phenyl)-acetylamino]-pentanoic acid (5-acetyl-pyridin-2-yl)-amide, 1-benzyl-pyrrolidin-3-ylamine, sodium triacetoxyborohydride, sodium cyanoborohydride, and acetic acid. The title compound was obtained as a white glass form. APCI-MS [M+1]=550.2.

### Example 115

### 2-[2-(3,5-Difluoro-phenyl)-acetylamino]-pentanoic Acid {5-[1-(1-benzyl-pyrrolidin-3-ylamino)-ethyl]-pyridin-2-yl}-amide

The title compound was prepared by reacting 2-[2-(3,5-difluoro-phenyl)-acetylamino]-pentanoic acid (5-acetyl-pyridin-2-yl)-amide, 1-benzyl-pyrrolidin-3-ylamine, sodium triacetoxyborohydride, sodium cyanoborohydride, and acetic acid. The title compound was obtained as a clear oil. APCI-MS [M+1]=550.2.

### Example 116

### 2-[2-(3,5-Difluoro-phenyl)-acetylamino]-pentanoic Acid [5-(1-cyclobutylamino-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, cyclobutylamine, sodium triacetoxyborohydride, sodium cyanoborohydride, and acetic acid. The title compound was obtained as a white glass form. APCI-MS [M+1l=445.1.

### Example 117

### 2-[2-(3,5-Difluoro-phenyl)-acetylamino]-pentanoic Acid [5-(1-pyrrolidin-1-yl-ethyl)-pyridin-2-yl]-amide

The title compound was prepared by reacting 2-[2-(3,5-difluoro-phenyl)-acetylamino]-pentanoic acid (5-acetyl-pyridin-2-yl)-amide, pyrrolidine, sodium triacetoxyborohydride, sodium cyanoborohydride, and acetic acid. The title compound was obtained as a white glass form. APCI-MS [M+1]=445.3.

### Example 118

### 2-[2-(3,5-Difluoro-phenyl)-acetylamino]-pentanoic Acid {5-[1-(4-methyl-piperazin-1-yl)-ethyl]-pyridin-2-yl}-amide

The title compound was prepared by reacting 2-[2-(3,5-difluoro-phenyl)-acetylamino]-pentanoic acid (5-acetyl-pyridin-2-yl)-amide, 1-methyl-piperazine, sodium triacetoxyborohydride, sodium cyanoborohydride, and acetic acid. The title compound was obtained as a white glass form. LC-MS [M+1]=474.0, RT=1.8 min.

### Example 119

### 2-[2-(3,5-Difluoro-phenyl)-acetylamino]-pentanoic Acid {5-[1-(4-methyl-piperazin-1-yl)-ethyl]-pyridin-2-yl}-amide

The title compound was prepared by reacting 2-[2-(3,5-difluoro-phenyl)-acetylamino]-pentanoic acid (5-acetyl-pyridin-2-yl)-amide, 1-methyl-piperazine, sodium triacetoxyborohydride, sodium cyanoborohydride, and acetic acid. The title compound was obtained as a white glass form. LC-MS [M+1]=474.0, RT=1.9 min.

### Example 120

### 2-[2-(3,5-Difluoro-phenyl)-acetylamino]-pentanoic Acid {5-[1-(2-hydroxy-ethylamino)-ethyl]-pyridin-2-yl}-amide

The title compound was prepared by reacting 2-[2-(3,5-difluoro-phenyl)-acetylamino]-pentanoic acid (5-acetyl-pyridin-2-yl)-amide, 2-amino-ethanol, sodium triacetoxyborohydride, sodium cyanoborohydride, and acetic acid. The title compound was obtained as a white glass form. APCI-MS [M+1]=435.2.

### Example 121

### 2-[2-(3,5-Difluoro-phenyl)-acetylamino]-pentanoic Acid [5-(3-morpholin-4-yl-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (100 mg, 0.24 mmol), n-morpholine (41.7 mg, 0.48 mmol), acetic acid (0.014 ml, 0.24 mmol), sodium triacetoxyborohydride (100 mg, 0.47 mmol) in dichloroethane (2 ml) was stirred at r.t. overnight. The mixture was quenched with water, basified to pH 9-10, extracted with methylene chloride. The organic layer was separated, dried over MgSO4, and filtered. The filtrate was concentrated to dryness to give 114 mg of the title compound as an oil. The mixture was purified by HPLC to give the formic acid salt of the title compound as a white solid. LC-MS RT=1.6 min, M+1=489.2.

### Example 122

### 2-[2-(3,5-Difluoro-phenyl)-acetylamino]-pentanoic Acid [5-(3-pyrrolidin-1-yl-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (100 mg, 0.24 mmol), n-pyrrolidine (35.0 mg, 0.48 mmol), acetic acid (0.014 ml, 0.24 mmol), sodium triacetoxyborohydride (100 mg, 0.47 mmol) in dichloroethane (2 ml) was stirred at r.t. overnight. The mixture was quenched with water, basified to pH 9-10, extracted with methylene chloride. The organic layer was separated, dried over MgSO4, and filtered. The filtrate was concentrated to dryness to give 92 mg of the title compound as an oil. The mixture was purified by HPLC to give 44 mg of the formic acid salt of the title compound as a white solid. LC-MS RT=1.7 min, M+1=473.2.

### Example 123

### 2-[2-(3,5-Difluoro-phenyl)-acetylamino]-pentanoic Acid [5-(3-benzylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (100 mg, 0.24 mmol), n-benzylamine (51.3 mg, 0.48 mmol), acetic acid (0.014 ml, 0.24 mmol), sodium triacetoxyborohydride (100 mg, 0.47 mmol) in dichloroethane (2 ml) was stirred at r.t. overnight. The mixture was quenched with water, basified to pH 9-10, extracted with methylene chloride. The organic layer was separated, dried over MgSO4, and filtered. The filtrate was concentrated to dryness to give 120 mg of the title compound as an oil. The mixture was purified by HPLC to give 71 mg of the formic acid salt of the title compound as a white solid. LC-MS RT=1.86 min, M+1=509.2.

### Example 124

### 2-[2-(3,5-Difluoro-phenyl)-acetylamino]-pentanoic Acid [5-(3-cyclopropylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (100 mg, 0.24 mmol), n-cyclopropylamine (0.033 ml, 0.48 mmol), acetic acid (0.014 ml, 0.24 mmol), sodium triacetoxyborohydride (100 mg, 0.47 mmol) in dichloroethane (2 ml) was stirred at r.t. overnight. The mixture was quenched with water, basified to pH 9-10, extracted with methylene chloride. The organic layer was separated, dried over MgSO4, and filtered. The filtrate was concentrated to dryness to give 113 mg of the title compound as an oil. The mixture was purified by HPLC to give 60 mg of the formic acid salt of the title compound as a white solid. LC-MS RT=1.67 min, M+1=459.2.

### Example 125

### 2-[2-(3,5-Difluoro-phenyl)-acetylamino]-pentanoic Acid [5-(3-cyclobutylamino-butyl)-pyridin-2-yl]-amide

A mixture of 2-[2-(3,5-difluoro-phenyl)-acetylamino]-pentanoic acid [5-(3-oxo-butyl)-pyridin-2-yl]-amide (100 mg, 0.24 mmol), n-cyclobutylamine (0.041 ml, 0.48 mmol), acetic acid (0.014 ml, 0.24 mmol), sodium triacetoxyborohydride (100 mg, 0.47 mmol) in dichloroethane (2 ml) was stirred at r.t. overnight. The mixture was quenched with water, basified to pH 9-10, extracted with methylene chloride. The organic layer was separated, dried over MgSO4, and filtered. The filtrate was concentrated to dryness to give 109 mg of the title compound as an oil. The mixture was purified by HPLC to give 45 mg of the formic acid salt of the title compound as a white solid. LC-MS RT=1.9 min, M+1=473.0.

The following examples 126-148 were prepared by the method analogous to that described in Example 125 using reductive amination method.

### Example 126

### 2-[2-(3,5-Difluoro-phenyl)-acetylamino]-pentanoic Acid [5-(3-(S)-cyclopropylamino-butyl)-pyridin-2-yl]-amide

The title compound was prepared as a white solid, LC-MS M+1=459.0, RT=1.9 min.

### Example 127

### 2-[2-(3,5-Difluoro-phenyl)-acetylamino]-pentanoic Acid [5-(3-(R)-cyclopropylamino-butyl)-pyridin-2-yl]-amide

The title compound was prepared as a white solid, LC-MS M+1=459.0, RT=1.8 min.

### Example 128

### 2-[2-(3,5-Difluoro-phenyl)-acetylamino]-pentanoic Acid [5-(3-oxo-propenyl)-pyridin-2-yl]-amide

The title compound was prepared as a white solid, LC-MS M+1=401.9, RT=2.3 min.

### Example 129

### 2-[2-(3,5-Difluoro-phenyl)-acetylamino]-pentanoic Acid [5-(3-cyclobutylamino-butyl)-pyridin-2-yl]-amide

The title compound was prepared as a white solid, LC-MS M+1=473.0, RT=1.9 min.

### Example 130

### 2-[2-(3,5-Difluoro-phenyl)-acetylamino]-pentanoic Acid [5-(3-cyclobutylamino-butyl)-pyridin-2-yl]-amide

The title compound was prepared as a white solid, LC-MS M+1=4739.0, RT=2.0 min.

### Example 131

### 2-[2-(3,5-Difluoro-phenyl)-acetylamino]-pentanoic Acid [5-(S)-(3-benzylamino-1-methyl-butyl)-pyridin-2-yl]-amide

The title compound was prepared as a white solid, LC-MS M+1=523.0, RT=2.2 min.

### Example 132

### 2-[2-(3,5-Difluoro-phenyl)-acetylamino]-pentanoic Acid [5-(R)-(3-benzylamino-1-methyl-butyl)-pyridin-2-yl]-amide

The title compound was prepared as a white solid, LC-MS M+1=523.0, RT=2.2 min.

### Example 133

### 2-[2-(3,5-Difluoro-phenyl)-acetylamino]-pentanoic Acid {5-[3-(1-(S)-phenyl-ethylamino)-butyl]-pyridin-2-yl}-amide

The title compound was prepared as a white solid, LC-MS M+1=523.0, RT=2.1 min.

### Example 134

### 2-[2-(3,5-Difluoro-phenyl)-acetylamino]-pentanoic Acid {5-[3-(1-(R)-phenyl-ethylamino)-butyl]-pyridin-2-yl}-amide

The title compound was prepared as a white solid, LC-MS M+1=523.0, RT=2.1 min.

### Example 135

### 2-[2-(3,5-Difluoro-phenyl)-acetylamino]-pentanoic Acid {5-[3-(2-hydroxy-1-phenyl-ethylamino)-butyl]-pyridin-2-yl}-amide

The title compound was prepared as a white solid, LC-MS M+1=539.0.

### Example 136

### 2-[2-(3,5-Difluoro-phenyl)-acetylamino]-pentanoic Acid {5-[3-(2-hydroxy-1-phenyl-ethylamino)-butyl]-pyridin-2-yl]-amide

The title compound was prepared as a white solid, LC-MS M+1=538.9.

### Example 137

### 2-[2-(3,5-Difluoro-phenyl)-acetylamino]-pentanoic Acid {5-[3-(2-trifluoromethyl-benzylamino)-butyl]-pyridin-2-yl}-amide

The title compound was prepared as a white solid, LC-MS M+1=577.0.

### Example 138

### 2-[2-(3,5-Difluoro-phenyl)-acetylamino]-pentanoic Acid (5-[3-(3-trifluoromethyl-benzylamino)-butyl]-pyridin-2-yl}-amide

The title compound was prepared as a white solid, LC-MS M+1=577.1.

### Example 139

### 2-[2-(3,5-Difluoro-phenyl)-acetylamino]-pentanoic Acid (5-[3-(2,4-difluoro-benzylamino)-butyl]-pyridin-2-yl}-amide

The title compound was prepared as a white solid, LC-MS M+1=544.9.

### Example 140

### 2-[2-(3,5-Difluoro-phenyl)-acetylamino]-pentanoic Acid {5-[3-(4-chloro-benzylamino)-butyl]-pyridin-2-yl}-amide

The title compound was prepared as a white solid, LC-MS M+1=542.9.

### Example 141

### 2-[2-(3,5-Difluoro-phenyl)-acetylamino]-pentanoic Acid {5-[3-(4-methoxy-benzylamino)-butyl]-pyridin-2-yl}-amide

The title compound was prepared as a white solid, LC-MS M+1=539.5.

### Example 142

### 2-[2-(3,5-Difluoro-phenyl)-acetylamino]-pentanoic Acid {5-[3-(2-fluoro-benzylamino)-butyl]-pyridin-2-yl}-amide

The title compound was prepared as a white solid, LC-MS M+1=527.4.

### Example 143

### 2-[2-(3,5-Difluoro-phenyl)-acetylamino]-pentanoic Acid {5-[3-(3-chloro-benzylamino)-butyl]-pyridin-2-yl}-amide

The title compound was prepared as a white solid, LC-MS M+1=542.9.

### Example 144

### 2-[2-(3,5-Difluoro-phenyl)-acetylamino]-pentanoic Acid {5-[3-(4-fluoro-benzylamino)-butyl]-pyridin-2-yl}-amide

The title compound was prepared as a white solid, LC-MS M+1=527.0.

### Example 145

### 2-[2-(3,5-Difluoro-phenyl)-acetylamino]-pentanoic Acid {5-[3-(3-fluoro-benzylamino)-butyl]-pyridin-2-yl}-amide

The title compound was prepared as a white solid, LC-MS M+1=526.9.

### Example 146

### 2-[2-(3,5-Difluoro-phenyl)-acetylamino]-pentanoic Acid {5-[3-(4-trifluoromethyl-benzylamino)-butyl]-pyridin-2-yl}-amide

The title compound was prepared as a white solid, LC-MS M+1=577.1.

### Example 147

### 2-[2-(3,5-Difluoro-phenyl)-acetylamino]-pentanoic Acid [5-(3-benzylamino-butyl)-pyridin-2-yl]-amide

The title compound was prepared as a white solid, LC-MS M+1=509.0.

### Example 148

### 2-[2-(3,5-Difluoro-phenyl)-acetylamino]-pentanoic Acid [5-(3-benzylamino-butyl)-pyridin-2-yl]-amide

The title compound was prepared as a white solid, LC-MS M+1=509.0.

### Example 149

### 2-[2-(3,5-Difluoro-phenyl)-acetylamino]-pentanoic Acid [5-(3-hydroxy-3-methyl-butyl)-pyridin-2-yl]-amide

To a −78° C. solution of 2-[2-(3,5-difluoro-phenyl)-acetylamino]-pentanoic acid [5-(3-oxo-butyl)-pyridin-2-yl]-amide (51.5 mg, 0.12 mmol) in 1 ml of dry THF was added 1.6 M MeLi in Et2O (0.023 ml, 0.37 mmol) and the resulting mixture was stirred at −78° C. for 20 min and tic showed no startinbg material left. The mixture was quenched with dilute HCl, extracted with ethyl acetate. The organic layer was dried over MgSO4, filtered. The filtrate was concentrated to dryness and the residue was purified by Shimadzu HPLC to give a white solid of the title compound, LC-MS RT=2.2 min, M+1=434.0.

### Example 150

### 2-[2-(3,5-Difluoro-phenyl)-acetylamino]-pentanoic Acid (5-formyl-pyridin-2-yl)-amide.

2-[2-(3,5-Difluoro-phenyl)-acetylamino]-pentanoic Acid (5-hydroxymethyl-pyridin-2-yl)-amide (364 mg, 0.97 mmol) and Dess-Martin Reagent (500 mg, 1.2 mmol) were stirred in dichloromethane/dimethyl sulfoxide at room temperature for 90 minutes. The mixture was partially concentrated, quenched with water and ethyl acetate, and filtered. The organic layer was extracted and concentrated to give the title compound as a white solid. APCI-MS [M+l]=376.1.

### Example 151

### 2-[2-(3,5-Difluoro-phenyl)-acetylamino]-pentanoic Acid {5-[1-(4-methyl-piperidin-1-yl)-ethyl]-pyridin-2-yl}-amide

2-[2-(3,5-Difluoro-phenyl)-acetylamino]-pentanoic acid (5-acetyl-pyridin-2-yl)-amide (100 mg, 0.26 mmol), 4-methyl-piperidine (51 mg, 0.51 mmol), sodium triacetoxyborohydride (108 mg, 0.51 mmol), and acetic acid (15 mg, 0.26 mmol) were stirred in 1,2-dichloroethane at room temperature for 3 hours. An additional 0.2 mL of 4-methyl-piperidine and 50 mL of acetic acid were added and the mixture was stirred at 45° C. for 1 hour. An additional 50 mg of sodium triacetoxyborohydride was then added and the mixture was stirred at 45° C. overnight. The mixture was quenched with dilute sodium hydroxide and extracted with dichloromethane. The organic layer was concentrated and purified by biotage column to give the title compound as a beige glass form. APCI-MS [M+1]=473.2.

### Example 152

### 2-[2-(3,5-Difluoro-phenyl)-acetylamino]-pentanoic Acid (5-ylnyl-pyrazin-2-yl)-amide

2-[2-(3,5-Difluoro-phenyl)-acetylamino]-pentanoic acid (5-bromo-pyrazin-2-yl)-amide (150 mg, 0.35 mmol), tributyl-ylnyl-stannane (134 mg, 0.42 mmol), and trans-dichlorobis-triphenylphosphane-palladium (II) (12 mg, 0.018 mmol) were stirred in toluene heated to reflux for 3 hours. The mixture was quenched with water and extracted with ethyl acetate. The organic layer was concentrated and purified by biotage column to give the title compound as a white solid. LC-MS [M+1]=374.9.

### Example 153

### 2-[2-(3,5-Difluoro-phenyl)-acetylamino]-pentanoic Acid {5-[1-(4-phenyl-Piperidin-1-yl)-ethyl]-pyridin-2-yl}-amide

2-[2-(3,5-Difluoro-phenyl)-acetylamino]-pentanoic acid (5-acetyl-pyridin-2-yl)-amide (200 mg, 0.51 mmol), 4-phenyl-piperidine (166 mg, 1.0 mmol), acetic acid (31 mg, 0.51 mmol), and sodium sulfate (500 mg) were stirred in 1,2-dichloroethane heated to 45° C. for 3 hours. Sodium triacetoxyborohydride (217 mg, 1.0 mmol) was then added to the mixture and left to stir overnight at 45° C. The mixture was quenched with dilute sodium hydroxide and extracted with dichloromethane. The organic layer was concentrated and purified by biotage column to give the title compound as a white solid. APCI-MS [M+1]=535.4.

### Example 154

### 2-[2-(3,5-Difluoro-phenyl)-acetylamino]-pentanoic Acid (5-formyl-pyrazin-2-yl)-amide

2-[2-(3,5-Difluoro-phenyl)-acetylamino]-pentanoic acid (5-ylnyl-pyrazin-2-yl)-amide (500 mg, 1.3 mmol) was dissolved in dichloromethane and cooled in a dry acetone bath. Ozone was bubbled into the mixture for 20 minutes. Nitrogen was then bubbled through the mixture for 15 minutes. 3 mL of dimethyl sulfide was added and the mixture was warmed to room temperature, stirred for 30 minutes, and concentrated. The residue was triturated with dichloromethane and filtered to give the title compound as a beige powder. LC-MS [M+1]=377.0, RT=2.2 min.

Using the procedures described herein, Examples 155-157 are prepared.

### Example 155

### 2-[2-(3-Trifluoromethyl-phenyl)-acetylamino]-pentanoic Acid [5-(3-oxo-butyl)-pyridin-2-yl]-amide.

### Example 156

### 2-[2-(3-Trifluoromethyl-phenyl)-acetylamino]-pentanoic Acid [5-(1-methyl-3-oxo-butyl)-pyridin-2-yl]-amide

### Example 157

### 2-[2-(3-Trifluoromethoxy-phenyl)-acetylamino]-pentanoic Acid [5-(1-methyl-3-oxo-butyl)-pyridin-2-yl]-amide

### Example 158

### 2-[2-(3,5-Difluoro-phenyl)-acetylamino]-pentanoic Acid (5-butylaminomethyl-pyrazin-2-yl)-amide

2-[2-(3,5-Difluoro-phenyl)-acetylamino]-pentanoic acid (5-formyl-pyrazin-2-yl)-amide (80 mg, 0.21 mmol), butylamine (222 mg, 3.0 mmol), acetic acid (42 mg, 0.70 mmol), sodium sulfate (500 mg), and sodium cyanoborohydride (80 mg, 1.3 mmol) were stirred in dichloromethane heated to 45° C. overnight. The mixture was quenched with dilute sodium hydroxide and extracted with dichloromethane. The organic layer was concentrated and purified by preparative HPLC to give the title compound as a white glass form. LC-MS [M+1]=434.1, RT=1.8 min.

The following Examples 160-162 were prepared by the method analogous to that described in Example 159 starting from 2-[2-(3,5-difluoro-phenyl)-acetylamino]-pentanoic acid (5-formyl-pyrazin-2-yl)-amide and an appropriate amine and a reducing agent.

### Example 160

### 2-[2-(3,5-Difluoro-phenyl)-acetylamino]-pentanoic Acid {5-[(3,3-dimethyl-butylamino)-methyl]-pyrazin-2-yl}-amide

The title compound was prepared by reacting 2-[2-(3,5-difluoro-phenyl)-acetylamino]-pentanoic acid (5-formyl-pyrazin-2-yl)-amide, 3,3-dimethyl-butylamine, acetic acid, sodium sulfate, and sodium cyanoborohydride. The title compound was obtained as a white solid. LC-MS [M+1]=462.3, RT=1.7 min.

### Example 161

### 2-[2-(3,5-Difluoro-phenyl)-acetylamino]-pentanoic Acid {5-[(1-phenyl-propylamino)-methyl]-pyrazin-2-yl}-amide

The title compound was prepared by reacting 2-[2-(3,5-difluoro-phenyl)-acetylamino]-pentanoic acid (5-formyl-pyrazin-2-yl)-amide, 1-phenyl-propylamine, acetic acid, sodium sulfate, and sodium cyanoborohydride. The title compound was obtained as a white glass form. LC-MS [M+1]=496.0, RT=1.9 min.

### Example 162

### 2-[2-(3,5-Difluoro-phenyl)-acetylamino]-pentanoic Acid [5-(4-methyl-piperazin-1-ylmethyl)-pyrazin-2-yl]-amide

The title compound was prepared by reacting 2-[2-(3,5-Difluoro-phenyl)-acetylamino]-pentanoic acid (5-formyl-pyrazin-2-yl)-amide, 1-methyl-piperazine, acetic acid, sodium sulfate, and sodium cyanoborohydride. The title compound was obtained as a white glass form. LC-MS [M+1]=461.1, RT=1.4 min.

### Example 163

### 2-[2-(3,5-Difluoro-phenyl)-acetylamino]-pentanoic Acid {5-[(1-benzyl-pyrrolidin-3-ylamino)-methyl]-pyrazin-2-yl}-amide

2-[2-(3,5-Difluoro-phenyl)-acetylamino]-pentanoic acid (5-formyl-pyrazin-2-yl)-amide (100 mg, 0.26 mmol), 1-benzyl-pyrrolidin-3-ylamine (94 mg, 0.53 mmol), acetic acid (16 mg, 0.26 mmol), and sodium sulfate (500 mg) were stirred in dichloromethane heated to 45° C. for 1 hour. Sodium triacetoxyborohydride (112 mg, 0.53 mmol) was added to the mixture and stirred at 45° C. overnight. The mixture was quenched with dilute sodium hydroxide and extracted with dichloromethane. The organic layer was concentrated and purified by preparative HPLC to give the title compound as a beige glass form. LC-MS [M+1]=537.0, RT=1.6 min.

The following Exemples 164-167 were prepared by the method analogous to that described in the Example 163.

### Example 164

### 2-[2-(3,5-Difluoro-phenyl)-acetylamino]-pentanoic Acid [5-(isobutylamino-methyl)-pyrazin-2-yl]-amide

The title compound was prepared by reacting 2-[2-(3,5-Difluoro-phenyl)-acetylamino]-pentanoic acid (5-formyl-pyrazin-2-yl)-amide, isobutylamine, acetic acid, sodium sulfate, and sodium triacetoxyborohydride. The title compound was obtained as a white solid. LC-MS [M+1]=434.0, RT=1.6 min.

### Example 165

### 2-[2-(3,5-Difluoro-phenyl)-acetylamino]-pentanoic Acid [5-(benzylamino-methyl)-pyrazin-2-yl]-amide

The title compound was prepared by reacting 2-[2-(3,5-Difluoro-phenyl)-acetylamino]-pentanoic acid (5-formyl-pyrazin-2-yl)-amide, benzylamine, acetic acid, sodium sulfate, and sodium triacetoxyborohydride. The title compound was obtained as a yellow oil. LC-MS [M+1]=468.0, RT=1.6 min.

### Example 166

### 2-[2-(3,5-Difluoro-phenyl)-acetylamino]-pentanoic Acid {5-[(3-methyl-butylamino)-methyl]-pyrazin-2-yl}-amide

The title compound was prepared by reacting 2-[2-(3,5-Difluoro-phenyl)-acetylamino]-pentanoic acid (5-formyl-pyrazin-2-yl)-amide, 3-methyl-butylamine, acetic acid, sodium sulfate, and sodium triacetoxyborohydride. The title compound was obtained as a white solid. LC-MS [M+1]=448.1, RT=2.0 min.

### Example 167

### 2-[2-(3,5-Difluoro-phenyl)-acetylamino]-pentanoic Acid [5-phenethylamino-methyl)-pyrazin-2-yl]-amide

The title compound was prepared by reacting 2-[2-(3,5-Difluoro-phenyl)-acetylamino]-pentanoic acid (5-formyl-pyrazin-2-yl)-amide, phenethylamine, acetic acid, sodium sulfate, and sodium triacetoxyborohydride. The title compound was obtained as a yellow oil. LC-MS [M+1]=482.0, RT=1.8 min.

### Example 168

### 2-[2-(3,5-Difluoro-phenyl)-acetylamino]-pentanoic Acid 15-(1-hydroxy-ethyl)-pyrazin-2-yl]-amide

To a −78° C. solution of 2-[2-(3,5-difluoro-phenyl)-acetylamino]-pentanoic acid (5-formyl-pyrazin-2-yl)-amide (100 mg, 0.26 mmol) in tetrahydrofuran in a dry ice/acetone bath was added dropwise methyllithium (0.66 mL of a 1.6 M solution in ether, 1.1 mmol) and the mixture was allowed to warm to room temperature and stir overnight. The mixture was quenched with dilute hydrochloric acid and extracted with ethyl acetate. The organic layer was concentrated and purified by preparative HPLC to give the title compound as a beige glass form. LC-MS [M+1]=393.0, RT=1.9 min.

### Example 169

### 2-[2-(3,5-Difluoro-phenyl)-acetylamino]-pentanoic Acid 15-(1-hydroxy-pentyl)-pyrazin-2-yl]-amide

To a −78° C. solution of 2-[2-(3,5-difluoro-phenyl)-acetylamino]-pentanoic acid (5-formyl-pyrazin-2-yl)-amide in tetrahydrofuran in dry ice/acetone bath was added an excess (about 5 eq) of butylyllithium (1.6 M solution in ether) at −78° C. and the mixture was allowed to warm to room temperature and stir overnight. The mixture was quenched with dilute hydrochloric acid and extracted with ethyl acetate. The organic layer was concentrated and purified by preparative HPLC to give the title compound as a beige glass form. LC-MS [M+1]=435.0, RT=2.6 min.

### Example 170

### 2-[2-(3,5-Difluoro-phenyl)-acetylamino]-pentanoic Acid (5-hydroxymethyl-pyrazin-2-yl)-amide

A mixture of 2-[2-(3,5-difluoro-phenyl)-acetylamino]-pentanoic acid (5-formyl-pyrazin-2-yl)-amide and 5 eq of sodium borohydride in methanol was stirred at rt for 10 min. The mixture was quenched with water and extracted with ethyl acetate. The organic layer was concentrated to give the title compound as a white solid. LC-MS [M+1]=379.0, RT=2.0 min.

### Example 171

### 2-[2-(3,5-Difluoro-phenyl)-acetylamino]-pentanoic Acid {5-[(4-chloro-phenyl)-hydroxy-methyl]-pyrazin-2-yl}-amide

2-[2-(3,5-Difluoro-phenyl)-acetylamino]-pentanoic acid (5-formyl-pyrazin-2-yl)-amide (100 mg, 0.26 mmol) was dissolved in tetrahydrofuran and cooled in a dry acetone bath. 4-Chloro-phenylmagnesiumbromide (1.1 mL of a 1.0 M solution in ether, 1.1 mmol) was added dropwise and the mixture was allowed to warm to room temperature and stir overnight. The mixture was quenched with dilute hydrochloric acid and extracted with dichloromethane. The organic layer was concentrated and purified by preparative HPLC to give the title compound as a yellow oil. LC-MS [M+1]=488.9, RT=2.6 min.

### Example 172

### 6-[2-(6,8-difluoro-1,2,3,4-tetrahydro-naphthalen-2-ylamino)-pentanoylamino]-nicotinic Acid methyl ester

6,8-Difluoro-3,4-dihydro-1H-naphthalen-2-one (634 mg, 3.5 mmol), 6-(2-amino-pentanoylamino)-nicotinic acid methyl ester (1.0 g, 3.5 mmol), acetic acid (836 mg, 13.9 mmol), and sodium sulfate (1.0 g) were stirred in dichloromethane heated to 45° C. for 3 hours. Sodium triacetoxyborohydride (1.47 g 7.0 mmol) was added to the reaction and the mixture was left to stir overnight at 45° C. Sodium cyanoborohydride (650 mg, 10.3 mmol) was then added to the solution and stirred for three days at room temperature. The mixture was quenched with dilute sodium hydroxide and extracted with dichloromethane. The organic layer was concentrated and purified by preparative HPLC to give the title compound as a white solid. LC-MS [M+1]=418.0 at RT=2.0 min.

### Example 173

### 2-[2-(3,5-difluoro-phenyl)-acetylamino]-pentanoic Acid [5-(1-hydroxy-1-methyl-allyl)-pyridin-2-yl]-amide

2-[2-(3,5-Difluoro-phenyl)-acetylamino]-pentanoic acid (5-acetyl-pyridin-2-yl)-amide (100 mg, 0.26 mmol) was dissolved in tetrahydrofuran and cooled in a dry acetone bath. Vinylmagnesium bromide (1.0 mL of a 1.0 M solution in tetrahydrofuran, 1.0 mmol) was added dropwise and the mixture was allowed to warm to room temperature overnight. The mixture was quenched with dilute hydrochloric acid and extracted with ethyl acetate. The organic layer was concentrated and purified by biotage column to give the title compound as a clear oil. LC-MS [M+1]=418.0 at RT=2.3 min.

### Example 174

### 2-[2-(3,5-difluoro-phenyl)-acetylamino]-pentanoic Acid {5-[1-(4-methyl-piperazin-1-yl)-ethyl]-pyridin-2-yl}-amide

2-[2-(3,5-Difluoro-phenyl)-acetylamino]-pentanoic acid (5-acetyl-pyridin-2-yl)-amide (200 mg, 0.51 mmol), 1-methyl-piperazine (114 μL, 1.0 mmol), acetic acid (31 mg, 0.51 mmol), and sodium sulfate (200 mg) were stirred in dichloromethane heated to 45° C. for 2 hours. Sodium triacetoxyborohydride (200 mg, 0.95 mmol) was added and the mixture was left to stir at 45° C. overnight. The mixture was quenched with dilute sodium hydroxide and extracted with dichloromethane. The organic layer was concentrated and purified by preparative HPLC to give the title compound as a beige glass form. LC-MS [M+1]=474.0 at RT=1.7 min.

### Example 175

### 2-[2-(3,5-difluoro-phenyl)-acetylamino]-pentanoic Acid (5-{[methyl-(3-methyl-butyl)-amino]-methyl}-pyrazin-2-yl)-amide

The title compound was prepared by the method described in preparation (Kevin-3 new) by reacting 2-[2-(3,5-difluoro-phenyl)-acetylamino]-pentanoic acid (5-formyl-pyrazin-2-yl)-amide, methyl-(3-methyl-butyl)-amine, acetic acid, sodium sulfate, and sodium triacetoxyborohydride. The title compound was obtained as a white glass form. LC-MS [M+1]=462.0 at RT=1.8 min.

### Example 176

### 2-[2-(3,5-Difluoro-phenyl)-acetylamino]-pentanoic Acid {5-[(3-hydroxy-butylamino)-methyl]-pyrazin-2-yl}-amide

The title compound was prepared by the method described in preparation (Kevin-3 new) by reacting 2-[2-(3,5-difluoro-phenyl)-acetylamino]-pentanoic acid (5-formyl-pyrazin-2-yl)-amide, 4-amino-butan-2-ol, acetic acid, sodium sulfate, and sodium triacetoxyborohydride. The title compound was obtained as a white glass form. LC-MS [M+1]=450.0 at RT=1.5 min.

### Example 177

### 2-[2-(3,5-difluoro-phenyl)-acetylamino]-pentanoic Acid {5-[(1-phenyl-ethylamino)-methyl-pyrazin-2-yl]-amide

The title compound was prepared by the method described in preparation (Kevin-3 new) by reacting 2-[2-(3,5-difluoro-phenyl)-acetylamino]-pentanoic acid (5-formyl-pyrazin-2-yl)-amide, 1-phenyl-ethylamine, acetic acid, sodium sulfate, and sodium triacetoxyborohydride.

The title compound was obtained as a yellow oil. LC-MS [M+1]=482.0 at RT=1.9 min.

### Example 178

### 2-[2-(3,5-difluoro-phenyl)-acetylamino]-pentanoic Acid (5-acetyl-pyrazin-2-yl)-amide

2-[2-(3,5-Difluoro-phenyl)-acetylamino]-pentanoic acid [5-(1-hydroxy-ethyl)-pyrazin-2-yl]-amide (50 mg, 0.13 mmol) and Dess-Martin's Reagent (65 mg, 0.15 mmol) were stirred in dichloromethane/dimethyl sulfoxide at room temperature overnight. The dichloromethane was evaporated and the mixture was quenched with water and extracted with ethyl acetate. The organic layer was concentrated and purified by preparative HPLC to give the title compound as a white solid. LC-MS [M+1]=391.0 at RT=2.4 min.

### Example 179

### 2-(6,8-Difluoro-1,2,34-tetrahydro-naphthalen-2-ylamino)-pentanoic Acid (5-bromo-pyrazin-2-yl)-amide

6,8-Difluoro-3,4-dihydro-1H-naphthalen-2-one (250 mg, 1.37 mmol), 2-amino-pentanoic acid (5-bromo-pyrazin-2-yl)-amide (375 mg, 1.37 mmol), acetic acid (0.16 ml, 2.75 mmol), and sodium sulfate (0.5 g) were stirred in dichloromethane heated to 45° C. for 2 hours. Sodium triacetoxyborohydride (580 mg, 2.75 mmol) was added to the reaction and the mixture was left to stir overnight at 45° C. The mixture was quenched with dilute sodium hydroxide and extracted with dichloromethane. The organic layer was concentrated and purified by silica gel column chromatography using 5-20% ethyl acetate in hexane as eluent to give the title compound as an off-white solid. LC-MS [M+1]=441 at RT=1.8 min.

### Example 180

### 2-(6,8-Difluoro-1,2,3,4-tetrahydro-naphthalen-2-ylamino)-pentanoic Acid (5-bromo-pyridin-2-yl)-amide trifluoroacetic Acid

6,8-Difluoro-3,4-dihydro-1H-naphthalen-2-one (636 mg, 2.68 mmol), 2-amino-pentanoic acid (5-bromo-pyridin-2-yl)-amide (854 mg, 3.49 mmol), acetic acid (0.3 ml), in dichloromethane (15 ml) and DMF (7.5 ml) was stirred at room temperature for 3 hours. Sodium triacetoxyborohydride (854 mg, 4.03 mmol) was added to the reaction and the mixture was left to stir at room temperature overnight. An additional 854 mg of sodium triacetoxy borohydride and 0.27 ml of acetic acid were added and the reaction was monitored by tic. After stirring at room temperature for 2 hr, sodium cyanoborohydride (87 mg) was added and the resulting mixture was stirred at room temperature for 30 min. The mixture was quenched with dilute sodium hydroxide and extracted with ethyl acetate. The organic layer was dried over MgSO4, filtered, concentrated and purified by prep HPLC to give the title compound as a trifluoroacetic acid salt as an off-white solid. LC-MS [M+1]=438 and 440.2 at RT=2.1 min.

### Example 181

### 2-(6,8-Difluoro-1,2,3,4-tetrahydro-naphthalen-2-ylamino)-pentanoic Acid [5-(3-oxo-butyl)-pyrazin-2-yl]-amide

A mixture of 2-(6,8-difluoro-1,2,3,4-tetrahydro-naphthalen-2-ylamino)-pentanoic acid (5-bromo-pyrazin-2-yl)-amide (315 mg, 0.649 mmol), vinyl methyl ketone (68 mg, 0.974 mmol), tran-dichlorobis(triphenylphosphine)palladium (II) (50 mg, 0.072 mmol), diisopropylethylamine (126 mg, 0.17 ml, 0.974 mmol) in propionitrile (10 ml) was heated at reflux for 4 hr, tehn stirred at room temperature overnight. The mixture was filtered through celite, washed with ethyl acetate and the filtrate was concentrated to dryness. The residue was purified by silica gel column chromatography using 10-75% ethyl acetate in hexane as eluent to isolate the following three components:
2-(6,8-Difluoro-1,2,3,4-tetrahydro-naphthalen-2-ylamino)-pentanoic acid [5-(3-oxo-butyl)-pyrazin-2-yl]-amide, LC-MS, M+1=431 at RT=1.7 min,
   The following compounds were also prepared using the procedure described hereinabove.
2-(6,8-difluoro-1,2,3,4-tetrahydro-naphthalen-2-ylamino)-pentanoic acid pyrazin-2-ylamide, LC-MS M+1=360 at RT=2.2 min,
2-(6,8-difluoro-1,2,3,4-tetrahydro-naphthalen-2-ylamino)-pentanoic acid (5-cyano-pyrazin-2-yl)-amide, LC-MS M+1=386 at RT=1.8 min.

### Example 182

### 2-(5,7-Difluoro-1,2,3,4-tetrahydro-naphthalen-2-ylamino)-pentanoic Acid 15-[3-(1-phenyl-ethylamino)-butyl]-pyridin-2-yl}-amide

6,8-Difluoro-3,4-dihydro-1H-naphthalen-2-one (87.5 mg, 0.48 mmol), 2-amino-pentanoic acid {5-[3-(1-phenyl-ethylamino)-butyl]-pyridin-2-yl}-amide hydrogen chloride (194 mg, 0.48 mmol), acetic acid (0.04 ml), in dichloromethane (5 ml) and DMF (2.5 ml) was stirred at room temperature for 3 hours. Sodium triacetoxyborohydride (153 mg, 0.72 mmol) was added to the reaction and the mixture was left to stir at room temperature overnight. An additional 153 mg of sodium triacetoxy borohydride and 0.04 ml of acetic acid were added and the reaction was monitored by tic. After stirring at room temperature for 2 hr, sodium cyanoborohydride (48 mg) was added and the resulting mixture was stirred at room temperature for 30 min. The mixture was quenched with dilute sodium hydroxide and extracted with ethyl acetate. The organic layer was dried over MgSO4, filtered, concentrated and purified by prep HPLC to give the title compound as a trifluoroacetic acid salt as an off-white solid. LC-MS [M+1]=535.3 and 533.4 at RT=1.64 min.

Intermediate Synthesis

Exemplary Procedures for Intermediates are as Follows;

### Intermediate 1

### t-butyl[1-methyl-2-oxo-2-(pyrazin-2-ylamino)ethyl]carbamate

To a solution of amino-pyrazine (936 mg, 9.8 mmol) in THF (10 ml) and toluene (30 ml) was added 2.0 M AlMe3 in toluene (4.9 ml, 9.8 mmol) at room temperature and the resulting mixture was stirred at room temperature for 2 hr. 2-[(t-butoxycarbonyl)amino]propanoic acid was added to the reaction mixture and stirred overnight. The mixture was quenched with Rochelle salt, the organic layer was separated. The organic layer was washed with dilute HCl to PH 6, brine, separated, dried and concentrated to give the title compound as a white solid (936 mg). ¹H NMR (DMSO-d₆) d9.3(s,1H), 8.4(s,1H), 8.3(s,1H), 7.2(d,1H), 4.2(m,1H), 1.35(s,9H), 1.25(d,3H) ppm. M+1=267.3.

The following intermediates 2—were prepared by the method analogous to that described in Intermediate 1 starting from an appropriate ester with an amine.

### Intermediate 2

### t-butyl[1-methyl-2-oxo-2-(Pyrazin-2-ylamino)propyl]carbamate

The title compound was prepared by the method analogous arting from 2-amino-pyrazine, AlMe3 and 2-tert-butoxycarbonylamino-butyric acid methyl ester. ¹H NMR (DMSO-d₆) d9.3(s,1H), 8.4(s,1H), 8.3(s,1H), 7.1 (d, 1H), 4.1 (m,1H), 1.7(m,1H), 1.5(m,1H), 1.4(s,9H), 0.99(t,3H) ppm. M+1=281.2.

### Intermediate 3

### t-butyl[1-methyl-2-oxo-2-(Pyrazin-2-ylamino)butyl]carbamate

The title compound was prepared by the analogous method starting from 2-amino-pyrazine, AlMe3 and 2-tert-butoxycarbonylamino-pentanoic acid methyl ester. ¹H NMR (CDCl3) d9.5(s,1H), 8.8(brs,1H), 8.35(s,1H), 8.25(s,1H), 4.3(m,1H), 1.9(m,1H), 1,4-1,6(m,12H), 0.97(t,3H) ppm. M+1=295.4.

### Intermediate 4

### [1-(5-Bromo-pyrimidin-2-ylcarbamoyl)-butyl]-carbamic acid tert-butyl ester

LC-MS, RT=2.0 min, M+1=319.2, M-1=373.3; APCI M+1=375.

### Intermediate 5

### [1-(5-Bromo-pyridin-2-ylcarbamoyl)-butyl]-carbamic acid tert-butyl ester

prepared as a white solid after trituating with Et₂O, LC-MS RT=2.51 min, M+1=373.9.

### Intermediate 6

### {1-[5-(1-Methyl-3-oxo-but-1-enyl)-pyridin-2-ylcarbamoyl]-butyl}-carbamic acid tert-butyl ester

A mixture of [1-(5-bromo-pyridin-2-ylcarbamoyl)-butyl]-carbamic acid tert-butyl ester (11.17 g, 30 mmol), 3-pentene-2-one (5.41 g, 45 mmol), diisopropylethylamine (5.97 g, 46.2 mmol), palladium acetate (741 mg, 3.3 mmol), tri-o-tolylphosphine (1.64 g, 5.4 mmol) in propionitrile (250 ml) was heated at reflux for 3 hrs. The mixture cooled to rt, diluted with ethyl acetate and water. The organic layer was separated, dried over MgSO4, filtered, and concentrated to dryness to give 5.1 g of crude product as a brown oil. The brown oil was purified by silica gel column chromatography using Hexane:EtOAc=4:1 to 7:3 as eluent to give 3.24 g of {1-[5-(1-Methyl-3-oxo-but-1-enyl)-pyridin-2-ylcarbamoyl]-butyl}-carbamic acid tert-butyl ester as a yellow solid, LC-MS, RT=2.5 min, M+1=376.0 and 3.52 g of {1-[5-(1-Methyl-3-oxo-butyl)-pyridin-2-ylcarbamoyl]-butyl}-carbamic acid tert-butyl esteras a yellow solid, LC-MS, RT=2.4 min, M+1=378.0.

### Intermediate 7

### {1-[5-(3-Oxo-but-1-enyl)-pyridin-2-ylcarbamoyl]1-butyl}-carbamic Acid tert-butyl Ester

The title compound was prepared by the method analogous to that described in Intermediate 6, RT=2.3 min, M+1=362.0.

### Intermediate 8

### {1-[5-(1-Methyl-3-oxo-butyl)-pyridin-2-ylcarbamoyl]-butyl}-carbamic acid tert-butyl ester

A mixture of {1-[5-(1-Methyl-3-oxo-but-1-enyl)-pyridin-2-ylcarbamoyl]-butyl}-carbamic acid tert-butyl ester (3.16 g, 8.41 mmol) and PtO2 (632 mg) in ethyl acetate was hydrogenated at 50 psi overnight. TLC showed some starting material left. Additional 316 mg of PtO2 was added and the mixture was hydrogenerated for additional 2 hr. No starting material left. The mixture was filtered through celite. The filtrate was concentrated to dryness. The residue was purified by column chromatography using Hexane:ETOAc=1:1 as eluent to give 1.97 g of the title compound as a white glass foram, LC-MS RT=2.1 min, M+1=378.2.

### Intermediate 9

### {1-[5-(3-Oxo-butyl)-pyridin-2-ylcarbamoyl]-butyl}-carbamic acid tert-butyl ester.

### Intermediate 10

### {1-[5-(3-Hydroxy-butyl)-pyridin-2-ylcarbamoyl-1-butyl]-carbamic acid tert-butyl ester

A mixture of {1-[5-(3-oxo-but-1-enyl)-pyridin-2-ylcarbamoyl]-butyl}-carbamic acid tert-butyl ester (728 mg, 2.0 mmol) and PtO2 (145 mg) in ethyl acetate was hydrogenated at 50 psi for 3 hr. TLC showed no starting material left. The mixture was filtered through celite. The filtrate was concentrated to dryness. The residue was purified by column chromatography using Hexane:ETOAc=1:1 as eluent to give 345 mg of {1-[5-(3-Oxo-butyl)-pyridin-2-ylcarbamoyl]-butyl}-carbamic acid tert-butyl ester as a white solid, LC-MS RT=2.2 min, M+1=364.1 and 171 mg of the over reduced {1-[5-(3-Hydroxy-butyl)-pyridin-2-ylcarbamoyl]-butyl}-carbamic acid tert-butyl ester as awaxy white solid, LC-MS RT=2.0 min, M+1=366.1.

### Intermediate 11

### 6-(2-tert-Butoxycarbonylamino-pentanoylamino)-nicotinic Acid Methyl Ester

A mixture of 2-amino-nicotinic acid methyl ester (30 g, 197 mmol) and 2-tert-butoxycarbonylamino-pentanoic acid (42.8 g, 197 mmol), HBOT (28 g, 207 mmol), EDC. HCl (45.2 g, 236.8 mmol), triethylamine (79.7 g, 110 ml, 789 mmol) in methylene chloride was stirred at room temperature over the weekend. The mixture was quenched with water, extracted with methylene chloride. The organic layer was washed with dilute HCl, separated, dried over anhydrous Na2SO4, filtrered, and concentrated to give 50.5 g of an off-white solid. APCI M+1=352.

### Intermediate 12

### 2-Amino-N-pyrazin-2-yl-propionamide hydrochloride

A mixture of t-butyl[1-methyl-2-oxo-2-(pyrazin-2-ylamino)ethyl]carbamate (290 mg) and 4 M HCl(g) in dioxane was stirred at room temperature for 90 min. The mixture was concentrated to dryness under reduced pressure to give the title compound as a white solid. ¹H NMR (DMSO-d₆) d9.3(s,1H), 8,3-8,5(m,4H), 4.1(m,1H), 1.4(d,3H) ppm. M+1=167.2.

### Intermediate 13

### 2-amino-N-pyrazin-2-ylbutyramide hydrochloride

The title compound was prepared by reacting t-butyl[1-methyl-2-oxo-2-(pyrazin-2-ylamino)propyl]carbamate and HCl in dioxane as described above.

¹H NMR (DMSO-d₆) d9.3(s,1H), 8.3-8.5(m,5H), 4.1 (m,1H), 1.86(m,2H), 0.93(t,3H) ppm. M+1=181.2.

### Intermediate 14

### 2-Amino-pentanoic Acid pyrazin-2-ylamide hydrochloride

The title compound was prepared by reacting t-butyl[1-methyl-2-oxo-2-(pyrazin-2-ylamino)butyl]carbamate and HCl in dioxane as described above.

¹H NMR (DMSO-d₆) d9.3(s,1H), 8.3-8.6(m,4H), 4.1(m,1H), 1.8(m,1H), 1.2-1.7(m,3H), 0.87(t,3H) ppm. M+1=195.3.

### Intermediate 15

### 2-Amino-pentanoic Acid [5-(1-methyl-3-oxo-butyl)-pyridin-2-yl]-amide

prepared as a white solid, LC-MS, RT=1.0 min, M+1=278.3.

### Intermediate 16

### 2-Amino-pentanoic Acid 15-(3-oxo-butyl)-pyridin-2-yl]-amide

prepared as an off-white solid, APCI M+1=264.3.

### Intermediate 17

### 3-(2-Amino-pyridin-5-yl)-but-2-enoic Acid Methyl Ester

A mixture of 2-amino-5-bromo-pyridine (2.5 g, 14.45 mmol), methyl crotonate (1.73 g, 17.34 mmol), triethylamine (3.21 g, 17.34 mmol), palladium acetate (129.7 mg, 5.8 mmol), tri-o-tolylphosphine (351 mg, 1.16 mmol) in N-methylpyrrolidine (25 ml) was heated at reflux overnight. An additional palladium acetate (65 mg) and tri-tolyl phosphine (176 mg) was added and the mixture was heated overnight. The mixture cooled to rt, diluted with ethyl acetate and water. The organic layer was separated, dried over MgSO4, filtered, and concentrated to dryness to give 5.1 g of crude product as a brown solid. The brown solid was triturated with hexane to give the title compound (1.17 g).

### Intermediate 18

### 3-(2-Amino-pyrimidin-5-yl)-but-2-enoic Acid Methyl Ester

A mixture of 2-amino-5-bromo-pyrimidine (174 mg, 1.0 mmol), methyl crotonate (129 mg, 1.5 mmol), diisopropylethylamine (0.27 ml, 1.54 mmol), palladium acetate (24.7 mg, 0.11 mmol), tri-o-tolylphosphine (54.8 mg, 0.18 mmol) in propionitrile (10 ml) was heated at reflux overnight. The mixture cooled to rt, diluted with ethyl acetate and water. The organic layer was separated, dried over MgSO4, filtered, and concentrated to dryness. The residue was purified by silica gel column chromatography using 1% MeOH in methylene chloride as eluent to give the title compound as a white solid.

### Intermediate 19

### 6-(2-Amino-pentanoylamino)-nicotinic Acid Methyl Ester

To a solution of 6-(2-tert-butoxycarbonylamino-pentanoylamino)-nicotinic acid methyl ester (50.5 g, 143.87 mmol) in 100 ml dioxane was added 4 M HCl in dioxane (216 ml, 863 mmol) at room temperature. The resulting mixture was stirred for 1 hr, then concentrated to dryness, triturated with acetone to give 35.0 g of the title compound as a white solid. APCI M+1=252.

The activity of compounds of Formula I of the present invention in inhibiting gamma-secretase activity was determined in a solubilized membrane preparation generally according to the description provided in McLendon, et al., “Cell-free assays for γ-secretase activity”, FASEB Journal (Vol.14, December 2000, pages 2383-2386), the contents of which are incorporated by reference. Using such assay, the IC₅₀ of the compounds of the present invention for inhibiting gamma-secretase activity were determined. Of those tested, approximately 60% exhibited IC₅₀ values of less than 150 nm, of which greater than about 90% exhibited IC₅₀ values ranging from about 0.88 to about 100 nm, and about 60% thereof exhibited IC₅₀ values less than 40 nm and approximately 50% of those exhibited IC₅₀ values of less than 40 nm. A preferred group of compounds exhibited IC₅₀ values of less than 20 nm, It is to be noted that a significant number of compounds tested exhibited IC₅₀ values of less than 10 nm.

The above preferred embodiments and examples were given to illustrate the scope and spirit of the present invention. These embodiments and examples will make apparent to those skilled in the art other embodiments and examples. These other embodiments and examples are within the contemplation of the present invention. Therefore, the present invention should only be limited by the appended claims.

1. A compound of the Formula 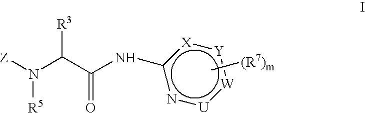
wherein Z is selected from —C(═O)CHR¹R², —C(═S)CHR¹R², —(C═NR⁸)CHR¹R², —C(═O)C(═O)R¹, —SO₂—R¹ and R¹;
m is an integer independently selected from zero, 1, 2, and 3;
R¹ is selected from —C₁-C₂₀ alkyl, —C₂-C₂₀ alkenyl, —C₂-C₂₀ alkynyl, —C₁-C₂₀ alkoxy, —C₂-C₂₀ alkenoxy, —C₂-C₂₀ alkynoxy, —C₃-C₂₀ cycloalkyl, —C₄-C₂₀ cycloalkenyl, -(4-20 membered) heterocycloalkyl, —C₆-C₂₀ aryl and -(5-20 membered)heteroaryl;
wherein R¹ is optionally independently substituted with from one to six fluorine atoms or with from one to three substituents independently selected from the group R^{1a};
R^{1a} is in each instance independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —Cl, —Br, —I, —CN, —NO₂, —NR⁹R¹⁰, —C(═O)NR⁹R¹⁰, —SO₂—NR⁹R¹⁰, —C(═O)R¹¹, —SO₂—R¹¹, —C(═O)OR², —C₃-C₁₅ cycloalkyl, —C₄-C₁₅ cycloalkenyl, -(4-20 membered)heterocycloalkyl, —C₆-C₁₅ aryl, -(5-15 membered)heteroaryl, —C₆-C₁₅ aryloxy and -(5-15 membered)heteroaryloxy, wherein said cycloalkyl, cycloalkenyl, heterocycloalkyl, aryl, heteroaryl, aryloxy and heteroaryloxy are each optionally independently substituted with from one to three substituents independently selected from the group R^{1b};
R^{1b} is in each instance independently selected from —OH, —C₁-C₆ alkyl, —C₂-C₆ alkenyl, —C₂-C₆ alkynyl, —C₁-C₆ alkoxy, —C₂-C₆ alkenoxy, —C₂-C₆ alkynoxy, —C₁-C₆ hydroxyalkyl, —F, —Cl, —Br, —I, —CN, —NO₂, —NR⁹R¹⁰, —C(═O)NR⁹R¹⁰, —C(═O)R¹¹, —SO₂—R¹¹, —C₆-C₁₅ aryloxy and -(5-15 membered)heteroaryloxy, wherein said alkyl, alkenyl and alkynyl aryloxy or heteroaryloxy are each optionally independently substituted with from one to six fluorine atoms or with from one to two substituents independently selected from —C₁-C₄ alkoxy, or with a hydroxy group;
R⁹ and R¹⁰ are in each instance each independently selected from —H, —C₁-C₁₂ alkyl, —C₂-C₁₂ alkenyl, —C₂-C₁₂ alkynyl, CF₃, —C(═O)R¹¹, —SO₂—R¹¹, —C(═O)OR², —C(═O)NR¹¹R¹², —SO₂—NR¹¹R², —(C_{zero}-C₄ alkylene)-(C₃-C₂₀ cycloalkyl), —(C_{zero}-C₄ alkylene)-(C₄-C₈ cycloalkenyl), —(C_{zero}-C₄ alkylene)-((5-10 membered)heterocycloalkyl), (C_{zero}-C₄ alkylene)-(C₆-C₁₀ aryl) and —(C_{zero}-C₄ alkylene)-((5-10 membered)heteroaryl), wherein said alkyl, alkenyl and alkynyl are each optionally independently substituted with from one to six fluorine atoms or with from one to two substituents independently selected from —C₁-C₄ alkoxy, or with a hydroxy group or C(═O)OR¹², and wherein said cycloalkyl, cycloalkenyl, heterocycloalkyl, aryl and heteroaryl are each optionally independently substituted with from one to three substituents independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —C₁-C₆ hydroxyalkyl, —F, —Cl, —Br, —I, —CN, —NO₂, —CF₃, —NH₂, —C(═O)NH₂, —SO₂—NH₂, —C(═O)H and —C(═O)OH, wherein said alkyl, alkenyl and alkynyl substituents are each optionally independently further substituted with from one to six fluorine atoms or with from one to two substituents independently selected from —C₁-C₄ alkoxy, or with a hydroxy group or aryl group;
or NR⁹R¹⁰ may in each instance independently optionally form a heterocycloalkyl moiety of from four to ten ring members, said heterocycloalkyl moiety optionally containing one to two further heteroatoms independently selected from N, O and S, and optionally containing from one to three double bonds, wherein the carbon atoms of the heterocycloalkyl moiety of NR⁹R¹⁰ are optionally independently substituted with from one to three substituents independently selected from —OH, —C₁-C₁₂ alkyl, —C₂-C₁₂ alkenyl, —C₂-C₁₂ alkynyl, —C₁-C₆ alkoxy, —C₂-C₆ alkenoxy, —C₂-C₆ alkynoxy, —F, —Cl, —Br, —I, —CF₃, —NH₂, —C(═O)NH₂, —SO₂—NH₂, —C(═O)R¹¹, SO₂—R¹¹, (C_{zero}-C₄ alkylene)-(C₆-C₁₀ aryl), (C_{zero}-C₄ alkylene)-((5-10 membered heteroaryl), (C_{zero}-C₄ alkylene)-(C₆-C₁₀ cycloalkyl) and (C_{zero}-C₄ alkylene)-((5-10 membered)heterocycloakyl), and wherein the (C_{zero}-C₄ alkylene)-((5-10 membered)heterocycloalkyl) substituent and the nitrogen atoms of said heterocycloalkyl moiety of NR⁹R¹⁰ are each optionally independently substituted with one substituent independently selected from —C₁-C₁₂ alkyl, —C₂-C₁₂ alkenyl, —C₂-C₁₂ alkynyl, —C(═O)NH₂, —SO₂—NH₂, C(═O)R¹¹, SO₂—R¹¹, (C_{zero}-C₄ alkylene)-(C₆-C₁₀ aryl), (C_{zero}-C₄ alkylene)-((5-10 membered)heteroaryl), (C_{zero}-C₄ alkylene)-(C₆-C₁₀ cycloalkyl) and (C_{zero}-C₄ alkylene)-((5-10 membered)heterocycloalkyl), and wherein said alkyl, alkenyl and alkynyl substituents are each optionally independently further substituted with from one to six fluorine atoms, or with from one to two substituents independently selected from —C₁-C₄ alkoxy, or with a hydroxy group;
R¹¹ and R¹² are in each instance each independently selected from hydrogen, —C₁-C₁₅ alkyl, —C₂-C₆ alkenyl, —C₂-C₆ alkynyl, —(C_{zero}-C₄ alkylene)-(C₃-C₁₅ cycloalkyl), —(C_{zero}-C₄ alkylene)-(C₄-C₈ cycloalkenyl), —(C_{zero}-C₄ alkylene)-(C₆-C₁₅ aryl), —(C_{zero}-C₄ alkylene)-((5-15 membered) heterocycloalkyl) and C_{zero}-C₄ alkylene)-((5-15 membered)heteroaryl);
wherein R¹¹ and R¹² are each optionally independently substituted with from one to three substituents independently selected from the group R^{1b};
R² is selected from —H, —OH, —NH₂, —CH₂OH, —OC(═O)CH₃, —C(CH₃)₂OH, —C(CH₃)(CH₂CH₃)(OH), —C(OH)(C_{zero}-C₄ alkyl)(C_{zero-C}₄ alkyl), —OC(═O)R⁴ and —OC(═O)OR⁴, wherein said —OC(═O)R⁴ and —OC(═O)OR⁴ may optionally be a prodrug of the corresponding OH of R²;
R⁴ is selected from —C₁-C₄ alkyl, —CH(OH)(C₁-C₄ alkyl), —CH(OH)(C₅-C₆ aryl), —CH(OH)((5-6 membered)heteroaryl), —CH(OH)(C₅-C₆ cycloalkyl) and —CH(OH)((5-6 membered) heterocycloalkyl);
R³ is selected from —C₁-C₆ alkyl, —C₂-C₆ alkenyl, —C₂-C₆ alkynyl and C_{zero}-C₄ alkylene)-(C₃-C₆ cycloalkyl), wherein when R³ is alkyl, alkenyl or alkynyl, R³ is optionally independently substituted with a substituent independently selected from —C₁-C₄ alkoxy, —F, —OH and —S(C₁-C₄ alkyl);
R⁵ is selected from —H, —C₁-C₄ alkyl, —C₂-C₄ alkenyl, —C₂-C₄ alkynyl, —C(═O)(C₁-C₄ alkyl), —C₆-C₁₀ aryl, -((5-20 membered)heteroaryl), —SO₂—(C₆-C₁₀ aryl), —SO₂-((5-20 membered) heteroaryl), —SO₂—CH₂—(C₆-C₂₀ aryl) and —SO₂—CH₂-((5-20 membered)heteroaryl);
X, Y, W and U are each independently selected from carbon and nitrogen, with the proviso that the 6-membered heteroaryl ring of Formula I that contains X, Y, W and U may not contain more than three nitrogen atoms in the ring, and with the further proviso that no more than two nitrogen atoms in the heteroaryl ring of Formula I that contains X, Y, W and U may be directly adjacent to each other in the ring, and with the even further proviso when R⁷ is —OH and R⁷ is attached to a carbon atom of the heteroaryl ring Formula I that contains X, Y, W and U, and m is 1, 2 or 3, then the —OH group of R⁷ that is attached to a carbon atom of the heteroaryl ring of Formula I may be tautomerizable to a C═O group;
R⁷ is selected from —H, —C₁-C₂₀ alkyl, —C₂-C₂₀ alkenyl, —C₂-C₂₀ alkynyl, —C₁-C₂₀ alkoxy, —C₂-C₂₀ alkenoxy, —C₂-C₂₀ alkynoxy, —F, —Cl, —Br, —I, —CN, —NO₂, —OH, —CF₃, —NR⁹R¹⁰, C₆-C₁₈ aryloxy, —(C₁-C₁₁ alkylene)-NR⁹R¹⁰, —C(═O)NR⁹R¹⁰, —C(═O)R¹¹, —CHO, —SO₂—R¹¹, —C(═O)OR¹², COOH, —C_{zero}-C₄ alkylene)-(C₃-C₂₀ cycloalkyl), —(C_{zero}-C₄ alkylene)-(C₄-C₂₀ cycloalkenyl), C_{zero}-C₄ alkylene)-((C₁₀-C₂₀)bi- or tricycloalkyl), C_{zero}-C₄ alkylene)-((C₁₀-C₂₀)bi- or tricycloalkenyl), —(C_{zero}-C₄ alkylene)-((3-20 membered)heterocycloalkyl), C_{zero}-C₄ alkylene)-(C₆-C₁₅ aryl) and —(C_{zero}-C₄ alkylene)-((5-15 membered)heteroaryl), wherein said heterocycloalkyl optionally contains from one to four ring double or triple bonds;
wherein R⁷ is optionally substituted with from one to six fluorine atoms or with from one to three substituents independently selected from the group R^{1a};
or two independently selected R⁷ groups may, together with the carbon atoms to which they are respectively attached, optionally form a five to fourteen membered cycloalkyl ring, a five to fourteen membered heterocycloalkyl ring, a ten to fourteen membered bicycloalkyl ring or a ten to fourteen membered bicycloheteroalkyl ring fused to the 6-membered heteroaryl ring containing X, Y, W and U of Formula I, wherein from one to three members of said heterocycloalkyl ring or said bicycloheteroalkyl ring are selected from N, O and S, and wherein said cycloalkyl, heterocycloalkyl, bicycloalkyl or bicylcoheteroalkyl ring optionally contains from one to three double bonds;
R⁸ is selected from —H and —C₁-C₆ alkyl;
or, when Z is —C(═NR⁸)CHR¹R², R⁸ and R¹ may together with the nitrogen and carbon atoms to which they are respectively attached optionally form a five to fourteen membered heteroaryl ring or a five to eight membered heterocycloalkyl ring, wherein said heteroaryl or heterocycloalkyl ring optionally contains from one to two further heteroatoms selected from N, O and S, and wherein said heterocycloalkyl ring optionally contains from one to three double bonds, and wherein said heteroaryl or heterocycloalkyl ring is optionally substituted with from one to three substituents independently selected from the group R^{1b};
or a pharmaceutically acceptable salt thereof.

2. The compound according to claim 1, wherein Z is —C(═O)CHR¹R² or —C(═O)C(═O)R^{1a} nd R² is —H, —OH or —OC(═O)CH₃.

3. The compound according to claim 2, wherein Z is —C(═O)CHR¹R² and R² is H, OH.

4. The compound according to claim 1, wherein Z is —R¹.

5. The compound according to claim 1, wherein R¹ is selected from —C₁-C₂₀ alkyl, —C₂-C₂₀ alkenyl, —C₂-C₂₀ alkynyl, —C₃-C₂₀ cycloalkyl, -(4-20 membered)heterocycloalkyl, —C₆-C₂₀ aryl and -(5-20 membered)heteroaryl.

6. The compound according to claim 4, wherein R¹ is selected from —C₁-C₂₀ alkyl, —C₂-C₂₀ alkenyl, —C₂-C₂₀ alkynyl, —C₃-C₂₀ cycloalkyl, -(4-20 membered)heterocycloalkyl, —C₆-C₂₀ aryl and -(5-20 membered)heteroaryl.

7. The compound according to claim 4 wherein R¹ is C₃-C₁₀ cycloalkyl, which optionally contains one or two double or triple bonds.

8. The compound according to claim 4 wherein R¹ is C₂-C₁₂ alkyl, C₃-C₁₁ cycloalkyl, C₅-C₈ cycloalkenyl, (3-11) membered, heterocycloalkyl, C₆-C₁₀ aryl, and 5-10 membered heteroaryl.

9. The compound according to claim 8 wherein R¹ is C₁-C₄ alkyl, substituted with from one to two substitutents, independently selected from the group consisiting of F, OH, O(C═O)Me, —(C₆-C₁₀)aryl or 5-10 membered heteroaryl.

10. The compound according to claim 8 wherein R¹ is C₃-C₈ monocyclalkyl, C₅-C₁₁ bi or tricycloalkyl, C₅-C₈ monocycloalkenyl, (C₇-C₁₁) bi or tricycloalkenyl, 3-8 membered heteromonocycloalkyl, (5-11 membered)heterobicycloalkyl, aryl or heteroaryl.

11. The compound according to claim 4 wherien R¹ is 1,2,3,4-tetrahydronaphthalen-2-yl, indan-2-yl, 2,6,7,8,9-tetrahydro-5H benzocycloketen-6-yl, 2-decahydro-naphthalen-2-yl or 2-[(2,3-dihydrobenzo-furan-6-yl methyl)].

12. The compound according to claim 1, wherein R¹ is selected from —C₁-C₁₀ alkyl, —C₂-C₁₀ alkenyl, —C₃-C₁₀ cycloalkyl, phenyl, thienyl and pyridyl, and wherein R¹ is optionally independently substituted with from one to two substituents independently selected from —C₁-C₄ alkyl, —C₁-C₄ alkoxy, —F, —Cl, —Br, —CF₃, phenyl and phenoxy.

13. The compound according to claim 12, wherein R¹ is selected from phenyl, thienyl, and pyridyl, and wherein R¹ is optionally independently substituted with from one to two substituents independently selected from —F, —Cl, —CH₃, —CF₃, phenyl and phenoxy.

14. The compound according to claim 13, wherein R¹ is phenyl which is optionally substituted with one or two substituents selected from F, Cl, CH₃, CF₃, phenyl or phenoxy.

15. The compound according to claim 1, wherein R³ is selected from —C₁-C₆ alkyl, allyl and —CH₂CH₂SCH₃ and the R³ is optionally substituted with one to six F.

16. The compound according to claim 15, wherein R³ is selected from Me, Et, n-Pr, i-Pr, n-Bu, i-Bu, s-Bu, t-Bu and —CH₂CH₂SCH₃.

17. The compound according to claim 1, wherein R⁵ is H.

18. The compound according to claim 1, wherein R⁷ is selected from —H, —C₁-C₁₂ alkyl, —C₂-C₁₂ alkenyl, —C₁-C₂₀ alkoxy, —F, —Cl, —Br, —I, —CN, —NO₂, —C₃-C₁₅ cycloalkyl, —C₁-C₆ alkylene-(C₆-C₁₀)aryl, —C₁-C₆ alkylene-(C₅-C₁₀)heteroaryl, —(C₁-C₆ alkylene)-(C₃-C₇)cycloalkyl, —(C₁-C₆ alkylene)-(C₅-C₇)heterocycloalkyl, -(3-15 membered)heterocycloalkyl, —C₆-C₁₅ aryl, -(5-15 membered)heteroaryl, —CHO, —C(═O)(C₁-C₁₅ alkyl), —C(═O)((5-15 membered)heterocycloalkyl), —C(═O)(C₅-C₁₅ aryl), —C(═O)((5-15 membered)heteroaryl), —C(═O)(C₅-C₁₅ cycloalkyl), —C(═O)O(C₁-C₈ alkyl), —C(═O)N(C₁-C₁₀ alkyl)(C₁-C₁₀ alkyl), —C(═O)N(C_{zero}-C₁₀ alkyl)(C₆-C₁₀ aryl), —C(═O)N(C_{zero}-C₁₀ alkyl)((5-10 membered)heteroaryl), —C(═O)N(C_{zero}-C₁₀ alkyl)((5-10 membered)heterocycloalkyl), —C(═O)N(C_{zero}-C₁₀ alkyl)(C₅-C₁₀ cycloalkyl), —SO₂—(C₁-C₆ alkyl), —SO₂—(C₃-C₈ cycloalkyl), —SO₂—(C₆-C₁₀ aryl), —SO₂-((5-10 membered)heteroaryl), —NR⁹R¹⁰, and —(C₁-C₁₁ alkyenel)-NR⁹R¹⁰ wherein said alkyl, cycloalkyl, heterocycloalkyl, aryl and heteroaryl are each optionally independently substituted with from one to three substituents independently selected from —F, —Cl, —Br, —I, —OH, —C₁-C₆ alkoxy, —C₂-C₆ alkenoxy, —C₂-C₆ alkynoxy, —NR⁹R¹⁰, —(C₁-C₁₁ alkyl)-NR⁹R¹⁰, —C(═O)R¹¹, —SO₂—R¹¹, —C(═O)OR², —C(═O)NR⁹R¹⁰, —SO₂—NR⁹R¹⁰—C₃-C₁₅ cycloalkyl, -(4-15 membered)heterocycloalkyl, —C₆-C₁₅ aryl, -(5-15 membered)heteroaryl, -(4-12 membered)heterocycloalkoxy, —C₆-C₁₂ aryloxy and -(6-12 membered)heteroaryloxy.

19. The compound according to claim 18, wherein R⁷ is selected from —C₁-C₁₂ alkyl, —C₂-C₁₂ alkenyl, —C₃-C₁₅ cycloalkyl, C₁-C₆ alkylene-(C₆-C₁₀)aryl, —C₁-C₆ alkylene-(C₅-C₁₀)heteroaryl, —(C₁-C₆ alkylene)-(C₃-C₇)cycloalkyl, —(C₁-C₆ alkylene)-(C₅-C₇)heterocycloalkyl, —NR⁹R¹⁰, and —(C₁-C₁₁ alkylene)-NR⁹R¹⁰ and 4-15 membered)heterocycloalkyl, and wherein said alkyl, alkenyl, cycloalkyl and heterocycloalkyl are each optionally independently substituted with from one to three substituents independently selected from —OH, —C₁-C₆ alkoxy, —C₂-C₆ alkenoxy, —C₂-C₆ alkynoxy, —C₆-C₁₅ aryl, -(5-15 membered)heteroaryl, —NR⁹R¹⁰ and —(C₁-C₇ alkyl)-NR⁹R¹⁰.

20. The compound according to claim 19, wherein R⁷ is selected from —C₁-C₁₂ alkyl, —C₂-C₁₂ alkenyl, —C₃-C₁₅ cycloalkyl, C₁-C₆ alkylene-(C₆-C₁₀)aryl, —C₁-C₆ alkylene-(C₅-C₁₀)heteroaryl, —(C₁-C₆ alkylene)-(C₃-C₇)cycloalkyl, —(C₁-C₆ alkylene)-(C₅-C₇)heterocycloalkyl, —NR⁹R¹⁰, and —(C₁-C₁₁ alkylene)-NR⁹R¹⁰ and -(4-15 membered)heterocycloalkyl, and wherein said alkyl, alkenyl, cycloalkyl and heterocycloalkyl are each optionally independently substituted with from one to three substituents independently selected from —OH, —C₁-C₆ alkoxy, —C₂-C₆ alkenoxy and —C₂-C₆ alkynoxy or NR⁹R¹⁰.

21. The compound according to claim 20, wherein R⁷ is selected from —C₁-C₁₂ alkyl, —C₂-C₁₂ alkenyl, C₁-C₆ alkylene-(C₆-C₁₀)aryl, —C₁-C₆ alkylene-(C₅-C₁₀)heteroaryl, —(C₁-C₆ alkylene)-(C₃-C₇)cycloalkyl, —(C₁-C₆ alkylene)-(C₅-C₇)heterocycloalkyl, and —C₃-C₁₅ cycloalkyl, and wherein
said alkyl, alkenyl, alkylene, and cycloalkyl are each optionally independently substituted with NR⁹R¹⁰.

22. The compound according to claim 1, wherein R⁷ is a -(4-15 membered) heterocycloalkyl, and wherein said heterocycloalkyl is optionally substituted with from one to three substituents independently selected from —OH, —C₁-C₆ alkyl, —C₂-C₆ alkenyl, —C₂-C₆ alkynyl, —C₁-C₆ alkoxy, —C₂-C₆ alkenoxy, —C₂-C₆ alkynoxy, —C₆-C₁₀ aryl and -(5-15 membered)heteroaryl.

23. The compound according to claim 1, wherein NR⁹R¹⁰ is selected from —N(C_{zero}-C₆ alkyl)(C_{zero}-C₁₋₂ alkyl), —N(C_{zero}-C₆ alkyl)(C₃-C₁₂ cycloalkyl), —N(C₃-C₆ cycloalkyl)(C₃-C₁₂ cycloalkyl) and N(C_{zero}-C₆ alkyl)((3-12 membered)heterocycloalkyl), and wherein said NR⁹R¹⁰ may optionally be substituted with from one to six fluorine atoms or with from one to three substituents independently selected from —OH, —NH₂, —NH(C₁-C₄ alkyl), —C₁-C₆ alkoxy, —C₂-C₆ alkenoxy and —C₂-C₆ alkynoxy, and wherein said NR⁹R¹⁰ may optionally contain one to three double or triple bonds.

24. The compound according to claim 1, wherein the heteroaryl containing X, Y, W, and U is pyridyl, pyrimidyl, pyrazinyl, pyridazinyl, quinolinyl, quinazolinyl, quinoxalinyl, cyclopentapyrimidine or dihydropyrrolopyrimidine.

25. The compound according to claim 1 having the formula:

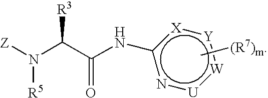

26. The compound according to claim 1 wherein Z is C(═O)CHR¹R², m is an integer from 0-3, R¹ is C₁-C₂₀ aryl, or which may be unsubstituted or substituted by 1 to 3 fluoro groups;
R² is hydrogen or OH;
R⁵ is hydrogen;
R³ is C₁-C₆ alkyl;
X, Y, U and W as CH or N, provided no more than 2 of X, Y, U and W are N;
m is 0, 1, 2 or 3;
R⁷ is H, alkyl, NR⁹R¹⁰, alkenyl, which alkyl, alkenyl groups are unsubstituted or substituted by 1-3 R^{1a} groups, R^{1a} is OH, C(═O)R¹¹, NR⁹R¹⁰, or C(═O)OR¹²;
R⁹ is hydrogen or C₁-C₆ alkyl and R¹⁰ is C₁-C₁₂ alkyl (C₀-C₄ alkylene), (C₆-C₁₀ aryl); (C₀-C₄alkylene) 5-15 membered heterocycloalkyl; R¹¹ is C₁-C₁₅ alkyl or H; R¹² is C₁-C₁₅alkyl;
wherein said alkyl or heterocycloalkyl group is optionally unsubstituted or substituted with one to six fluorine atoms or 1-3 substiuttents selected from alkyl, aryl, (C₀-C₄ alkylene), C₆-C₁₀ aryl, (C₀-C₄ alkylene) C₃-C₆ cycloalkyl, C(═O)OR¹², OH and said aryl group is unsubstituted or substituted with 1 to 3 substitutents independently selected from halo, OH, alkyl, hydroxyalkyl, aryloxy, alkoxy, CF₃;

27. The compound according to claim 26 wherein aryl is phenyl.

28. The compound according to claim 1 selected from the group consisting of:
2-[2-(3,5-Difluoro-phenyl)-acetylamino]-N-pyridin-2-yl-butyramide
N-(5-Bromo-pyridin-2-yl)-2-[2-(3,5-difluoro-phenyl)-acetylamino]-butyramide
2-[2-(3,5-Difluoro-phenyl)-acetylamino]-N-(5-iodo-pyridin-2-yl)-butyramide
2-[2-(3-phenoxy-phenyl)-acetylamino]-pentanoic acid pyrazin-2-ylamide
2-[2-(3-phenoxy-phenyl)-acetylamino]-pentanoic acid pyrimidin-2-ylamide
2-[2-(3-phenoxy-phenyl)-acetylamino]-pentanoic acid (6-chloro-pyridazin-3-yl)-amide
2-[2-(3-phenoxy-phenyl)-acetylamino]-pentanoic acid pyrimidin-4-ylamide
2-[2-(3-phenoxy-phenyl)-acetylamino]-pentanoic acid (4-methyl-pyrimidin-2-yl)-amide
2-[2-(5-Bromo-pyridin-3-yl)-acetylamino]-pentanoic acid pyrazin-2-ylamide
2-(2-Hydroxy-3-methyl-butyrylamino)-pentanoic acid pyrazin-2-ylamide
2-[2-(3-phenoxy-phenyl)-acetylamino]-pentanoic acid (4-chloro-6-methyl-pyrimidin-2-yl)-amide
2-[2-(3-phenoxy-phenyl)-acetylamino]-pentanoic acid (6-chloro-pyrazin-2-yl)-amide
2-[2-(3,5-Difluoro-phenyl)-acetylamino]-N-pyrazin-2-yl-butyramide
2-[2-(3,5-Difluoro-phenyl)-acetylamino]-pentanoic acid pyrazin-2-ylamide
2-(2-Hydroxy-3,3-dimethyl-butyrylamino)-pentanoic acid pyrazin-2-ylamide
2-[2-(3-phenoxy-phenyl)-acetylamino]-N-pyrazin-2-yl-propionamide
2-[2-(3,5-Difluoro-phenyl)-2-hydroxy-acetylamino]-pentanoic acid pyrazin-2-ylamide
2-[2-(3,5-Difluoro-phenyl)-2-hydroxy-acetylamino]-pentanoic acid pyrazin-2-ylamide
2-[2-(3-phenoxy-phenyl)-acetylamino]-pentanoic acid (6-dibutylamino-pyrazin-2-yl)-amide
2-[2-(3-phenoxy-phenyl)-acetylamino]-pentanoic acid [6-(1-ethyl-propylamino)-pyrazin-2-yl]-amide
2-[2-(3-phenoxy-phenyl)-acetylamino]-pentanoic acid (6-isopropylamino-pyrazin-2-yl)-amide
2-[2-(3-phenoxy-phenyl)-acetylamino]-pentanoic acid (6-ethylamino-pyrazin-2-yl)-amide
2-[2-(3-phenoxy-phenyl)-acetylamino]-pentanoic acid (6-butylamino-pyrazin-2-yl)-amide
2-[2-(3,5-Difluoro-phenyl)-acetylamino]-pentanoic acid (6-chloro-pyridazin-3-yl)-amide
2-[2-(3,5-Difluoro-phenyl)-acetylamino]-pentanoic acid pyrimidin-4-ylamide
2-[2-(3-phenoxy-phenyl)-acetylamino]-pentanoic acid [6-(butyl-methyl-amino)-pyrazin-2-yl]-amide
2-[2-(3,5-Difluoro-phenyl)-acetylamino]-pentanoic acid (5-trifluoromethyl-pyridin-2-yl)-amide
6-{2-[2-(3,5-Difluoro-phenyl)-acetylamino]-pentanoylamino}-nicotinamide
2-[2-(3,5-Difluoro-phenyl)-acetylamino]-pentanoic acid [5-(4-chloro-phenoxy)-pyrimidin-2-yl]-amide
2-[2-(3,5-Difluoro-phenyl)-acetylamino]-pentanoic acid (5-o-tolyloxy-pyrimidin-2-yl)-amide
2-[2-(3,5-Difluoro-phenyl)-acetylamino]-pentanoic acid (5-m-tolyloxy-pyrimidin-2-yl)-amide
2-[2-(3,5-Difluoro-phenyl)-acetylamino]-pentanoic acid [5-(3-tert-butyl-phenoxy)-pyrimidin-2-yl]-amide
2-[2-(3,5-Difluoro-phenyl)-acetylamino]-pentanoic acid (5-pentyloxy-pyrimidin-2-yl)-amide
2-[2-(3,5-Difluoro-phenyl)-acetylamino]-pentanoic acid [5-(3-trifluoromethyl-phenoxy)-pyrimidin-2-yl]-amide
2-[2-(3,5-Difluoro-phenyl)-acetylamino]-pentanoic acid [5-(naphthalen-2-yloxy)-pyrimidin-2-yl]-amide
2-[2-(3,5-Difluoro-phenyl)-acetylamino]-pentanoic acid [5-(4-methoxy-phenoxy)-pyrimidin-2-yl]-amide
2-[2-(3,5-Difluoro-phenyl)-acetylamino]-pentanoic acid (5-p-tolyloxy-pyrimidin-2-yl)-amide
2-[2-(3,5-Difluoro-phenyl)-acetylamino]-pentanoic acid (5-methyl-pyrimidin-2-yl)-amide
2-[2-(3,5-Difluoro-phenyl)-acetylamino]-pentanoic acid (5-bromo-pyrimidin-2-yl)-amide
2-[2-(3,5-Difluoro-phenyl)-acetylamino]-pentanoic acid (5-chloro-pyrimidin-2-yl)-amide
6-{2-[2-(3,5-Difluoro-phenyl)-acetylamino]-pentanoylamino}-nicotinic acid ethyl ester
3-(6-{2-[2-(3,5-Difluoro-phenyl)-acetylamino]-pentanoylamino}-pyridin-3-yl)-but-2-enoic acid methyl ester
2-[2-(3,5-Difluoro-phenyl)-acetylamino]-pentanoic acid (5-benzyl-pyridin-2-yl)-amide
4-(6-{2-[2-(3,5-Difluoro-phenyl)-acetylamino]-pentanoylamino}-pyridin-3-yl)-piperazine-1-carboxylic acid tert-butyl ester
2-[2-(3,5-Difluoro-phenyl)-acetylamino]-pentanoic acid (5-cyano-pyridin-2-yl)-amide
3-(6-{2-[2-(3,5-Difluoro-phenyl)-acetylamino]-pentanoylamino}-pyridin-3-yl)-butyric acid methyl ester
2-[2-(3,5-Difluoro-phenyl)-acetylamino]-pentanoic acid (6-chloro-pyridazin-3-yl)-amide
2-[2-(3,5-Difluoro-phenyl)-acetylamino]-pentanoic acid [5-(1-hydroxy-1-methyl-ethyl)-pyridin-2-yl]-amide
2-[2-(3,5-Difluoro-phenyl)-acetylamino]-pentanoic acid (5-bromo-pyrazin-2-yl)-amide
2-[2-(3,5-Difluoro-phenyl)-acetylamino]-pentanoic acid [5-(1-hydroxy-1-methyl-pentyl)-pyridin-2-yl]-amide
2-[2-(3,5-Difluoro-phenyl)-acetylamino]-pentanoic acid [5-(1-butyl-ylnyl)-pyridin-2-yl]-amide
2-[2-(3,5-Difluoro-phenyl)-acetylamino]-pentanoic acid {5-[methyl-(3-methyl-butyl)-amino]-pyrazin-2-yl}-amide
2-[2-(3,5-Difluoro-phenyl)-acetylamino]-pentanoic acid [5-(butyl-methyl-amino)-pyrazin-2-yl]-amide
2-[2-(3,5-Difluoro-phenyl)-acetylamino]-pentanoic acid [5-(butyl-ethyl-amino)-pyrazin-2-yl]-amide
2-[2-(3,5-Difluoro-phenyl)-acetylamino]-pentanoic acid [5-(cyclopropylmethyl-propyl-amino)-pyrazin-2-yl]-amide
2-[2-(3,5-Difluoro-phenyl)-acetylamino]-pentanoic acid [5-(hexyl-methyl-amino)-pyrazin-2-yl]-amide
2-[2-(3,5-Difluoro-phenyl)-acetylamino]-pentanoic acid (6-[methyl-(3-methyl-butyl)-amino]-pyridazin-3-yl}-amide
2-[2-(3,5-Difluoro-phenyl)-acetylamino]-pentanoic acid [6-(butyl-methyl-amino)-pyridazin-3-yl]-amide
2-[2-(3,5-Difluoro-phenyl)-acetylamino]-pentanoic acid [6-(1-ethyl-propylamino)-pyridazin-3-yl]-amide
2-[2-(3,5-Difluoro-phenyl)-acetylamino]-pentanoic acid [6-(butyl-ethyl-amino)-pyridazin-3-yl]-amide
2-[2-(3,5-Difluoro-phenyl)-acetylamino]-pentanoic acid [6-(hexyl-methyl-amino)-pyridazin-3-yl]-amide
3-(2-{2-[2-(3,5-Difluoro-phenyl)-acetylamino]-pentanoylamino}-pyrimidin-5-yl)-but-2-enoic acid methyl ester
2-[2-(3,5-Difluoro-phenyl)-acetylamino]-pentanoic acid (5-bromo-pyridin-2-yl)-amide
2-[2-(3,5-Difluoro-phenyl)-acetylamino]-pentanoic acid (5-morpholin-4-yl-pyrazin-2-yl)-amide
2-[2-(3,5-Difluoro-phenyl)-acetylamino]-pentanoic acid (6-methyl-pyridin-2-yl)-amide
2-[2-(3,5-Difluoro-phenyl)-acetylamino]-pentanoic acid (5-phenethylamino-pyrazin-2-yl)-amide
2-[2-(3,5-Difluoro-phenyl)-acetylamino]-pentanoic acid [5-(benzyl-methyl-amino)-pyrazin-2-yl]-amide
2-[2-(3,5-Difluoro-phenyl)-acetylamino]-pentanoic acid (5-dibenzylamino-pyrazin-2-yl)-amide
2-[2-(3,5-Difluoro-phenyl)-acetylamino]-pentanoic acid [5-(1-methoxymethyl-propylamino)-pyrazin-2-yl]-amide
2-[2-(3,5-Difluoro-phenyl)-acetylamino]-pentanoic acid (5-isopropylamino-pyrazin-2-yl)-amide
2-[2-(3,5-Difluoro-phenyl)-acetylamino]-pentanoic acid [5-(2-hydroxy-ethylamino)-pyrazin-2-yl]-amide
2-[2-(3,5-Difluoro-phenyl)-acetylamino]-pentanoic acid (5-benzylamino-pyrazin-2-yl)-amide
2-[2-(3,5-Difluoro-phenyl)-acetylamino]-pentanoic acid [5-(benzyl-ethyl-amino)-pyrazin-2-yl]-amide
2-[2-(3,5-Difluoro-phenyl)-acetylamino]-pentanoic acid (5-pyrrolidin-1-yl-pyrazin-2-yl)-amide
2-[2-(3-Fluoro-5-pyrrolidin-1-yl-phenyl)-acetylamino]-pentanoic acid (5-pyrrolidin-1-yl-pyrazin-2-yl)-amide
2-[2-(3,5-Difluoro-phenyl)-acetylamino]-pentanoic acid pyrazin-2-ylamide
3-(6-{2-[2-(3,5-Difluoro-phenyl)-acetylamino]-pentanoylamino}-pyridin-3-yl)-acryl ic acid methyl ester
2-[2-(3,5-Difluoro-phenyl)-acetylamino]-pentanoic acid [5-(1-methyl-3-oxo-but-1-enyl)-pyridin-2-yl]-amide
2-[2-(3,5-Difluoro-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid [5-(pyrazin-2-ylamino)-pyrazin-2-yl]-amide
2-[2-(3,5-Difluoro-phenyl)-acetylamino]-pentanoic acid [5-(1-hydroxy-1-methyl-ethyl)-pyrazin-2-yl]-amide
2-[2-(3,5-Difluoro-phenyl)-acetylamino]-pentanoic acid [5-(3-amino-1-methyl-butyl)-pyridin-2-yl]-amide
2-[2-(3,5-Difluoro-phenyl)-acetylamino]-pentanoic acid [5-(1-methyl-3-methylamino-butyl)-pyridin-2-yl]-amide
2-[2-(3,5-Difluoro-phenyl)-acetylamino]-pentanoic acid {5-[3-(2-hydroxy-ethylamino)-1-methyl-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid [5-(3-hydroxy-1-methyl-butyl)-pyridin-2-yl]-amide
2-[2-(3,5-Difluoro-phenyl)-acetylamino]-pentanoic acid {5-[3-(2,2,2-trifluoro-ethylamino)-butyl]-pyridin-2-yl}-amide
3-(5-{2-[2-(3,5-Difluoro-phenyl)-acetylamino]-pentanoylamino}-pyrazin-2-ylamino)-propionic acid methyl ester
2-[2-(3,5-Difluoro-phenyl)-acetylamino]-pentanoic acid (5-acetyl-pyridin-2-yl)-amide
2-[2-(3,5-Difluoro-phenyl)-acetylamino]-pentanoic acid [5-(3-oxo-but-1-enyl)-pyridin-2-yl]-amide
3-(6-{2-[2-(3,5-Difluoro-phenyl)-acetylamino]-pentanoylamino}-pyridin-3-yl)-propionic acid methyl ester
6-{2-[2-(3,5-Difluoro-phenyl)-acetylamino]-pentanoylamino}-nicotinic acid methyl ester
2-[2-(3,5-Difluoro-phenyl)-acetylamino]-pentanoic acid [5-(3-oxo-butyl)-pyridin-2-yl]-amide
2-[2-(3,5-Difluoro-phenyl)-acetylamino]-pentanoic acid [5-(1-hydroxy-ethyl)-pyridin-2-yl]-amide
2-[2-(3,5-Difluoro-phenyl)-acetylamino]-pentanoic acid [5-(3-methylamino-butyl)-pyridin-2-yl]-amide
2-[2-(3,5-Difluoro-phenyl)-acetylamino]-pentanoic acid [5-(3-ethylamino-butyl)-pyridin-2-yl]-amide
2-[2-(3,5-Difluoro-phenyl)-acetylamino]-pentanoic acid [5-(3-propylamino-butyl)-pyridin-2-yl]-amide
2-[2-(3,5-Difluoro-phenyl)-acetylamino]-pentanoic acid [5-(3-isopropylamino-butyl)-pyridin-2-yl]-amide
2-[2-(3,5-Difluoro-phenyl)-acetylamino]-pentanoic acid [5-(3-butylamino-butyl)-pyridin-2-yl]-amide
2-[2-(3,5-Difluoro-phenyl)-acetylamino]-pentanoic acid [5-(3-isobutylamino-butyl)-pyridin-2-yl]-amide
2-[2-(3,5-Difluoro-phenyl)-acetylamino]-pentanoic acid {5-[3-(3-methyl-butylamino)-butyl]-pyridin-2-yl}-amide
2-[2-(3,5-Difluoro-phenyl)-acetylamino]-pentanoic acid {5-[1-(3-methyl-butylamino)-ethyl]-pyridin-2-yl}-amide
6-{2-[2-(3,5-Difluoro-phenyl)-acetylamino]-pentanoylamino}-nicotinic acid
2-[2-(3,5-Difluoro-phenyl)-acetylamino]-pentanoic acid [5-(1-isobutylamino-ethyl)-pyridin-2-yl]-amide
2-[2-(3,5-Difluoro-phenyl)-acetylamino]-pentanoic acid {5-[1-(3,3-dimethyl-butylamino)-ethyl]-pyridin-2-yl}-amide
2-[2-(3,5-Difluoro-phenyl)-acetylamino]-pentanoic acid {5-[1-(1-ethyl-propylamino)-ethyl]-pyridin-2-yl}-amide
2-[2-(3,5-Difluoro-phenyl)-acetylamino]-pentanoic acid {5-[1-(2,2,2-trifluoro-ethylamino)-ethyl]-pyridin-2-yl}-amide
2-[2-(3,5-Difluoro-phenyl)-acetylamino]-pentanoic acid [5-(1-butylamino-ethyl)-pyridin-2-yl]-amide
2-[2-(3,5-Difluoro-phenyl)-acetylamino]-pentanoic acid [5-(1-morpholin-4-yl-ethyl)-pyridin-2-yl]-amide
2-[2-(3,5-Difluoro-phenyl)-acetylamino]-pentanoic acid [5-(1-benzylamino-ethyl)-pyridin-2-yl]-amide
2-[2-(3,5-Difluoro-phenyl)-acetylamino]-pentanoic acid [5-(1-cyclopropylamino-ethyl)-pyridin-2-yl]-amide
2-[2-(3,5-Difluoro-phenyl)-acetylamino]-pentanoic acid [5-(1-cyclopropylamino-ethyl)-pyridin-2-yl]-amide
2-[2-(3,5-Difluoro-phenyl)-acetylamino]-pentanoic acid {5-[1-(1-benzyl-pyrrolidin-3-ylamino)-ethyl]-pyridin-2-yl}-amide
2-[2-(3,5-Difluoro-phenyl)-acetylamino]-pentanoic acid {5-[1-(1-benzyl-pyrrolidin-3-ylamino)-ethyl]-pyridin-2-yl}-amide
2-[2-(3,5-Difluoro-phenyl)-acetylamino]-pentanoic acid [5-(1-cyclobutylamino-ethyl)-pyridin-2-yl]-amide
2-[2-(3,5-Difluoro-phenyl)-acetylamino]-pentanoic acid [5-(3-oxo-butyl)-pyrazin-2-yl]-amide
2-[2-(3,5-Difluoro-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid [5-(3-morpholin-4-yl-butyl)-pyridin-2-yl]-amide
2-[2-(3,5-Difluoro-phenyl)-acetylamino]-pentanoic acid [5-(3-pyrrolidin-1-yl-butyl)-pyridin-2-yl]-amide
2-[2-(3,5-Difluoro-phenyl)-acetylamino]-pentanoic acid [5-(3-benzylamino-butyl)-pyridin-2-yl]-amide
2-[2-(3,5-Difluoro-phenyl)-acetylamino]-pentanoic acid [5-(3-cyclopropylamino-butyl)-pyridin-2-yl]-amide
2-[2-(3,5-Difluoro-phenyl)-acetylamino]-pentanoic acid [5-(3-cyclobutylamino-butyl)-pyridin-2-yl]-amide
2-[2-(3,5-Difluoro-phenyl)-acetylamino]-pentanoic acid [5-(1-pyrrolidin-1-yl-ethyl)-pyridin-2-yl]-amide
2-[2-(3,5-Difluoro-phenyl)-acetylamino]-pentanoic acid {5-[1-(4-methyl-piperazin-1-yl)-ethyl]-pyridin-2-yl}-amide
2-[2-(3,5-Difluoro-phenyl)-acetylamino]-pentanoic acid {5-[1-(4-methyl-piperazin-1-yl)-ethyl]-pyridin-2-yl}-amide
2-[2-(3,5-Difluoro-phenyl)-acetylamino]-pentanoic acid {5-[1-(2-hydroxy-ethylamino)-ethyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid {5-[1-methyl-3-(2,2,2-trifluoro-ethylamino)-butyl]-pyridin-2-yl}-amide
2-[2-(3,5-Difluoro-phenyl)-acetylamino]-pentanoic acid [5-(3-cyclopropylamino-butyl)-pyridin-2-yl]-amide
2-[2-(3,5-Difluoro-phenyl)-acetylamino]-pentanoic acid [5-(3-cyclopropylamino-butyl)-pyridin-2-yl]-amide
2-[2-(3,5-Difluoro-phenyl)-acetylamino]-pentanoic acid [5-(3-oxo-propenyl)-pyridin-2-yl]-amide
2-[2-(3,5-Difluoro-phenyl)-acetylamino]-pentanoic acid [5-(3-hydroxy-3-methyl-butyl)-pyridin-2-yl]-amide
2-[2-(3,5-Difluoro-phenyl)-acetylamino]-pentanoic acid (5-hydroxymethyl-pyridin-2-yl)-amide
2-[2-(3,5-Difluoro-phenyl)-acetylamino]-pentanoic acid [5-(3-cyclobutylamino-butyl)-pyridin-2-yl]-amide
2-[2-(3,5-Difluoro-phenyl)-acetylamino]-pentanoic acid [5-(3-cyclobutylamino-butyl)-pyridin-2-yl]-amide
2-[2-(3,5-Difluoro-phenyl)-acetylamino]-pentanoic acid [5-(3-benzylamino-1-methyl-butyl)-pyridin-2-yl]-amide
2-[2-(3,5-Difluoro-phenyl)-acetylamino]-pentanoic acid [5-(3-benzylamino-1-methyl-butyl)-pyridin-2-yl]-amide
2-[2-(3,5-Difluoro-phenyl)-acetylamino]-pentanoic acid {5-[3-(1-phenyl-ethylamino)-butyl]-pyridin-2-yl}-amide
2-[2-(3,5-Difluoro-phenyl)-acetylamino]-pentanoic acid (5-[3-(1-phenyl-ethylamino)-butyl]-pyridin-2-yl}-amide
2-[2-(3,5-Difluoro-phenyl)-acetylamino]-pentanoic acid {5-[3-(2-hydroxy-1-phenyl-ethylamino)-butyl]-pyridin-2-yl}-amide
2-[2-(3,5-Difluoro-phenyl)-acetylamino]-pentanoic acid {5-[3-(2-hydroxy-1-phenyl-ethylamino)-butyl]-pyridin-2-yl}-amide
2-[2-(3,5-Difluoro-phenyl)-acetylamino]-pentanoic acid {5-[3-(2-trifluoromethyl-benzylamino)-butyl]-pyridin-2-yl}-amide
2-[2-(3,5-Difluoro-phenyl)-acetylamino]-pentanoic acid {5-[3-(3-trifluoromethyl-benzylamino)-butyl]-pyridin-2-yl}-amide
2-[2-(3,5-Difluoro-phenyl)-acetylamino]-pentanoic acid {5-[3-(2,4-difluoro-benzylamino)-butyl]-pyridin-2-yl}-amide
2-[2-(3,5-Difluoro-phenyl)-acetylamino]-pentanoic acid {5-[3-(4-chloro-benzylamino)-butyl]-pyridin-2-yl}-amide
2-[2-(3,5-Difluoro-phenyl)-acetylamino]-pentanoic acid {5-[3-(4-methoxy-benzylamino)-butyl]-pyridin-2-yl}-amide
2-[2-(3,5-Difluoro-phenyl)-acetylamino]-pentanoic acid {5-[3-(2-fluoro-benzylamino)-butyl]-pyridin-2-yl}-amide
2-[2-(3,5-Difluoro-phenyl)-acetylamino]-pentanoic acid {5-[3-(3-chloro-benzylamino)-butyl]-pyridin-2-yl}-amide
2-[2-(3,5-Difluoro-phenyl)-acetylamino]-pentanoic acid {5-[3-(4-fluoro-benzylamino)-butyl]-pyridin-2-yl}-amide
2-[2-(3,5-Difluoro-phenyl)-acetylamino]-pentanoic acid {5-[3-(3-fluoro-benzylamino)-butyl]-pyridin-2-yl}-amide
2-[2-(3,5-Difluoro-phenyl)-acetylamino]-pentanoic acid {5-[3-(4-trifluoromethyl-benzylamino)-butyl]-pyridin-2-yl}-amide
2-[2-(3,5-Difluoro-phenyl)-acetylamino]-pentanoic acid [5-(3-benzylamino-butyl)-pyridin-2-yl]-amide
2-[2-(3,5-Difluoro-phenyl)-acetylamino]-pentanoic acid [5-(3-benzylamino-butyl)-pyridin-2-yl]-amide
2-[2-(3,5-Difluoro-phenyl)-acetylamino]-pentanoic acid (5-formyl-pyridin-2-yl)-amide
2-[2-(3,5-Difluoro-phenyl)-acetylamino]-pentanoic acid {5-[1-(4-methyl-piperidin-1-yl)-ethyl]-pyridin-2-yl}-amide
2-[2-(3,5-Difluoro-phenyl)-acetylamino]-pentanoic acid (5-ylnyl-pyrazin-2-yl)-amide
2-[2-(3,5-Difluoro-phenyl)-acetylamino]-pentanoic acid {5-[1-(4-phenyl-piperidin-1-yl)-ethyl]-pyridin-2-yl}-amide
2-[2-(3,5-Difluoro-phenyl)-acetylamino]-pentanoic acid (5-formyl-pyrazin-2-yl)-amide
2-[2-(3-Trifluoromethyl-phenyl)-acetylamino]-pentanoic acid [5-(3-oxo-butyl)-pyridin-2-yl]-amide
2-[2-(3-Trifluoromethyl-phenyl)-acetylamino]-pentanoic acid [5-(1-methyl-3-oxo-butyl)-pyridin-2-yl]-amide
2-[2-(3-Trifluoromethoxy-phenyl)-acetylamino]-pentanoic acid [5-(1-methyl-3-oxo-butyl)-pyridin-2-yl]-amide
2-[2-(3,5-Difluoro-phenyl)-acetylamino]-pentanoic acid (5-butylaminomethyl-pyrazin-2-yl)-amide
2-[2-(3,5-Difluoro-phenyl)-acetylamino]-pentanoic acid {5-[(3,3-dimethyl-butylamino)-methyl]-pyrazin-2-yl}-amide
2-[2-(3,5-Difluoro-phenyl)-acetylamino]-pentanoic acid {5-[(1-phenyl-propylamino)-methyl]-pyrazin-2-yl}-amide
2-[2-(3,5-Difluoro-phenyl)-acetylamino]-pentanoic acid {5-[(1-benzyl-pyrrolidin-3-ylamino)-methyl]-pyrazin-2-yl}-amide
2-[2-(3,5-Difluoro-phenyl)-acetylamino]-pentanoic acid [5-(1-hydroxy-ethyl)-pyrazin-2-yl]-amide
2-[2-(3,5-Difluoro-phenyl)-acetylamino]-pentanoic acid [5-(1-hydroxy-pentyl)-pyrazin-2-yl]-amide
2-[2-(3,5-Difluoro-phenyl)-acetylamino]-pentanoic acid [5-(4-methyl-piperazin-1-yl methyl)-pyrazin-2-yl]-amide
2-[2-(3,5-Difluoro-phenyl)-acetylamino]-pentanoic acid (5-hydroxymethyl-pyrazin-2-yl)-amide
2-[2-(3,5-Difluoro-phenyl)-acetylamino]-pentanoic acid [5-phenethylamino-methyl)-pyrazin-2-yl]-amide
2-[2-(3,5-Difluoro-phenyl)-acetylamino]-pentanoic acid [5-(isobutylamino-methyl)-pyrazin-2-yl]-amide
2-[2-(3,5-Difluoro-phenyl)-acetylamino]-pentanoic acid [5-(benzylamino-methyl)-pyrazin-2-yl]: amide
2-[2-(3,5-Difluoro-phenyl)-acetylamino]-pentanoic acid {5-[(3-methyl-butylamino)-methyl]-pyrazin-2-yl}-amide
2-[2-(3,5-Difluoro-phenyl)-acetylamino]-pentanoic acid {5-[(4-chloro-phenyl)-hydroxy-methyl]-pyrazin-2-yl}-amide
6-[2-(6,8-difluoro-1,2,3,4-tetrahydro-naphthalen-2-ylamino)-pentanoylamino]-nicotinic acid methyl ester
2-[2-(3,5-difluoro-phenyl)-acetylamino]-pentanoic acid [5-(1-hydroxy-1-methyl-allyl)-pyridin-2-yl]-amide
2-[2-(3,5-difluoro-phenyl)-acetylamino]-pentanoic acid {5-[1-(4-methyl-piperazin-1-yl)-ethyl]-pyridin-2-yl}-amide
2-[2-(3,5-difluoro-phenyl)-acetylamino]-pentanoic acid (5-{[methyl-(3-methyl-butyl)-amino]-methyl}-pyrazin-2-yl)-amide
2-[2-(3,5-Difluoro-phenyl)-acetylamino]-pentanoic acid {5-[(3-hydroxy-butylamino)-methyl]-pyrazin-2-yl}-amide
2-[2-(3,5-difluoro-phenyl)-acetylamino]-pentanoic acid {5-[(1-phenyl-ethylamino)-methyl]-pyrazin-2-yl}-amide
2-[2-(3,5-difluoro-phenyl)-acetylamino]-pentanoic acid (5-acetyl-pyrazin-2-yl)-amide
6-[2-(6,8-Difluoro-1,2,3,4-tetrahydro-naphthalen-2-ylamino)-pentanoylamino]-nicotinic acid methyl ester
2-(6,8-Difluoro-1,2,3,4-tetrahydro-naphthalen-2-ylamino)-pentanoic acid (5-bromo-pyrazin-2-yl)-amide
2-(6,8-Difluoro-1,2,3,4-tetrahydro-naphthalen-2-ylamino)-pentanoic acid pyrazin-2-ylamide
2-(6,8-Difluoro-1,2,3,4-tetrahydro-naphthalen-2-ylamino)-pentanoic acid (5-cyano-pyrazin-2-yl)-amide
2-(6,8-Difluoro-1,2,3,4-tetrahydro-naphthalen-2-ylamino)-pentanoic acid [5-(3-oxo-butyl)-pyrazin-2-yl]-amide
2-(5,7-Difluoro-1,2,3,4-tetrahydro-naphthalen-2-ylamino)-pentanoic acid {5-[3-(1-phenyl-ethylamino)-butyl]-pyridin-2-yl}-amide
or pharmaceutically acceptable salts thereof.

29. A pharmaceutical composition for inhibiting Aβ-peptide production in a mammal, comprising an amount of the compound according to claim 1 or a pharmaceutically acceptable salt thereof that is effective in inhibiting Aβ-production, and a pharmaceutically acceptable carrier therefor.

30. A pharmaceutical composition for treating a disease or a condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome in a mammal, comprising an amount of the compound according to claim 1 or a pharmaceutically acceptable salt thereof that is effective in treating such disease or condition, and a pharmaceutically acceptable carrier therefor.

31. A method of inhibiting Aβ-peptide production in a mammal, comprising administering to said mammal an amount of the compound according to claim 1 or a pharmaceutically acceptable salt thereof that is effective in inhibiting Ap-production.

32. A method of treating a disease or condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome in a mammal, comprising administering to said mammal an amount of the compound according to claim 1 or a pharmaceutically acceptable salt thereof that is effective in treating such condition.

33. A pharmaceutical composition for treating a disease or condition associated with Aβ-peptide production in a mammal, comprising (a) the compound according to claim 1, or a pharmaceutically acceptable salt thereof; (b) an active agent selected from the group consisting of a memory enhancement agent, antidepressant, anxiolytic, antipsychotic agent, sleep disorder agent, anti-inflammatory agent, anti-oxidant agent, cholesterol modulating agent and anti-hypertensive agent; and (c) a pharmaceutically acceptable carrier; wherein (a) and (b) are present in amounts that render the composition effective in treating such disease or condition.

34. A pharmaceutical composition for treating a disease or condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome, in a mammal, comprising (a) the compound according to claim 1, or a pharmaceutically acceptable salt thereof; (b) an active agent selected from the group consisting of a memory enhancement agent, antidepressant, anxiolytic, antipsychotic agent, sleep disorder agent, anti-inflammatory agent, anti-oxidant agent, cholesterol modulating agent and anti-hypertensive agent; and (c) a pharmaceutically acceptable carrier; wherein the (a) and (b) are present in amounts that render the composition effective in treating such disease or condition.

35. A method of treating a disease or condition associated with Aβ-peptide production in a mammal, comprising administering to said mammal (a) the compound according to claim 1, or a pharmaceutically acceptable salt thereof; and (b) an active agent selected from the group consisting of a memory enhancement agent, antidepressant, anxiolytic, antipsychotic agent, sleep disorder agent, anti-inflammatory agent, anti-oxidant agent, cholesterol modulating agent and anti-hypertensive agent; wherein (a) and (b) are present in amounts that render the composition effective in treating such disease or condition.

36. A method of treating a disease or condition selected from the group consisting of Alzheimer's disease, hereditary cerebral hemorrhage with amyloidosis, cerebral amyloid angiopathy, a prion-mediated disease, inclusion body myositis, stroke, multiple sclerosis and Down's Syndrome, in a mammal, comprising administering to said mammal (a) the compound according to claim 1, or a pharmaceutically acceptable salt thereof; and (b) an active agent selected from the group consisting of a memory enhancement agent, antidepressant, anxiolytic, antipsychotic agent, sleep disorder agent, anti-inflammatory agent, anti-oxidant agent, cholesterol modulating agent and anti-hypertensive agent; wherein (a) and (b) are present in amounts that render the composition effective in treating such disease or condition.

37. A method of treating Alzheimer's disease in a mammal, comprising administering to said mammal (a) the compound according to claim 1, or a pharmaceutically acceptable salt thereof; and (b) an active agent selected from the group consisting of a memory enhancement agent, antidepressant, anxiolytic, antipsychotic agent, sleep disorder agent, anti-inflammatory agent, anti-oxidant agent, cholesterol modulating agent and anti-hypertensive agent; wherein (a) and (b) are present in amounts that render the composition effective in treating such disease or condition.
